(19) United States
(12) Patent Application Publication
(10) Pub. No.: US 2002/0133296 A1
(43) Pub. Date: Sep. 19, 2002

(54) Classification of polypeptides by ligand geometry and related methods

(76) Inventors: Daniel S. Sem, San Diego, CA (US); Mark Hansen, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/747,174

(22) Filed: Dec. 22, 2000

(51) Int. Cl. G06F 19/00; G01N 33/48; G01N 33/50

(52) U.S. Cl. 702/19; 530/388.1

(57) ABSTRACT

The invention provides a method for identifying a pharmacocluster. The method includes the steps of (a) determining bound conformations of a ligand bound to different polypeptides, and (b) clustering two or more bound conformations of the ligand having substantially the same bound conformation, thereby identifying a pharmacocluster. The invention also provides a method for identifying a member of a pharmacocluster. The invention also provides a method for identifying a polypeptide pharmacofamily. The method includes the steps of (a) determining bound conformations of a ligand bound to different polypeptides of a polypeptide family, and (b) identifying two or more bound conformations of the ligand having substantially different bound conformations, thereby identifying at least two polypeptide pharmacofamilies exhibiting binding specificity for the two or more substantially different bound conformations of the ligand.

### BACKGROUND OF THE INVENTION

The present invention relates generally to interactions between ligands and polypeptides and more specifically to determining structure-related properties of a ligand when bound to different polypeptides.

Structure determination plays a central role in chemistry and biology due to the correlation between the structure of a molecule and its function. Although a full understanding of this correlation is not yet established, one can gain insight into the function of a molecule from its deduced structure. Thus, the structure can provide a strong basis for formulating experiments to determine function. Conversely, the eventual disclosure of a structure for a well studied molecule can have a significant effect in converging apparently disparate observations of function into a consistent description of the molecule's activity.

Practical applications which are becoming increasingly dependent upon structure information include, for example, the production of therapeutic drugs. Therapeutic drugs can be designed by synthesizing a molecule that mimics a ligand known to interact with a target receptor. Alternatively, a therapeutic drug can be designed by computer assisted methods in which a molecule is designed to dock to a binding site on a receptor of known structure. By structure-based methods such as these, lead compounds can be identified for further development.

Using a similar structure based approach a receptor can be engineered to yield improved or novel functions. For example, changes can be made at a ligand binding site in a polypeptide receptor based on the known structure of the receptor. Given that a polypeptide receptor can contain hundreds or even thousands of amino acid residues, of which only a few may contact a ligand, structural information is useful in identifying where changes should be made in the polypeptide to alter ligand binding. Polypeptide receptors engineered as such can be used for a variety of practical applications including, for example, industrial catalysis, therapeutics, and bioremediation.

Although methods for structure determination are evolving, it is currently difficult, costly and time consuming to determine the structure of a polypeptide or ligand. It can often be even more difficult to produce a polypeptide-ligand complex in a condition allowing determination of a structure for the bound complex. Resorting to determining a structure for the receptor individually can have limited value, particularly if the location of ligand binding is difficult to identify due to the large size of most polypeptide receptors. Similarly, determination of a structure of an unbound ligand can have limited usefulness because an unbound ligand has multiple conformations and the most stable conformation of an unbound ligand is often different from its conformation when bound to a receptor.

Theoretical modeling of ligand-polypeptide interactions is one alternative that has been attempted in cases where the structure of the polypeptide-ligand complex is not available. In this approach a ligand is fitted to a structure of a polypeptide. The polypeptide structure used can be determined empirically or theoretically. Theoretical determination of a hypothetical molecular structure for a polypeptide by ab initio methods is a relatively undeveloped method. Another theoretical approach, referred to as homology modeling, has been used to infer structure based on comparison with molecules of known structure.

The successful application of homology modeling to determining polypeptide-ligand interactions relies upon choosing a correct polypeptide template for comparison. In most cases criteria for comparison are unavailable or unreliable. For example, it is common to produce a hypothetical structure of a target polypeptide based on the empirically determined structure of a template polypeptide having similar sequence. However, similarities in sequence do not always yield similar structures and conversely, similar structures have been observed for two polypeptides having significantly diverged sequences.

Thus, there exists a need for efficient methods to identify properties of a ligand that confer binding specificity for polypeptide receptors. A need also exists for methods to classify polypeptides and ligands according to structural characteristics. The present invention satisfies this need and provides related advantages as well.

### SUMMARY OF THE INVENTION

The invention provides a method for identifying a pharmacocluster. The method includes the steps of (a) determining bound conformations of a ligand bound to different polypeptides, and (b) clustering two or more bound conformations of the ligand having substantially the same bound conformation, thereby identifying a pharmacocluster. The invention also provides a method for identifying a member of a pharmacocluster. The invention also provides a method for identifying a polypeptide pharmacofamily. The method includes the steps of (a) determining bound conformations of a ligand bound to different polypeptides of a polypeptide family, and (b) identifying two or more bound conformations of the ligand having substantially different bound conformations, thereby identifying at least two polypeptide pharmacofamilies exhibiting binding specificity for the two or more substantially different bound conformations of the ligand.

### BRIEF DESCRIPTION OF THE DRAWINGS

FIG. 1 shows pharmacoclusters identified from a database of 156 bound structures of nicotinamide adenine dinucleotide or nicotinamide adenine dinucleotide phosphate. Structures were generated using the overlay function in INSIGHT98 (Molecular Simulations Inc., San Diego, Calif.).

FIG. 2 shows the nomenclature used herein for atom names in the NAD(P) molecule.

FIG. 3 shows conformer models with interacting atoms from bound polypeptide and ordered waters overlayed. Models in parts A through H were derived from pharmacoclusters 1-8, respectively as described in the Examples. Overlayed atoms and waters are identified as either hydrogen bond donors (donors), hydrogen bond acceptors (acceptors), sulfurs (sulfurs), waters (waters), or atoms that can be hydrogen bond acceptors or hydrogen bond donors (acceptors/donors) according to the legends under each conformer model.

FIG. 4 shows a portion of a 2D [¹H,¹H] NOESY spectrum recorded with a 0.2 ml sample of 1 mM NADP and 200 μM of enzyme 1-deoxy D-xylulose 5-phosphate reductoisomerase (DOXP). Atoms are identified according to FIG. 2. Spectra are reported as parts per million (ppm). Since the ligand is in fast exchange and is in excess over polypeptide, cross peaks represent transferred NOEs.

FIG. 5 shows high affinity binding of compound TTE0001.001.A07 to polypeptide enzymes of pharmacofamily 1 (panel A) and pharmacofamily 8 (panel B). Double reciprocal plots of reaction rate versus concentration of NADH (panel A) or NADPH (panel B) are shown for each enzyme in the presence of various concentrations of compound TTE0001.001.A07. Concentrations of compound TTE0001.001.A07 shown to the right of the plot A correspond 7.1 μM (open triangles), 3.6 μM (closed triangles), 1.8 μM (open circles) and no added compound (closed circles). Concentrations of compound TTE0001.001.A07 shown to the right of the plot B correspond 56.2 μM (open triangles), 37.5 μM (closed triangles), 18.7 μM (open circles) and no added compound (closed circles). Inhibitory dissociation constants (Kᵢₛ) determined from the data are shown in the upper left corner of the respective plot.

FIG. 6 shows high affinity binding of compound TTE0001.002.D02 to a polypeptide enzyme of pharmacofamily 1. A double reciprocal plot of reaction rate versus concentration of NADH is shown for the enzyme in the presence of various concentrations of compound TTE0001.002.D02. Concentrations of compound TTE0001.002.D02 shown to the right of the plot A correspond 20.6 μM (open triangles), 13.7 μM (closed triangles), 6.9 μM (open circles) and no added compound (closed circles). An inhibitory dissociation constant (Kᵢₛ) determined from the data is shown in the upper left corner of the plot.

FIG. 7 shows a pharmacophore model derived from the coordinates presented in Table 3 for pharmacofamily 1. FIG. 7A shows a feature of the pharmacophore model including a volume defining the shape of conformer model 1 which is indicated by grey spheres and superimposed on the conformer model having coordinates listed in Table 3C. FIG. 7B shows three features of the pharmacophore model including a hydrophobic region of the nicotinamide ring, a hydrogen bond acceptor positioned at the averaged coordinates for the location of 17 hydrogen bond acceptors in the polypeptides of pharmacofamily 1, and a hydrogen bond donor positioned where a hydrogen bond donor of a ligand would be expected to have favorable interactions with hydrogen bond acceptors observed in 11 of the 17 polypeptides in pharmacofamily 1. FIG. 7C shows a combination of features of FIGS. 7A and 7B present in a pharmacophore model and superimposed on the conformer model.

### DETAILED DESCRIPTION OF THE INVENTION

The invention provides pharmacoclusters and methods for identifying a pharmacocluster from bound conformations of a ligand bound to different polypeptides. The methods are applicable for identifying a conformation-dependent property of a ligand based on bound conformations of the ligand in a pharmacocluster. The methods are also applicable for classifying polypeptides, from a family of polypeptides that bind the same ligand, into pharmacofamilies based on bound conformations of the ligand. Accordingly, methods are provided for grouping polypeptides into pharmacofamilies by determining bound conformations of a ligand or a conformation-dependent property of a ligand independent of a determination of the structure of the polypeptide. An advantage of classifying polypeptides according to bound conformations of a ligand is that a pharmacofamily is likely to contain polypeptides having greater binding specificity for a particular molecule than other polypeptides in the same family. Thus, the methods allow identification of a pharmacofamily that can specifically interact with a particular therapeutic agent or drug.

Additionally, the methods of the invention can be used to determine a conformer model or pharmacophore model based on a bound conformation or conformation-dependent property of a ligand bound to polypeptides in a pharmacofamily. The invention is therefore advantageous in providing a model for the design and identification of therapeutic compounds having specificity for a pharmacofamily of polypeptides.

Another advantage of the invention is that the methods provide a correlation between ligand conformation, a parameter that is relatively easy to measure, and polypeptide structure, a parameter of tremendous value but often difficult to measure. Therefore, the methods of the invention can be used to determine structural characteristics of a polypeptide based on a conformation-dependent property of a bound ligand.

As used herein, the term “pharmacocluster” refers to a collection of substantially the same bound conformations of a ligand, or portion thereof, bound to two or more polypeptides. A member conformation of a pharmacocluster can have (1) a conformation that is more similar to an average conformation of the members in its pharmacocluster than to any other pharmacocluster and (2) a conformation that is more similar to an average conformation of the members in its own pharmacocluster than the most similar average structures from different pharmacoclusters are to each other, wherein the pharmacoclusters consist of conformations of the same ligand or portion thereof. The pharmacocluster is determined for a ligand bound to different polypeptides but does not require that a structure of the polypeptide be known or included as part of a bound conformation of a ligand. A bound conformation of a ligand can include the entire ligand structure or selected atoms including a portion of the complete atomic composition of the ligand so long as the number of atoms provides sufficient information to distinguish one pharmacocluster from another. A pharmacocluster can include both the bound conformations of a ligand, or portion thereof, and one or more atoms that both interact with the ligand and are from a bound polypeptide. Thus, a pharmacocluster can include conformational information of 1 or more, 2 or more, 5 or more, 10 or more, 20 or more, 30 or more, 40 or more, 50 or more or 100 or more atoms of a ligand bound conformation.

Accordingly, portions of bound conformations of two or more different ligands can be included in a ligand pharmacocluster so long as the portions selected from each ligand have a core bound conformation that is substantially the same. A core bound conformation can consist of portions of bound conformations of ligands wherein the portions have identical structural formula and conformation. A core bound conformation can also consist of portions of bound conformations of ligands wherein the portions have different structural formulas so long as the portions have substantially the same conformation. The structural formula, as it is understood in the art, is a 2 dimensional representation of a molecule that identifies the atoms and covalent bonds between each atom in the molecule. The structural formula does not necessarily include information sufficient to determine conformation of a molecule. For example, a common structural formula representation of cyclohexane can be a hexagon with 2 hydrogens attached to each carbon being in equivalent positions. However, a stable conformation of cyclohexane in solution may appear as a “chair” or “boat” shape with hydrogens in either axial or equitorial positions relative to the molecular plane.

As used herein, the term “conformation-dependent property,” when used in reference to a ligand, refers to a characteristic of a ligand that specifically correlates with the three dimensional structure of a ligand or the orientation in space of selected atoms and bonds of the ligand. Thus, a ligand bound to a polypeptide in a distinct conformation will have at least one unique conformation-dependent property correlated with the bound conformation of the ligand. A conformation-dependent property can be derived from or include the entire ligand structure or selected atoms and bonds, including a fragment or portion of the complete atomic composition of the ligand. A conformation-dependent property that includes selected atoms and bonds of a ligand can include 2 or more, 3 or more, 5 or more, 10 or more, 15 or more, 20 or more, 25 or more, or 50 or more atoms of a bound conformation of a ligand.

A characteristic that specifically correlates with a three dimensional structure of a ligand is a characteristic that is substantially different between at least two different bound conformations of the same ligand and, therefore, distinguishes the two different bound conformations. A conformation-dependent property can include a physical or chemical characteristic of a ligand, for example, absorption and emission of heat, absorption and emission of electromagnetic radiation, rotation of polarized light, magnetic moment, spin state of electrons, or polarity. A conformation-dependent property can also include a structural characteristic of a ligand based, for example, on an X-ray diffraction pattern or a nuclear magnetic resonance (NMR) spectrum. A conformation-dependent property can additionally include a characteristic based on a structural model, for example, an electron density map, atomic coordinates, or x-ray structure. A conformation-dependent property can include a characteristic spectroscopic signal based on, for example, Raman, circular dichroism (CD), optical rotation, electron paramagnetic resonance (EPR), infrared (IR), ultraviolet/visible absorbance (UW/Vis), fluorescence, or luminescence spectroscopies. A conformation-dependent property can also include a characteristic NMR signal, for example, chemical shift, J coupling, dipolar coupling, cross-correlation, nuclear spin relaxation, transferred nuclear Overhauser effect, or combinations thereof. A conformation-dependent property can additionally include a thermodynamic or kinetic characteristic based on, for example, calorimetric measurement or binding affinity measurement. Furthermore, a conformation-dependent property can include characteristic based on electrical measurement, for example, voltammetry or conductance.

As used herein, “selected” conformation-dependent properties are identified to form a set of conformation-dependent properties that can include, for example, the entire set of conformation-dependent properties associated with the bound conformations of a ligand in a pharmacocluster or a subset of conformation-dependent properties associated with the bound conformations of a ligand in a pharmacocluster, so long as the subset of conformation-dependent properties are sufficient to identify a unique conformation of the ligand. A selected conformation-dependent property can include any of the above described properties, for example, a physical or chemical property, structural data, a structural model, a spectroscopic signal, a thermodynamic or kinetic measurement or an electrical measurement.

As used herein, the term “bound conformation,” when used in reference to a ligand, refers to the location of atoms of a ligand relative to each other in three dimensional space, where the ligand is bound to a polypeptide. The location of atoms in a ligand can be described, for example, according to bond angles, bond distances, relative locations of electron density, probable occupancy of atoms at points in space relative to each other, probable occupancy of electrons at points in space relative to each other or combinations thereof.

As used herein, a “selected” bound conformation refers to a set of bound conformations that can include, for example, the entire set of defined bound conformations or a subset of bound conformations of a ligand.

As used herein, the term “clustering” refers to assigning related bound conformations of a ligand, or portion thereof, into a first collection such that the conformations residing in the first collection can be overlaid with substantial overlap and bound conformations from two different collections cannot be overlaid with a better overlap than that resulting from members of the first collection. Exemplary clustering of ligand conformations are disclosed herein (see Example I).

As used herein, the term “ligand” refers to a molecule that can specifically bind to a polypeptide. Specific binding, as it is used herein, refers to binding that is detectable over non-specific interactions by quantifiable assays well known in the art. A ligand can be essentially any type of natural or synthetic molecule including, for example, a polypeptide, nucleic acid, carbohydrate, lipid, amino acid, nucleotide or any organic derived compound. The term also encompasses a cofactor or a substrate of a polypeptide having enzymatic activity, or substrate that is inert to catalytic conversion by the bound polypeptide. Specific binding to a polypeptide can be due to covalent or non covalent interactions.

As used herein, the term “bound to two or more polypeptides,” when used in reference to a ligand is intended to refer to two or more complexes consisting of a ligand and a polypeptide. A complex can include, for example, a single ligand bound to a single polypeptide. A complex can also include a single ligand bound to more than one polypeptides including, for example, a complex in which a ligand is bound at the interface of interacting polypeptides. A complex can also include multiple ligands, however, conformation dependent properties of all ligands of the complex need not be identified. A complex results from a specific interaction between a polypeptide and a ligand.

As used herein, the term “substantially the same,” when used in reference to bound conformations of a ligand, or portion thereof, is intended to refer to two or more bound conformations that can be overlaid upon each other in 3 dimensional space such that all corresponding atoms between the two conformations are overlapped. Accordingly, “substantially different” bound conformations cannot be overlaid upon each other in 3-dimensional space such that all corresponding atoms between the two bound conformations are overlapped.

As used herein, the term “polypeptide” is intended to refer to a peptide polymer of two or more amino acids. The term is similarly intended to include polymers containing amino acid sterioisomers, analogues and functional mimetics thereof. For example, derivatives can include chemical modifications of amino acids such as alkylation, acylation, carbamylation, iodination, or any modification which derivatizes the polypeptide. Analogues can include modified amino acids, for example, hydroxyproline or carboxyglutamate, and can include amino acids, or analogs thereof, that are not linked by peptide bonds. Mimetics encompass chemicals containing chemical moieties that mimic the function of the polypeptide regardless of the predicted three-dimensional structure of the compound. For example, if a polypeptide contains two charged chemical moieties in a functional domain, a mimetic places two charged chemical moieties in a spatial orientation and constrained structure so that the corresponding charge is maintained in three-dimensional space. Thus, all of these modifications are included within the term “polypeptide” so long as the polypeptide retains its binding function.

As used herein, the term “root mean square deviation,” or RMSD, refers to a standard deviation which quantifies the structural variability in a population of bound conformations of a ligand. The term is intended to be consistent with its meaning as understood in the art as described for example in Doucet and Weber, Computer-Aided Molecular Design: Theory and Applications, Academic Press, San Diego Calif. (1996).

As used herein, the term “family,” when used in reference to characterizing polypeptides having ligand binding activity, is intended to refer to polypeptides that can bind to the same ligand, or portion thereof. A polypeptide family can contain polypeptides having binding activity for a common ligand with sufficient affinity, avidity or specificity to allow measurement of the binding event. As defined herein a “member” of a polypeptide family refers to an individual polypeptide that can be classified in a polypeptide family because the polypeptide binds a ligand, or portion thereof, that binds another polypeptide in a polypeptide family. The bound conformations of a ligand bound by individual members of a family can be substantially the same or different from each other.

As used herein, the term “pharmacofamily,” when used in reference to polypeptides, is intended to refer to polypeptides that can be classified together in a population because they individually bind a ligand such that the ligand is bound in substantially the same conformation. As defined herein a “member” of a polypeptide pharmacofamily refers to an individual polypeptide that is classified in a polypeptide pharmacofamily because the polypeptide binds a conformation of a ligand that is substantially the same as a conformation of the ligand bound to another polypeptide in the pharmacofamily.

As used herein, the term “grouping” refers to assigning related polypeptides into a family or pharmacofamily such that the polypeptide members of a family bind the same ligand and the polypeptide members of a pharmacofamily bind substantially the same bound conformation of a ligand.

As used herein, the term “fold,” when used in reference to a polypeptide, refers to a specific geometric arrangement and connectivity of a combination of secondary structure elements in a polypeptide structure. Secondary structure elements of a polypeptide that can be arranged into a fold including, for example, alpha helices, beta sheets, turns and loops are well known in the art. Folds of a polypeptide can be recognized by one skilled in the art and are described in, for example, Branden and Tooze, Introduction to protein structure, Garland Publishing, New York (1991) and Richardson, Adv. Prot. Chem. 34:167-339 (1981).

As used herein, “modeling the three dimensional structure” when used in reference to a polypeptide refers to determining a conformation for a polypeptide. A conformation of a polypeptide can be determined, for example, from empirical data specifying structure or from a compared conformation used as a template. A conformation can be determined at any desired level of resolution sufficient to identify, for example, overall shape of a polypeptide, tertiary structure elements, secondary structure elements, polypeptide backbone structure, amino acid residue identity or location of individual atoms.

As used herein, the term “structural model,” when used in reference to a polypeptide, refers to a representation of a 3 dimensional structure of a polypeptide. A structural model can be determined from empirical data derived from, for example, X-ray crystallography or nuclear magnetic resonance spectroscopy. A structural model can also be derived from a theoretical calculation including, for example, comparison to a known structure or ab initio molecular modeling. A representation of a structural model can include, for example, an electron density map, atomic coordinates, x-ray structure model, ball and stick model, density map, space filling model, surface map, Connolly surface, Van der Waals surface or CPK model.

As used herein, the term “conformer model” refers to a representation of points in a defined coordinate system wherein a point corresponds to a position of an atom in a bound conformation of a ligand. The coordinate system is preferably in 3 dimensions, however, manipulation or computation of a model can be performed in 2 dimensions or even 4 or more dimensions in cases where such methods are preferred. A point in the representation of points can, for example, correlate with the center of an atom. Additionally, a point in the representation of points can be incorporated into a line, plane or sphere to include a shape of one or more atom or volume occupied by one or more atom. A conformer model can be derived from 2 or more bound conformations of a ligand. For example a conformer model can be generated from 3 or more, 4 or more, 5 or more, 6 or more, 7 or more, 8 or more, 10 or more, 15 or more, 20 or more or 25 or more bound conformations of a ligand.

As used herein, the term “average structure,” when used in reference to bound conformations of a ligand in a pharmacocluster, refers to conformer model, derived by superimposing the bound conformations of a ligand in a pharmacocluster, and determining an average location in space for corresponding atoms.

As used herein, the term “pharmacophore model” refers to a representation of points in a defined coordinate system wherein a point corresponds to a position or other characteristic of an atom or chemical moiety in a bound conformation of a ligand and/or an interacting polypeptide or ordered water. An ordered water is an observable water in a model derived from structural determination of a polypeptide. A pharmacophore model can include, for example, atoms of a bound conformation of a ligand, or portion thereof. A pharmacophore model can include both the bound conformations of a ligand, or portion thereof, and one or more atoms that both interact with the ligand and are from a bound polypeptide. Thus, in addition to geometric characteristics of a bound conformation of a ligand, a pharmacophore model can indicate other characteristics including, for example, charge or hydrophobicity of an atom or chemical moiety. A pharmacaphore model can incorporate internal interactions within the bound conformation of a ligand or interactions between a bound conformation of a ligand and a polypeptide or other receptor including, for example, van der Waals interactions, hydrogen bonds, ionic bonds, and hydrophobic interactions. A pharmacophore model can be derived from 2 or more bound conformations of a ligand. For example a conformer model can be generated from 3 or more, 4 or more, 5 or more, 6 or more, 7 or more, 8 or more, 10 or more, 15 or more, 20 or more or 25 or more bound conformations of a ligand.

A point in a pharmacophore model can, for example, correlate with the center of an atom or moiety. Additionally, a point in the representation of points can be incorporated into a line, plane or sphere to indicate a characteristic other than a center of an atom or moiety including, for example, shape of an atom or moiety or volume occupied by an atom or moiety. The coordinate system of a pharmacophore model is preferably in 3 dimensions, however, manipulation or computation of a model can be performed in 2 dimensions or even 4 or more dimensions in cases where such methods are preferred. Multidimensional coordinate systems in which a pharmacophore model can be represented include, for example, Cartesian coordinate systems, fractional coordinate systems, or reciprocal space. The term pharmacophore model is intended to encompass a conformer model.

As used herein, the term “moiety” refers to a group of atoms that form a part or portion of a larger molecule. A moiety can consist of any number of atoms in a portion of a ligand and can correlate with a physical or chemical property conferred upon the ligand by the combined atoms. Exemplary moieties of a nicotinamide adenine dinucleotide ligand include a phosphate, nicotinamide ring, amino group, amide group or ribose ring. In addition, a nicotinamide adenine dinucleotide group can be a moiety. For example, a nicotinamide adenine dinucleotide can be a moiety of the 2′P phosphate in a nicotinamide adenine dinucleotide phosphate molecule (see FIG. 2 for location of the 2′P phosphate in nicotinamide adenine dinucleotide phosphate).

The invention provides a method for identifying a pharmacocluster. The method includes the steps of (a) determining bound conformations of a ligand bound to different polypeptides, and (b) clustering two or more bound conformations of the ligand having substantially the same bound conformation, thereby identifying a pharmacocluster. The invention also provides a method for identifying a member of a pharmacocluster. The method includes the steps of (a) determining a bound conformation of a ligand bound to a polypeptide; and (b) determining a pharmacocluster having substantially the same bound conformation as the bound conformation, thereby identifying the bound conformation of the ligand as a member of the pharmacocluster.

A bound conformation of a ligand bound to a polypeptide can be determined from a previously observed molecular structure or from data specifying a molecular structure for a bound conformation of a ligand. Previously observed structures can be acquired for use in the invention by searching a database of existing structures. An example of a database that includes structures of bound conformations of ligands bound to polypeptides is the Protein Data Bank (PDB, operated by the Research Collaboratory for Structural Bioinformatics, see Berman et al., Nucleic Acids Research, 28:235-242 (2000)). A database can be searched, for example, by querying based on chemical property information or on structural information. In the latter approach, an algorithm based on finding a match to a template can be used as described, for example, in Martin, “Database Searching in Drug Design,” J. Med. Chem. 35:2145-2154 (1992).

A bound conformation of a ligand bound to a polypeptide can be determined from an empirical measurement, or from a database. Data specifying a structure can be acquired using any method available in the art for structural determination of a ligand bound to a polypeptide. For example, X-ray crystallography can be performed with a crystallized complex of a polypeptide and ligand to determine a bound conformation of the ligand bound to the polypeptide. Methods for obtaining such crystal complexes and determining structures from them are well known in the art as described for example in McRee et al., Practical Protein Crystallography, Academic Press, San Diego 1993; Stout and Jensen, X-ray Structure Determination: A practical guide, 2^{nd} Ed. Wiley, New York (1989); and McPherson, The Preparation and Analysis of Protein Crystals, Wiley, New York (1982). Another method useful for determining a bound conformation of a ligand bound to a polypeptide is Nuclear Magnetic Resonance (NMR). NMR methods are well known in the art and include those described for example in Reid, Protein NMR Techniques, Humana Press, Totowa N.J. (1997); and Cavanaugh et al., Protein NMR Spectroscopy: Principles and Practice, ch. 7, Academic Press, San Diego Calif. (1996).

A bound conformation of a ligand can also be determined from a hypothetical model. For example, a hypothetical model of a bound conformation of a ligand can be produced using an algorithm which docks a ligand to a polypeptide of known structure and fits the ligand to the polypeptide binding site. Algorithms available in the art for fitting a ligand structure to a polypeptide binding site include, for example, DOCK (Kuntz et al., J. Mol. Biol. 161:269-288 (1982)) and INSIGHT98 (Molecular Simulations Inc., San Diego, Calif.).

A molecular structure can be conveniently stored and manipulated using structural coordinates. Structural coordinates can occur in any format known in the art so long as the format can provide an accurate reproduction of the observed structure. For example, crystal coordinates can occur in a variety of file types including, for example, .fin, .df, .phs, or .pdb as described for example in McRee, supra. Although the examples above describe structural coordinates derived from X-ray crystallographic analysis or NMR spectroscopy, one skilled in the art will recognize that structural coordinates can be derived from any method known in the art to determine a bound conformation of a ligand bound to a polypeptide.

Structures at atomic level resolution can be useful in the methods of the invention. Resolution, when used to describe molecular structures, refers to the minimum distance that can be resolved in the observed structure. Thus, resolution where individual atoms can be resolved is referred to in the art as atomic resolution. Resolution is commonly reported as a numerical value in units of Angstroms (Å, 10⁻¹ meter) correlated with the minimum distance which can be resolved such that smaller values indicate higher resolution. Bound conformations of a ligand useful in the methods of the invention can have a resolution better than about 10 Å, 5 Å, 3 Å, 2.5 Å, 2.0 Å, 1.5 Å, 1.0 Å, 0.8 Å, 0.6 Å, 0.4 Å, or about 0.2 Å or better. Resolution can also be reported as an all atom RMSD as used, for example, in reporting NMR data. Bound conformations of a ligand useful in the methods of the invention can have an all atom RMSD better than about 10 Å, 5 Å, 3 Å, 2.5 Å, 2.0 Å, 1.5 Å, 1.0 Å, 0.8 Å, 0.6 Å, 0.4 Å, or about 0.2 Å or better.

An advantage of the methods of the invention is that a structure of a polypeptide bound to a bound conformation of a ligand need not be determined to identify a pharmacocluster. Thus, methods that detect only the structure of the ligand can be used in the invention. In some cases determination or refinement of only the structure of the ligand in a polypeptide-ligand complex will be required. In addition, methods that detect a conformation-dependent property of the ligand can be used to identify a pharmacocluster. Methods that can be used to determine a conformation-dependent property of a ligand in a polypeptide-ligand complex without determining the structure of the polypeptide include, for example, Electron Nuclear Double Resonance spectroscopy (ENDOR, as described in Van Doorslaer and Schweiger, Naturwissenschaften 87:245-55(2000)), Electron Paramagnetic Resonance spectroscopy (EPR, described in Cantor and Schimmel Biophysical Chemistry, Part I: The conformation of biological macromolecules W. H. Freeman and Company (1980)), chemically induced dynamic nuclear polarization (CIDNP, described in Siebert et al., Glycoconj J.14:945-9 (1997) and Consonni et al., FEBS Lett. 372:135-9 (1995)), solid state NMR (described in Mehring, M. High Resolution NMR spectroscopy in Solids,2^{nd} ed. Springer-Verlag, Berlin (1983) and liquid phase NMR (described in Wuthrich, NMR of Proteins and Nucleic Acids John Wiley & Sons, Inc. (1986)). Thus, the invention can be performed in a manner whereby the time and cost associated with a full determination of a polypeptide structure is avoided.

Any representation that correlates with the structure of a bound conformation of a ligand can be used in the methods of the invention. For example, a convenient and commonly used representation is a displayed image of the structure. Displayed images that are particularly useful for determining the bound conformation of a ligand bound to polypeptides include, for example, ball and stick models, density maps, space filling models, surface map, Connolly surfaces, Van der Waals surfaces or CPK model. Display of images as a computer output, for example, on a video screen can be advantageous as described below.

Clustering can be performed with any ligand or any number of bound conformations of a ligand. The methods of the invention can be performed by clustering 2 or more bound conformations of a ligand. For example, clustering can be performed with 3 or more, 4 or more, 5 or more, 6 or more, 7 or more, 8 or more, 9 or more, 10 or more, 11 or more, 12 or more, 13 or more, 14 or more, 15 or more or 20 or more bound conformations of a ligand. The methods of the invention can be used with any number bound conformations of a ligand. Due to the large sizes of data sets required to represent bound conformations of a ligand, methods of clustering bound conformations are generally performed on a computer. The methods are compatible with any computer that can support molecular modeling software including for example a personal computer, silicon graphics workstation, or supercomputer. A variety of computer software programs are available for molecular modeling including, for example, GRASP (Nicholls, A., supra), ALADDIN (Van Drie et al. supra), INSIGHT98 (Molecular Simulations Inc., San Diego Calif.), RASMOL (Sayle et al., Trends Biochem Sci. 20:374-376 (1995)) and MOLMOL (Koradi et al., J. Mol. Graphics 14:51-55 (1996 )).

Once a bound conformation of a ligand bound to different polypeptides has been determined, two or more bound conformations of the ligand can be compared and those having substantially the same bound conformation can be clustered. Methods of comparison include, for example, a method that provides alignment of two or more bound conformations of a ligand and evaluation of the degree of overlap in the two structures. Methods of comparison can be performed in an iterative fashion until a best fit is identified.

Methods of comparing bound conformations of bound ligands include, for example, cluster analysis, visual inspection and pairwise structural comparisons. Cluster analysis is commonly performed by, but not limited to, partitioning methods or hierarchical methods as described, for example, in Kauffman and Rousseeuw, Finding Groups in Data: An Introduction to Cluster Analysis, John Wiley and Sons Inc., New York (1990). Partitioning methods that can be used include, for example, partitioning around mediods, clustering large applications, and fuzzy analysis, as described in Kauffman and Rousseeuw, supra. Hierarchical methods useful in the invention include, for example, agglomerative nesting, divisive analysis, and monothetic analysis, as described in Kauffman and Rousseeuw, supra. Algorithms for cluster analysis of molecular structures are known in the art and include, for example, COMPARE (Chiron Corp, 1995; distributed by Quantum Chemistry program Exchange, Indianapolis Ind.). COMPARE can be used to make all possible pairwise comparisons between a set of conformations of the same ligand(s). COMPARE reads PDB files and uses a Ferro-Hermanns ORIENT algorithm for a least squares root mean square (RMS) fit. The structures can be clustered into groups using the Jarvis-Patrick nearest neighbors algorithm. Based on the RMS deviation between ligand conformers, a list of ‘nearest neighbors’ for each conformer are generated. Two conformers are then grouped together or clustered if: (1) the RMS deviation is sufficiently small and (2) if both conformers share a determined number of common ‘neighbors’. Both criteria are adjusted by the program to generate clusters based on a user defined cutoff for distance between individual clusters. Follow up analysis was conducted using InsightII to verify clusters. A member conformation is identified as being closer to the averaged coordinates of conformations within its family than to the averaged coordinates of any other family.

Using methods such as those described above, one skilled in the art will know how to identify conformations that are substantially the same. For example, similarity can be evaluated according to the goodness of fit between two or more bound conformations of a ligand. Goodness of fit can be represented by a variety of parameters known in the art including, for example, the root mean square deviation (RMSD). A lower RMSD between structures correlates with a better fit compared to a higher RMSD between structures. Bound conformations of a ligand having substantially the same conformations can be identified by comparing mean RMSD values within and between pharmacoclusters. Accordingly, bound conformations of a ligand having substantially the same conformations can have a mean RMSD compared to an average structure for the pharmacocluster that is less than 1.1 Å. Two or more bound conformations of a ligand can be clustered by assigning bound conformations of a ligand into a collection such that the conformations of a ligand residing in the collection are substantially the same. Members of a pharmacocluster can also be identified as having RMSD values compared to an average structure for the pharmacocluster that are less than 1.0 Å, 0.9 Å, 0.8 Å, 0.7 Å, 0.6 Å, 0.5 Å, 0.4 Å, 0.3 Å, 0.2 Å or 0.1 Å.

A bound conformation of a ligand that is a member of a pharmacocluster can also be identified by comparing the RMSD for the bound conformation to an average conformation of the members in multiple pharmacoclusters. Using this value for comparison, a member conformation is identified as having a smaller RMSD when compared to the averaged coordinates of conformations within its family than when compared to the averaged coordinates of any other family. In addition, a member of a pharmacocluster can be identified as having an RMSD compared to an average conformation of the members in a pharmacocluster that is smaller than the RMSD between each family's average coordinates. For example, as described in Example I, RMSD values for members of pharmacoclusters 1-8 as presented in Tables 3A, 4A, 5A, 6A, 7A, 8A, 9A or 10A, respectively, can be compared to RMSD values between each pharmacocluster as presented in Table 2. Comparisons similar to those described above can be made for bound conformations of any ligand according to the methods described in the Examples.

In addition, bound conformations of a ligand can be compared with respect to dihedral angles at particular bonds. Exemplary methods for comparing dihedral angles between pharmacoclusters is described in Example I and Table 1. Comparison between dihedral angles can be used, for example, in combination with overall RMSD comparisons such as those described above. Therefore, bound conformations that are not easily distinguished by comparison of overall RMSD alone, can be distinguished according to the combined comparison of RMSD and dihedral angle. Bound conformations of a ligand that are members of different pharmacoclusters can have dihedral angles that differ, for example, by at least about 10 degrees, 30 degrees, 45 degrees, 90 degrees or 180 degrees.

The invention also provides a pharmacocluster selected from the cluster consisting of pharmacocluster 1, pharmacocluster 2, pharmacocluster 3, pharmacocluster 4, pharmacocluster 5, pharmacocluster 6, pharmacocluster 7, and pharmacocluster 8 correlated with the pharmacofamilies listed in Table 11.

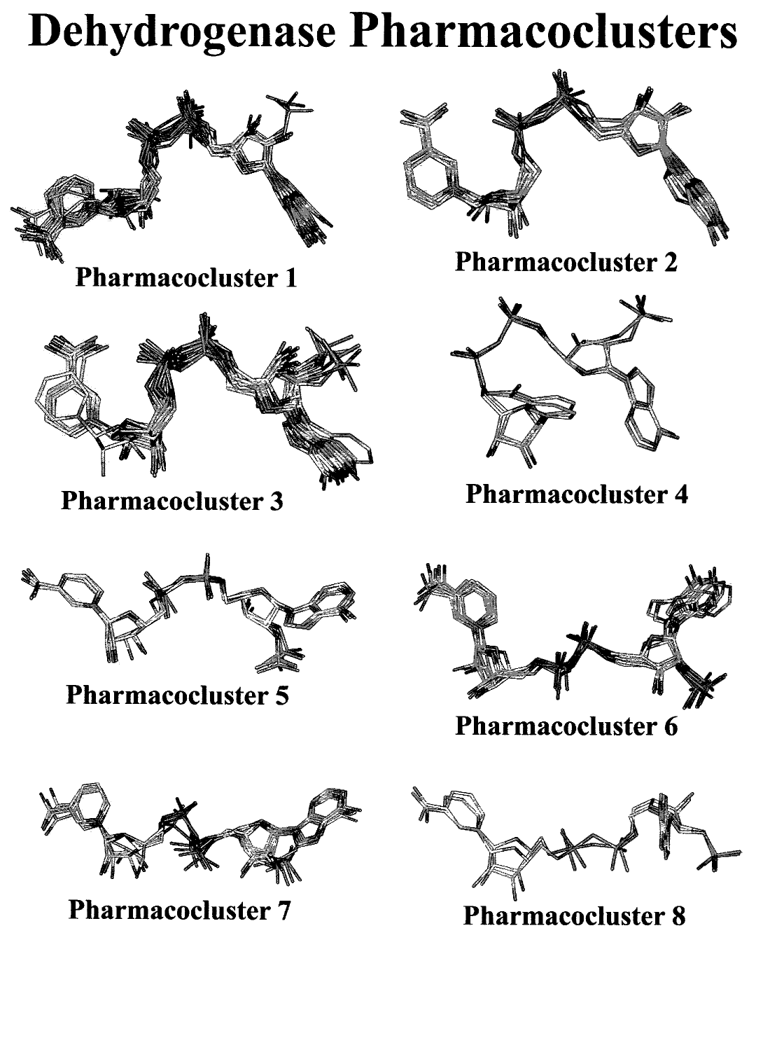

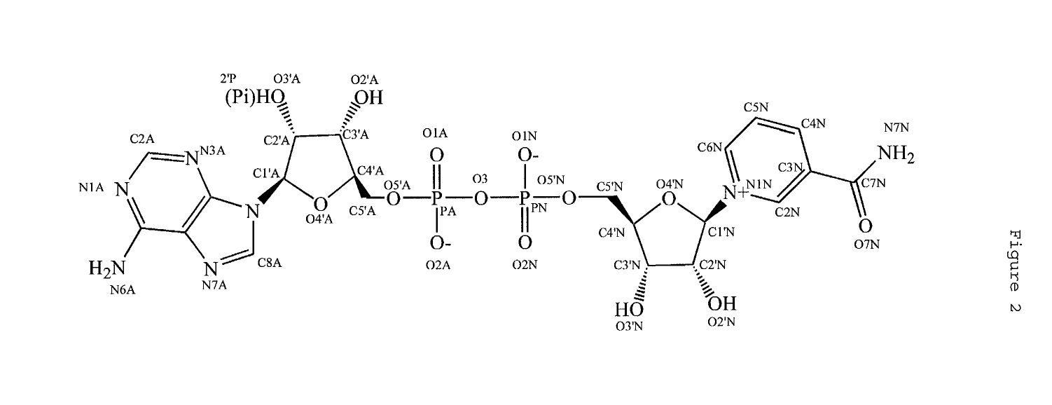

Pharmacoclusters 1 through 8 contain bound conformations of NAD(P)(H) determined from structures deposited in the PDB for NAD(P)(H) bound to oxidoreductase polypeptides. Pharmacoclusters are shown in FIG. 1 and described in further detail in Example I. The pharmacoclusters of FIG. 1 display substantial overlap between bound conformations of NAD(P)(H) within the cluster, as can be identified by visual inspection of the structures. Quantitative comparison of the bound conformations in each pharmacocluster demonstrates that each pharmacocluster displays less than about 1.1 Å difference in RMSD between each conformation of NAD(P)(H) and the average bound conformation for the respective pharmacocluster as described in Example I.

Pharmacoclusters can be used to identify a ligand having specificity for one or more polypeptide pharmacofamilies (see Example V). As described herein, a pharmacophore model or conformer model can be derived from one or more cluster. These models can be used to identify a ligand having specificity for one or more pharmacofamilies of oxidoreductases, for example, by using the model to query a database of molecules for a potential ligand or by using the model to guide in the design of a synthetic ligand. An example of using a pharmacophore of the invention to identify a binding compound is provided in Example VI.

Pharmacoclusters, including, for example, pharmacoclusters 1 through 8 can also be used to identify a new polypeptide member of a polypeptide pharmacofamily. Using the methods described herein, for example, a pharmacocluster can be used to produce a pharmacophore model or conformer model to which a bound conformation of a ligand can be compared. A polypeptide bound to a bound conformation of a ligand that is similar to the model can be classified into an appropriate polypeptide pharmacofamily based on this comparison. By a similar method, a bound conformation of a ligand can be directly compared to a pharmacocluster to classify the polypeptide bound to the conformation of a ligand into an appropriate pharmacofamily.

The methods of the invention can also be used with a portion of a bound conformation of a ligand to identify a pharmacocluster. The method consists of (a) determining a bound conformation of a ligand, or portion thereof, bound to two or more polypeptides, and (b) clustering two or more bound conformations of the ligand, or portion thereof having substantially the same bound conformation, thereby identifying a pharmacocluster.

A bound conformation of a portion of a ligand can include selected atoms and/or bonds of a ligand and can include, for example, a continuous sequence of atoms and/or bonds or a discontinuous sequence of selected atoms and/or bonds that, when described independent of the complete ligand structure, may not appear to be attached to each other. Such a portion can include 2 or more atoms of a bound conformation of a ligand or 3 or more, 4 or more, 5 or more, 6 or more, 7 or more, 8 or more, 9 or more, 10 or more, 15 or more, 20 or more, 25 or more or 50 or more atoms of a bound conformation of a ligand. A bound conformation of a portion of a ligand bound to a polypeptide can be identified according to the same methods described above for identifying a bound conformation of a ligand bound to a polypeptide. Two or more bound conformations of a portion of a ligand can be clustered as described above so long as the bound conformations that are clustered correspond to bound portions of the ligand having the same structural formula. For example, in a case where determination of the complete structure of a ligand has not been achieved, a complete structure of a ligand has not been achieved, a bound conformation of a portion of the ligand corresponding to the structurally determined portion can be used in the methods of the invention.

A pharmacocluster can include portions of bound conformations derived from different ligands so long as the portions have a core bound conformation that is substantially the same. For example, portions having the same structural formula and bond configuration can share a core bound conformation. The bond configuration describes the relative position of atoms attached to a chiral atom of a ligand. Accordingly, R and S sterioisomers of a chiral atom have different bond configurations. Other terms used in the art to designate different bond configurations include, for example, cis and trans configurations of atoms attached to carbons that are double bonded, or Z and E configurations of atoms attached to carbons that are double bonded. An example of portions of ligands having the same structural formula and bond configuration that can share a core bound conformation are the nicotinamide adenine dinucleotide portions of nicotinamide adenine dinucleotide phosphate (NADP) and nicotinamide adenine dinucleotide (NAD). Additionally, portions of ligands having different charge, atom substitution or bond hybridization can share a core bound conformation. An example of portions of ligands having different charge and bond hybridization that can share a core bound conformation are the nicotinamide adenine dinucleotide portions of oxidized nicotinamide adenine dinucleotide (NAD) and reduced nicotinamide adenine dinucleotide (NADH). In cases where the core structures of two ligands bind with substantially the same conformation to polypeptides, the core bound conformations can be clustered according to the methods of the invention (see Example I).

Substantially the same bound conformation of a portion of a bound conformation of a ligand, including non-continuous atoms, can be identified according to the root mean square deviation and compared directly. Conformations of portions having different numbers of atoms can also be compared via root mean square deviation per equivalent atom (RMSD/N, where N is the number of atoms compared). A lower value of RMSD/N indicates increased similarity between the two or more bound ligand conformations that are clustered. One skilled in the art will know that RMSD/N has a compensational origin and consideration of the effect of N is required for comparison of RMSD/N between pharmacoclusters having different values of N. For example, the lower the value of RMSD/N the lower should be the value of N to indicate substantial similarity.

The invention can be used with any ligand for which bound conformations of the ligand bound to different polypeptides can be determined including, for example, chemical or biological molecules such as simple or complex organic molecules, metal-containing compounds, carbohydrates, peptides, peptidomimetics, carbohydrates, lipids, nucleic acids, and the like.

In one embodiment, the compositions and methods of the invention can be used with a ligand that is a nucleotide derivative including, for example, a nicotinamide adenine dinucleotide-related molecule. Nicotinamide adenine dinucleotide-related (NAD-related) molecules that can be used in the methods of the invention can be selected from the group consisting of oxidized nicotinamide adenine dinucleotide (NAD⁺), reduced nicotinamide adenine dinucleotide (NADH), oxidized nicotinamide adenine dinucleotide phosphate (NADP⁺), and reduced nicotinamide adenine dinucleotide phosphate (NADPH). An NAD-related molecule can also be a mimetic of the above- described molecules. Use of a NAD-related molecule to identify pharmacoclusters is described in Example I.

A mimetic is a molecule that has at least one function that is substantially the same as a function of a second molecule. A mimetic of a ligand can be identified according to its ability to bind to the same sites on a polypeptide as the ligand. For example, a mimetic can be identified by a binding competition assay using a ligand and a mimetic. The structure of a mimetic can be similar or different compared to the structure of the second molecule. The term can encompass molecules having portions similar to corresponding portions of the ligand in terms of structure or function.

Examples of mimetics to the common ligand NADH, for example cibacron blue, are described in Dye-Ligand Chromatography, Amicon Corp., Lexington Mass. (1980). Numerous other examples of NADH-mimics, including useful modifications to obtain such mimics, are described in Everse et al. (eds.), The Pyridine Nucleotide Coenzymes, Academic Press, New York N.Y. (1982). Particular analogs include nicotinamide 2-aminopurine dinucleotide, nicotinamide 8-azidoadenine dinucleotide, nicotinamide 1-deazapurine dinucleotide, 3-aminopyridine adenine dinucleotide, 3-acetyl pyridine adenine dinucleotide, thiazole amide adenine dinucleotide, 3-diazoacetylpyridine adenine dinucleotide and 5-aminonicotinamide adenine dinucleotide. Particular mimetics can be identified and selected by ligand-displacement assays, for example using competitive binding assays with a known ligand as is well known in the art. Mimetic candidates can also be identified by searching databases of compounds for structural similarity with the common ligand or a mimetic.

In another embodiment, the methods of the invention can be used with a ligand that is an adenosine phosphate-related molecule. Adenosine phosphate-related molecules can be selected from the group consisting of adenosine triphosphate (ATP), adenosine diphosphate (ADP), adenosine monophosphate (AMP), and cyclic adenosine monophosphate (cAMP). An adenosine phophate-related molecule can also be a mimetic of the above-described molecules. A mimetic of an adenosine phosphate-related molecule that can be used in the invention includes, for example, quercetin, adenylylimidodiphosphate (AMP-PNP) or olomoucine.

A ligand useful in the methods of the invention can be a cofactor, coenzyme or vitamin including, for example, NAD, NADP, or ATP as described above. Other examples include thiamine (vitamin B₁), riboflavin (vitamin B₂), pyridoximine (vitamin B₆), cobalamin (vitamin B₁₂), pyrophosphate, flavin adenine dinucleotide (FAD), flavin mononucleotide (FMN), pyridoxal phosphate, coenzyme A, ascorbate (vitamin C), niacin, biotin, heme, porphyrin, folate, tetrahydrofolate, nucleotide such as guanosine triphosphate, cytidine triphosphate, thymidine triphosphate, uridine triphosphate, retinol (vitamin A), calciferol (vitamin D₂), ubiquinone, ubiquitin, α-tocopherol (vitamin E), farnesyl, geranylgeranyl, pterin, pteridine or S-adenosyl methionine (SAM).

A polypeptide can be used as a ligand in the invention. For example, a ligand can be a naturally occurring polypeptide ligand such as a ubiquitin or polypeptide hormone including, for example, insulin, human growth hormone, thyrotropin releasing hormone, adrenocorticotropic hormone, parathyroid hormone, follicle stimulating hormone, thyroid stimulating hormone, luteinizing hormone, human chorionic gonadotropin, epidermal growth factor, nerve growth factor and the like. In addition a polypeptide ligand can be a non-naturally occurring polypeptide that has binding activity. Such polypeptide ligands can be identified, for example, by screening a synthetic polypeptide library such as a phage display library or combinatorial polypeptide library as described below. A polypeptide ligand can also contain amino acid analogs or derivatives such as those described below. Methods of isolation of a polypeptide ligand are well known in the art and are described, for example, in Scopes, Protein Purification: Principles and Practice, 3^{rd} Ed., Springer-Verlag, New York (1994); Duetscher, Methods in Enzymology, Vol 182, Academic Press, San Diego (1990); and Coligan et al., Current protocols in Protein Science, John Wiley and Sons, Baltimore, Md. (2000).

A nucleic acid can also be used as a ligand in the invention. Examples of nucleic acid ligands useful in the invention include DNA, such as genomic DNA or cDNA or RNA such as mRNA, ribosomal RNA or tRNA. A nucleic acid ligand can also be a synthetic oligonucleotide. Such ligands can be identified by screening a random oligonucleotide library for ligand binding activity, for example, as described below. Nucleic acid ligands can also be isolated from a natural source or produced in a recombinant system using well known methods in the art including, for example, those described in Sambrook et al., Molecular Cloning: A Laboratory Manual, 2nd ed., Cold Spring Harbor Press, Plainview, New York (1989); Ausubel et al., Current Protocols in Molecular Biology (Supplement 47), John Wiley & Sons, New York (1999).

A ligand used in the invention can be an amino acid, amino acid analog or derivatized amino acid. An amino acid ligand can be one of the 20 essential amino acids or any other amino acid isolated from a natural source. Amino acid analogs useful in the invention include, for example, neurotransmitters such as gamma amino butyric acid, serotonin, dopamine, or norepenephrine or hormones such as thyroxine, epinephrine or melatonin. A synthetic amino acid, or analog thereof, can also be used in the invention. A synthetic amino acid can include chemical modifications of an amino acid such as alkylation, acylation, carbamylation, iodination, or any modification that derivatizes the amino acid. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine. Naturally occurring amino acid derivatives of the twenty standard amino acids can also be included in a cluster of bound conformations including, for example, 4-hydroxyproline, 5-hydroxylysine, 3-methylhistidine, homoserine, ornithine or carboxyglutamate.

A lipid ligand can also be used in the invention. Examples of lipid ligands include triglycerides, phospholipids, glycolipids or steroids. Steroids useful in the invention include, for example, glucocorticoids, mineralocorticoids, androgens, estrogens or progestins.

Another type of ligand that can be used in the invention is a carbohydrate. A carbohydrate ligand can be a monosaccharide such as glucose, fructose, ribose, glyceraldehyde, or erythrose; a disaccharide such as lactose, sucrose, or maltose; oligosaccharide such as those recognized by lectins such as agglutinin, peanut lectin or phytohemagglutinin, or a polysaccharide such as cellulose, chitin, or glycogen.

Methods for producing pluralities of compounds to use as ligands, including chemical or biological molecules such as simple or complex organic molecules, metal-containing compounds, carbohydrates, peptides, peptidomimetics, carbohydrates, lipids, nucleic acids, and the like, are well known in the art (see, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997), Gold et al., U.S. Pat Nos. 5,475,096 (1995), 5,789,157 (1998), and 5,270,163 (1993)). The advantage of using such a combinatorial library is that molecules do not have to be individually generated to identify a ligand that binds a polypeptide. Also, no prior knowledge of the exact characteristics of a binding polypeptide is required when using a combinatorial library. Libraries containing large numbers of natural and synthetic compounds also can be individually synthesized or obtained from commercial sources.

In addition, the invention provides a method for identifying a conformation-dependent property of a ligand. The method includes the steps of (a) determining bound conformations of a ligand bound to different polypeptides; (b) identifying two or more bound conformations of the ligand having substantially the same bound conformation, and (c) identifying a conformation-dependent property of the bound conformations of the ligand having substantially the same bound conformation, the conformation-dependent property being correlated with the bound conformation of the ligand.

A conformation-dependent property can be identified as any property that correlates with a bound conformation of a ligand such that a change in the bound conformation results in a change in the conformation-dependent property. Accordingly, a bound conformation of a ligand, or a portion thereof, can be a conformation-dependent property. A portion of a bound conformation of a ligand can be a contiguous fragment or a non-contiguous set of atoms or bonds. A bound conformation of a ligand, or portion thereof, can be identified by any method for determining the three dimensional structure of a ligand including as disclosed herein.

Other conformation-dependent properties include, for example, absorption and emission of heat, absorption and emission of electromagnetic radiation, rotation of polarized light, magnetic moment, spin state of electrons, or polarity, as disclosed herein, or other properties that can be identified as a spectroscopic signal. Methods known in the art for measuring changes in absorption and emission of heat that correlate with changes in bound conformation of a ligand include, for example, calorimetry. Methods known in the art for measuring changes in absorption and emission of electromagnetic radiation as they correlate with changes in bound conformation of a ligand include, for example, UV/VIS spectroscopy, fluorimetry, luminometry, infrared spectroscopy, Raman spectroscopy, resonance Raman spectroscopy, X-ray absorption fine structure spectroscopy (XAFS) and the like. A change in a bound conformation of a ligand that is correlated with a change in rotation of polarized light can be measured with circular dichroism spectroscopy or optical rotation spectroscopy. A change in magnetic moment or spin state of an electron that correlates with a change in a bound conformation can be measured, for example, with Electron paramagnetic resonance spectroscopy (EPR) or nuclear magnetic resonance spectroscopy (NMR).

When based on NMR data, a conformation-dependent property can be identified as an NMR signal including, for example, chemical shift, J coupling, dipolar coupling, cross-correlation, nuclear spin relaxation, transferred nuclear Overhauser effect, and any combination thereof. A conformation-dependent property can be identified by NMR methods in both fast and slow exchange regimes. For example, in many cases, the exchange rate of a complex between ligand and polypeptide is faster than the ligand spin relaxation rate (1/T_{1H}). In this situation, referred to as the “fast exchange regime,” transferred nuclear Overhauser effect (NOE) experiments can be performed to measure an intra-ligand proton-proton distance (Wuthrich, NMR of proteins and Nucleic Acids, Wiley, New York (1986) and Gronenborn, J. Magn. Res. 53:423-442 (1983)). Labeling of polypeptides is not required, and the ligand polypeptide concentration ratio can be adjusted to minimize line broadening of the ligand resonances while retaining strong NOE contribution from the bound form.

In a fast exchange regime, cross-correlated relaxation measurements can also provide structural information on ligand torsion angles (Carlomagno et al., J. Am. Chem Soc. 121:1945-1948 (1999)). These measurements include the ¹H-¹H dipole-dipole cross-correlation but can be extended to other cross-correlated relaxation mechanisms involving also homo- and heteronuclear chemical shielding anisotropy relaxation, as well as quadrupolar relaxation. For most of these heteronuclear experiments, the natural abundance of the isotope can be exploited. In cases where natural abundance of the isotope measured is not sufficient, isotope enriched ligands can be obtained from commercial sources such as Isotek (Miamisburg, Ohio) or Cambridge Isotope Laboratories (Andover, Mass.) or prepared by methods known in the art. Another method to determine a conformation-dependent property of a ligand in a fast exchange regime is use of residual homo- and heteronuclear dipolar couplings in partially aligned samples (Tolman et al. Proc. Natl. Acad. Sci. USA 92:9279-9283 (1995)).

In the slow exchange regime, the NMR signals arising from the bound conformation of the ligand are distinguished from those of the polypeptide to reduce resonance overlap. This can be achieved with different isotope labeling schemes of polypeptide, ligand or both. For large systems, perdeuteration of macromolecules and TROSY-type experiments (Pervushkin, Proc. Natl. Acad. Sci. USA 94:12366-12371 (1997)) can be used to minimize signal losses due to fast transverse relaxation of the resonances of the complex. With the appropriate sample requirements and isotope filtered experiments, cross-correlations, cross-relaxations and residual dipolar couplings can be measured and provide necessary structural information.

In addition, homo- and heteronuclear two and three bond J couplings can be obtained to provide information on torsion angles (Wuthrich, supra) . For example, as shown in Table 1 the bound conformations of NADP in pharmacocluster 4 and pharmacocluster 5 differ by a torsion angle defined by the atoms PN-O5′N-C5′N-C4′N (See FIG. 2 for atom labeling and bond location). Specifically, pharmacocluster 4 has a PN-O5′N-C5′N-C4′N torsion angle of 145 degrees and pharmacocluster 5 has a PN-O5′N-C5′N-C4′N angle of −112 degrees. These torsion angles can be measured and distinguished by measuring the three bond ³¹P-¹³C4′ J coupling constants that correspond to this torsion angle (Marino, Acc. Chem. Res. 32:614-623 (1999)). Basically, two ¹H-¹³C correlation experiments can be performed with and without ³¹P decoupling during 13C evolution. The intensity ratio of the ¹H 4′/¹³C4′ cross peak from each experiment is proportional to the ³¹P-¹³C4′ J coupling constant.

Correlation of a conformation-dependent property with a bound conformation of a ligand can be achieved by any method that has sufficient sensitivity to detect changes that correlate with changes in bound conformation of a ligand. Such a correlation can be determined by measuring a conformation-dependent property for various conformations of a ligand and determining the extent of change in the signal with change in the conformation. Signal changes that correlate with changes in conformation and that are detectable with a signal to noise ratio accepted in the art as significant can be used in the invention.

Correlation between a conformation-dependent property and a conformation can be determined for a ligand bound to any partner so long as binding is specific and stable. For example, for purposes of establishing a correlation, changes in a conformation dependent property that correlate with changes in bound conformation of a ligand can be determined for a ligand bound to polypeptides from different polypeptide pharmacofamilies. A bound conformation of the ligand in each complex can be determined and a conformation-dependent property can be measured for each complex. Comparison of bound conformations of the ligand in each complex with a measured conformation-dependent property can be used to establish a correlation. Demonstration of a method for establishing a correlation between an NMR signal and bound conformations of a ligand is described herein (see Example IV). Other methods for correlating spectroscopic signals with bound conformations of a ligand are known in the art including, for example, correlation of transferred NOE signals with anti and syn conformations of the nicotinamide ring in NADPH as described in Sem and Kasper Biochemistry 31:3391-3398 (1992). Correlation of transferred NOE signals with conformation is also described in Clore and Gronenborn, J. Magn. Reson. 48:402-417 (1982).

A correlation between a bound conformation and a conformation-dependent property can also be established for a ligand bound to a non-polypeptide binding partner because a conformation-dependent property of a ligand can be independent of interactions that differ between binding partners so long as the ligand is in the same bound conformation when bound to the binding partners. Other binding partners include, for example, nucleic acids, carbohydrates, and synthetic organometallic complexes.

A method of the invention for identifying a conformation-dependent property of a ligand can also include the steps of (a) determining a bound conformation of a ligand, or portion thereof, bound to two or more polypeptides; (b) identifying two or more bound conformations of the ligand, or portion thereof, having substantially the same bound conformation, and (c) identifying a conformation-dependent property of the bound conformations of the ligand, or portion thereof, having substantially the same bound conformation, the conformation-dependent property being correlated with the bound conformation of the ligand, or portion thereof. A conformation-dependent property of a portion of a ligand can be identified, for example, by using the methods described above for identifying a conformation-dependent property of a ligand.

The invention also provides a method for identifying a polypeptide pharmacofamily. The method includes the steps of (a) determining bound conformations of a ligand bound to different polypeptides of a polypeptide family, and (b) identifying two or more bound conformations of the ligand having substantially different bound conformations, thereby identifying at least two polypeptide pharmacofamilies exhibiting binding specificity for the two or more substantially different bound conformations of the ligand.

A method for identifying a polypeptide pharmacofamily can include the steps of (a) determining bound conformations of a ligand bound to different polypeptides of a polypeptide family; (b) clustering bound conformations of a ligand having substantially the same conformations into pharmacoclusters; and (c) identifying a first polypeptide that binds a bound conformation of a ligand in one pharmacocluster and a second polypeptide that binds a bound conformation of a ligand in a second pharmacocluster as belonging to separate polypeptide pharmacofamilies.

Polypeptides of a polypeptide family can be identified by their ability to specifically bind to the same ligand, or portion thereof. Specific binding between a polypeptide and a ligand can be identified by methods known in the art. Methods of determining specific binding include, for example, equilibrium binding analysis, competition assays, and kinetic assays as described in Segel, Enzyme Kinetics John Wiley and Sons, New York (1975), and Kyte, Mechanism in Protein Chemistry Garland Pub. (1995). Thermodynamic and kinetic constants can be used to identify and compare polypeptides and ligands that specifically bind each other and include, for example, dissociation constant (K_{d}), association constant (Kₐ), Michaelis constant (Kₘ), inhibitor dissociation constant (Kᵢₛ) association rate constant (kₒₙ) or dissociation rate constant (k_{off}). For example, a family can be identified as having members that can specifically bind a ligand with a K_{d} of at most 10⁻³ M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M or lower.

A family of polypeptides that bind a ligand can contain a pharmacofamily that binds substantially the same conformation of the ligand, or portion thereof. The methods can be used to identify any number of pharmacofamilies in a family according to the number of different bound conformations of a ligand identified. In cases where two or more polypeptide pharmacofamilies reside in a polypeptide family, the pharmacofamilies can be distinguished according to differences in bound conformations of a ligand bound to the polypeptides. In this case, a bound conformation of a ligand can be determined and compared according to the methods described herein. Polypeptides bound to different bound conformations of a ligand can be identified as those that do not show substantial overlap of all corresponding atoms when bound conformations are overlaid. Thus, polypeptides that bind different bound conformations of a ligand can be separated into different pharmacofamilies. Pharmacofamilies in turn can be identified as containing polypeptides that bind substantially the same bound conformation of a ligand (see Examples II and III).

A pharmacofamily of polypeptides identified by the methods of the invention can have additional similarities that correlate with similarities in bound conformation of a ligand. For example, a polypeptide pharmacofamily identified by the methods of the invention can consist of polypeptide members that share characteristics that are unique to the pharmacofamily when compared to one or more other polypeptides in a different pharmacofamily of the same family. Such characteristics can include, for example, protein fold, evolutionary relatedness, enzymatic activity, domain structure, subcellular localization, interaction partners, or participation in a similar metabolic or signal transduction pathway. A demonstration of a correlation between ligand bound conformation and another characteristic of polypeptides in a pharmacofamily is provided in Example II, which describes correlation of bound conformation of a ligand with polypeptide structure.

An example of a polypeptide family having multiple pharmacofamilies that can be identified by the methods of the invention includes NAD(P)(H) binding polypeptides. Polypeptide pharmacofamilies identified according to differences in bound conformations of NAD(P)(H) are described in Example II and Table 11. Thus, the methods can be used to identify a polypeptide pharmacofamily selected from the group consisting of pharmacofamily 1, pharmacofamily 2, pharmacofamily 3, pharmacofamily 4, pharmacofamily 5, pharmacofamily 6, pharmacofamily 7, and pharmacofamily 8.

The invention provides a polypeptide pharmacofamily, comprising polypeptides that bind to substantially the same bound conformation of a nicotinamide adenine dinucleotide-related molecule selected from pharmacofamily 1, pharmacofamily 2, pharmacofamily 3, pharmacofamily 4, pharmacofamily 5, pharmacofamily 6, pharmacofamily 7, and pharmacofamily 8 as listed in Table 11.

Pharmacofamilies 1 through 8 consist of the polypeptide members provided in Table 11 (see Example II). The polypeptides in pharmacofamily 1 have the NAD(P)(H) binding Rossman fold in common, are all in the NAD(P)(H) binding Rossman SCOP Superfamily, and fall into the SCOP families of the amino-terminal domain of glyceraldehyde-3-phosphate dehydrogenase, the carboxy-terminal domain of alcohol/glucose dehydrogenase, the NAD binding domain of formate/glycerate dehydrogenase, the carboxy-terminal domain of amino acid dehydrogenase, or the amino-terminal domain of lactate & malate dehydrogenase.

The polypeptides in pharmacofamily 2 have the NAD(P) (H) binding Rossman fold in common, are all in the NAD(P) (H) binding Rossman SCOP Superfamily, and fall into the SCOP families of the carboxy-terminal domain of amino acid dehydrogenase, glyceraldehyde-3-phosphate dehydrogenase, and 6-phosphogluconate dehydrogenase.

The polypeptides in pharmacofamily 3 have the NAD(P) (H) binding Rossman fold in common, are all in the NAD(P) (H) binding Rossman SCOP Superfamily, and fall into the tyrosine-dependent oxidoreductase SCOP family.

The polypeptides in pharmacofamily 4 have the heme-linked catalase fold and are in the heme-linked catalase SCOP superfamily and heme-linked catalase SCOP family.

The polypeptides in pharmacofamily 5 have the β-α TIM barrel fold in common, are all in the NAD(P) (H) linked oxidoreductase SCOP Superfamily, and fall into the aldo-keto reductase SCOP family.

The polypeptides in pharmacofamily 6 are dihydrofolate reductases that all show the dihydrofolate reductase fold and fall into the dihydrofolate reductase SCOP superfamily and family.

The polypeptides in pharmacofamily 7 have the FAD/NAD(P) (H) binding domain fold in common, are all in the FAD/NAD(P) (H) binding domain SCOP Superfamily, and fall into the the amino-terminal and central domains of FAD/NAD linked reductase SCOP family.

The polypeptides in pharmacofamily 8 have the ferrodoxin like fold in common, are all in the ferrodoxin like SCOP Superfamily, and fall into the NADPH-cytochrome P450 reductase or reductase SCOP families.

Polypeptide pharmacofamilies 1 through 8 were identified according to binding interactions with bound conformations of NAD(P) (H) in pharmacoclusters 1 through 8, as described in Example II. Accordingly, the invention provides a polypeptide pharmacofamily, comprising polypeptides that bind to a nicotinamide adenine dinucleotide-related molecule having a bound conformation selected from pharmacocluster 1, pharmacocluster 2, pharmacocluster 3, pharmacocluster 4, pharmacocluster 5, pharmacocluster 6, pharmacocluster 7, and pharmacocluster 8.

The invention additionally provides a method for identifying a member of a polypeptide pharmacofamily. The method consists of (a) determining a conformation-dependent property of a ligand bound to a polypeptide, and (b) determining a pharmacocluster having substantially the same conformation-dependent property as the conformation-dependent property determined for the bound ligand, wherein a polypeptide pharmacofamily binds the ligand in a conformation of the pharmacocluster, thereby identifying the polypeptide as a member of the polypeptide pharmacofamily. For example, the method can be used with a ligand such as a nicotinamide adenine dinucleotide-related molecule or adenosine phosphate-related molecule (see Examples II and III).

The methods of the invention allow a new member of a polypeptide pharmacofamily to be identified based on correlation of a conformation-dependent property of a bound conformation of a ligand bound to a polypeptide with a conformation-dependent property established for a bound conformation of the ligand bound to another polypeptide in the same pharmacofamily. Thus, a classification can be made based on ligand structure without requiring determination of the bound conformation of the ligand. In one embodiment, the conformation-dependent property can be a model of a bound conformation. A bound conformation of a ligand bound to a test polypeptide can be determined, and the bound conformation can be compared to a pharmacocluster according to the methods described herein. Substantial overlap between the bound conformation of the ligand bound to the test polypeptide and another bound conformation of the ligand bound to a polypeptide in a pharmacofamily can be used to identify the test polypeptide as a member of that polypeptide pharmacofamily.

In another embodiment, the conformation-dependent property can be a spectroscopic signal that is correlated with the conformation of a ligand. A spectroscopic signal can be measured for the ligand bound to a test polypeptide. The signal can be compared to a signal correlated with a bound conformation of a ligand bound to a polypeptide in a polypeptide pharmacofamily. Substantial similarity between the two signals indicates that the bound conformation of the ligand bound to the test polypeptide is substantially similar to the bound conformation of the ligand bound to the polypeptides of the pharmacofamily. Thus, the test polypeptide can be identified as a member of the polypeptide pharmacofamily.

The invention provides rapid and efficient methods that can be used in a high-throughput screening format. High-throughput methods can be useful for identifying a member of a polypeptide pharmacofamily. In a case where a conformation-dependent property can be rapidly detected and processed, automated methods can be created for measuring samples in rapid succession or measuring multiple samples in parallel. Automated methods can be used for rapidly handling samples including, for example, robotic instruments. A combination of automated sample handling methods with detection of a conformation-dependent property can, therefore, be useful in a high-throughput screening method.

According to the methods of the invention a compound can be identified that has greater specificity for the polypeptides of one pharmacofamily than for other polypeptides in the same family. Such a compound can be used to identify new members of a pharmacophore family using a binding assay. For example, a mimetic or analog of a ligand can be identified that preferentially adopts a conformation more similar to conformations in a particular pharmacocluster than those in other pharmacoclusters. Such a mimetic or analog can be used in a any binding assay capable of detecting interactions with a polypeptide, including, for example, high-throughput methods.

A member of a polypeptide pharmacofamily can also be identified by searching a database of bound conformations of a ligand. For example, a bound conformation of a ligand that binds to a polypeptide of an identified pharmacofamily can be used as a query in a 3 dimensional search of a database containing bound conformations of a ligand. Overlap between the query conformation and a retrieved bound conformation of the ligand can be used to identify a polypeptide bound to the retrieved bound conformation of the ligand as a member of the same polypeptide pharmacofamily as a polypeptide that binds the query bound conformation (see Example I).

The invention also provides a method of modeling the three dimensional structure of a polypeptide. The method consists of (a) determining a conformation-dependent property of a ligand bound to a polypeptide; (b) determining a pharmacocluster having substantially the same conformation-dependent property as the conformation-dependent property determined for the bound ligand, wherein a polypeptide pharmacofamily binds the ligand in a conformation of the pharmacocluster, thereby identifying the polypeptide as a member of the polypeptide pharmacofamily, and (c) modeling the three dimensional structure of the polypeptide according to a structural model of the second member of the polypeptide pharmacofamily.

As disclosed herein, polypeptides in a pharmacofamily can have similar characteristics including, for example, similar 3 dimensional structure. Therefore, the 3 dimensional structure of a polypeptide identified by the invention as a member of a pharmacofamily can be modeled using a polypeptide that is in the same pharmacofamily and for which the structure is known. A variety of methods are known in the art for modeling the three dimensional structure of a polypeptide according to the amino acid sequence of the polypeptide and a structure of a second polypeptide used as a template. Available algorithms include, for example, GRASP (Nicholls, A., supra), ALADDIN (Van Drie et al. supra), INSIGHT98 (Molecular Simulations Inc., San Diego Calif.), RASMOL (Sayle et al., Trends Biochem Sci. 20:374-376 (1995)) and MOLMOL (Koradi et al., J. Mol. Graphics 14:51-55 (1996 )).

A model of a polypeptide determined by the methods of the invention can be useful for identifying a function of the polypeptide. For example, residues of a polypeptide that are involved in binding can be identified using a model of the invention. Residues identified as participating in binding can be modified, for example, to engineer new functions into a polypeptide, to reduce an intrinsic activity of a polypeptide, or to enhance an intrinsic activity of a polypeptide. In another example, a model of a polypeptide can be compared to other polypeptide structures to identify similar functions. Exemplary functions that can be identified from a polypeptide structure include binding interactions with other polypeptides and catalytic activities.

The invention also provides a method for constructing a ligand conformer model by determining an average structure of the bound conformations of a ligand in a pharmacocluster. A method for constructing a ligand conformer model can include the steps of (a) determining bound conformations of a ligand bound to different polypeptides; (b) clustering two or more bound conformations of the ligand having substantially the same bound conformation, thereby identifying a pharmacocluster, and (c) determining an average structure of the bound conformations of the ligand in the pharmacocluster. Additionally, a method for constructing a ligand conformer model can include the steps of (a) determining a bound conformation of a ligand bound to a polypeptide; (b) determining a pharmacocluster having substantially the same bound conformation as the bound conformation, thereby identifying the bound conformation of the ligand as a member of the pharmacocluster, and (c) determining an average structure of the bound conformations of the ligand in the pharmacocluster.

An average structure of the bound conformations of a ligand in a pharmacocluster can be determined by a variety of methods known in the art. For example, an average structure can be determined by overlaying bound conformations, or portions thereof, and identifying an average location for each atom. Bound conformations in a group to be averaged can be overlayed relative to a single member or relative to a centroid position for each atom. Algorithms for determining an average structure are known in the art and include for example the OVERLAY routine in INSIGHT98 (Molecular Simulations Inc., San Diego Calif.).

The format of a ligand conformer model can be chosen based on the method used to generate the model and the desired use of the model. In this regard, a conformer model can be represented as a single structure. The resulting structure can be a unique structure compared to the conformations in the pharmacocluster from which it was derived. Thus, the conformer model can be a new structure never before observed in nature. A model represented by a single structure can be useful for making visual comparisons by overlaying other structures with the model. A conformer model can also be represented as a plurality of structures incorporating all or a subset of the bound conformations in the pharmacocluster. A model represented by multiple structures can be useful for identifying a range of minor deviations in the model.

In yet another representation, the conformer model can be a volume surrounding all or a subset of the bound conformations in the pharmacocluster. A model showing volume can be useful for comparing other structures in a fitting format such that a structure which fits within the volume of the model can be identified as substantially similar to the model. One approach that can be used to fit a structure to a volume is comparison of equivalent surface patches using gnomonic projection as described for example in Chau and Dean, J. Mol. Graphics 7:130 (1989). Use of a gnomonic projection to compare structures is also described in Doucet and Weber, Computer-Aided Molecular Design: Theory and Applications, Academic Press, San Diego Calif. (1996). Algorithms which can be used to fit a structure to a volume are known in the art and include, for example, CATALYST (Molecular Simulations Inc., San Diego, CA) and THREEDOM which is a part of the INTERCHEM package which makes use of an Icosahedral Matching Algorithm (Bladon, J. Mol. Graphics 7:130 (1989) for the comparison and alignment of structures. An exemplary method of identifying a binding compound by searching a database of structures using a gnomonic projection is provided in Example V.

A conformer model can be useful in querying a database of polypeptide structures to find other members of a polypeptide pharmacofamily. For example, a member of a polypeptide pharmacofamily can be identified by querying a database of bound conformations of a ligand to identify a retrieved bound conformation of a ligand that is substantially similar to the query structure, thereby identifying a polypeptide bound to the retrieved bound conformation as a member of the same pharmacofamily as a polypeptide bound to the query bound conformation. A conformer model can also be used to identify a new member of a polypeptide pharmacofamily by querying a database of one or more polypeptide structures using an algorithm that docks the conformer model, wherein a favorable docking result with a retrieved polypeptide indicates that the retrieved polypeptide is a member of the same polypeptide pharmacofamily as a polypeptide bound to the bound conformation used as a query. In the latter mode, a potential new member of a pharmacofamily from which the conformer model was derived can be identified. The database queries described above can be performed with algorithms available in the art including, for example, THREEDOM and CATALYST.

An advantage of the invention is that a conformer model can be used to identify a binding compound that is specific for polypeptides of a pharmacofamily. For example, the conformer model can be compared to a structure of a compound or to a bound conformation of a ligand to identify those having similar conformation. A conformer model can be further used to query a database of compounds to identify individual compounds having similar conformations.

A conformer model of the invention can also be used to design a binding compound that is specific for polypeptides of one or more pharmacofamilies. The methods of the invention provide a conformer model that can be produced according to a cluster of bound conformations of a ligand that are specific for polypeptides of a pharmacofamily. A conformer model identified by these criteria can be used as a scaffold structure for developing a compound having enhanced binding affinity or specificity for polypeptides of a pharmacofamily. Such a scaffold can also be used to design a combinatorial synthesis producing a library of compounds which can be screened for enhanced binding affinity for polypeptide members of a pharmacofamily or specificity for polypeptide members of one pharmacofamily compared to polypeptide members of another pharmacofamily. An algorithm can be used to design a binding compound based on a conformer model including, for example, LUDI as described by Bohm, J. Comput. Aided Mol. Des. 6:61-78 (1992).

A conformer model need not include all atoms of a pharmacocluster. Thus, a conformer model can include a portion of atoms in a pharmacocluster so long as the portion consists of contiguous atoms of a bound conformation of a ligand and provides sufficient information to distinguish one pharmacocluster from another. Thus, a conformer model can be constructed by overlaying corresponding fragments of bound conformations of a ligand and obtaining an average structure according to the methods described above. A conformer model made from a portion of a ligand can be advantageous due to its small size compared to a complete structure of the ligand from which it was derived. A conformer model based on a portion of a bound conformation of a ligand can also be used to more efficiently and rapidly query a database due to a reduced use of computer memory compared to the memory required to manipulate and store a structure containing all atoms of the ligand.

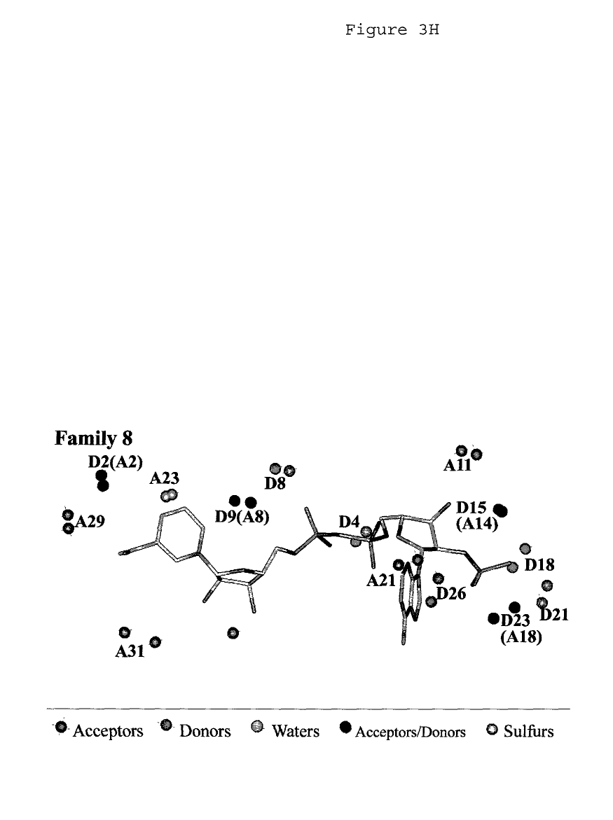

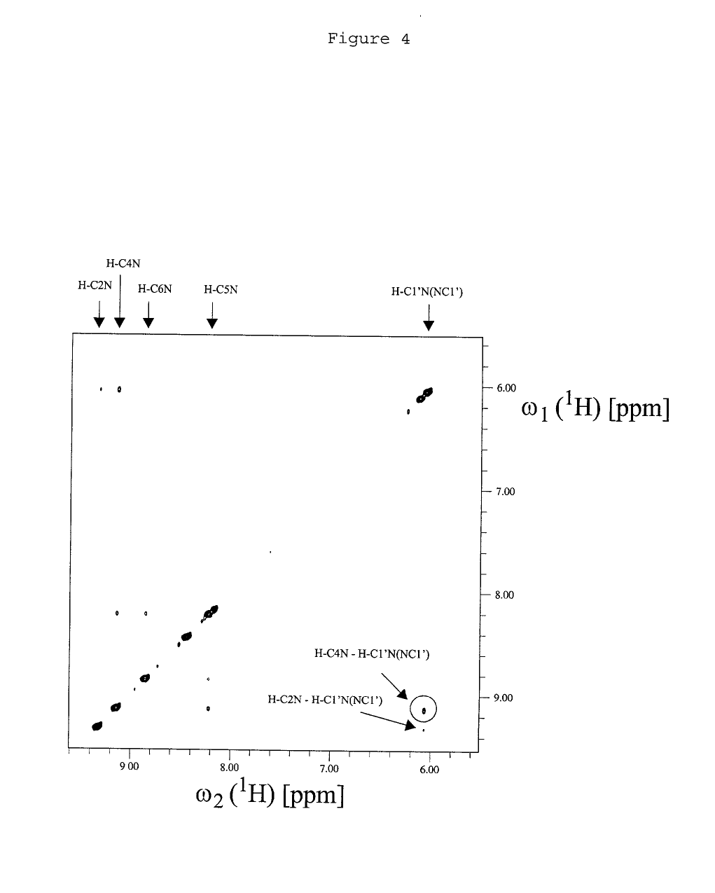

The invention provides a ligand conformer model, selected from the group consisting of conformer model 1 having coordinates listed in Table 3C, conformer model 2 having coordinates listed in Table 4C, conformer model 3 having coordinates listed in Table 5C, conformer model 4 having coordinates listed in Table 6C, conformer model 5 having coordinates listed in Table 7C, conformer model 6 having coordinates listed in Table 8C, conformer model 7 having coordinates listed in Table 9C, and conformer model 8 having coordinates listed in Table 10C. Conformer models 1-8 are average structures calculated from pharmacoclusters 1-8 respectively. The conformer models were determined as described in Example III and are shown in FIG. 4.

The invention also provides moiety, having coordinates listed in Table 3C, coordinates listed in Table 4C, coordinates listed in Table 5C, coordinates listed in Table 6C, coordinates listed in Table 7C, coordinates listed in Table 8C, coordinates listed in Table 9C, or coordinates listed in Table 10C or subsets of the respective coordinate sets thereof. In one embodiment the moiety is not nicotinamide adenine dinucleotide or nicotinamide adenine dinucleotide phosphate.

Additionally, the invention provides a method for constructing a pharmacophore model by constructing a model that contains one or more selected conformation-dependent properties of one or more pharmacoclusters. A method for constructing a pharmacophore model can include the steps of (a) determining bound conformations of a ligand bound to different polypeptides; (b) identifying two or more bound conformations of the ligand having substantially the same bound conformation; (c) identifying a conformation-dependent property of the bound conformations of the ligand having substantially the same bound conformation, the conformation-dependent property being correlated with the bound conformation of the ligand, and (d) constructing a model that contains one or more selected conformation-dependent properties of one or more pharmacoclusters.

Additionally, a method for constructing a pharmacophore model can include the steps of (a) determining bound conformations of a ligand, or portion thereof, bound to different polypeptides; (b) clustering two or more bound conformations of the ligand, or portion thereof, having substantially the same bound conformation, thereby identifying a pharmacocluster, and (c) determining an average structure of the bound conformations of the ligand, or portion thereof, in the pharmacocluster, wherein the average structure is a pharmacophore model. A method for constructing a ligand conformer model can also include the steps of (a) determining a bound conformation of a ligand, or portion thereof, bound to a polypeptide; (b) determining a pharmacocluster having substantially the same bound conformation as the bound conformation, thereby identifying the bound conformation of the ligand as a member of the pharmacocluster, and (c) determining an average structure of the bound conformations of the ligand in the pharmacocluster, wherein the average structure is a pharmacophore model.

A pharmacophore model constructed by the methods of the invention can be derived from any conformation-dependent property that is correlated with a pharmacocluster. An example of a pharmacophore model useful in the methods of the invention is a conformer model. Additionally, a pharmacophore model can include a portion of a bound conformation, wherein the portion need not contain contiguous atoms of a bound conformation of a ligand so long as the pharmacophore model provides sufficient information to distinguish one pharmacocluster from another. Thus, a pharmacophore model can appear as points in space unconnected by any semblance of a covalent bond due to absence of intervening atoms. For example, a pharmacophore model constructed from a pharmacocluster of nicotinamide adenine dinucleotide bound conformations can contain a phosphate moiety and nicotinamide ring moiety absent the ribose moiety which intervenes in a complete model of the structure.

A pharmacophore model can be any representation of points in a defined coordinate system that correspond to positions of atoms in a bound conformation of a ligand. For example, a point in a pharmacophore model can correlate with the center of an atom in a conformer model. An atom of a conformer model can also be represented by a series of points forming a line, plane or sphere. A line, plane or sphere can form a geometric representation designating, for example, shape of one or more atoms or volume occupied by one or more atoms.

A pharmacophore model can be represented in any coordinate system including, for example, a 2 dimensional Cartesian coordinate system or 3 dimensional Cartesian coordinate system. Other coordinate systems that can be used include a fractional coordinate system or reciprocal space such as those used in crystallographic calculations which are described in Stout and Jensen, supra.

In addition to a geometric description of a bound conformation of a ligand, a pharmacophore model can include other characteristics of atoms or moieties of the ligand including, for example, charge or hydrophobicity. Thus, a pharmacophore model can be a generalized structure, which includes but does not unambiguously describe the bound conformations of the ligand bound to the polypeptides in the pharmacofamily from which it was derived. For example, atoms can be represented as units of charge such that an oxygen in a bound conformation of a ligand can be represented by an electronegative point in the pharmacophore model. In this example, the electronegative point in the pharmacophore model includes any electronegative atom at that particular location including, for example, an oxygen or sulfur.

A pharmacophore model can be constructed to include, in addition to characteristics of the ligand itself, characteristics of an atom or moiety that interacts with the ligand and from a bound polypeptide. Characteristics of an interacting polypeptide atom or moiety that can be included in a pharmacophore model include, for example, atomic number, volume occupied, distance from an atom of the ligand, charge, hydrophobicity, polarity, or location relative to the ligand. Methods for constructing a pharmacophore model to include interacting atoms from a polypeptide are provided in Example III.

A characteristic included in a pharmacophore model can be incorporated into a geometric representation using any additional representation that can be correlated with the characteristic. For example, use of color or shading can be used to identify regions having characteristics such as charge, polarity, or hydrophobicity. As such, the depth of shading or color or the hue of color can be used to determine the degree of a characteristic. By way of example, a common convention used in the art is to identify regions of increased positive charge with deeper shades of blue, areas of increased negative charge with deeper shades of red and neutral regions with white. Numeric representations can also be used in a pharmacophore model including, for example, values corresponding to potential energy for an interaction, or degree of polarity.

In addition, a pharmacophore model can incorporate constraints of a physical or chemical property of the bound conformations of a ligand in a pharmacocluster. A constraint of a physical property can be, for example, a distance between two atoms, allowed torsion angle of a bond, or volume of space occupied by an atom or moiety. A constraint of a chemical property can be, for example, polarity, van der Waals interaction, hydrogen bond, ionic bond, or hydrophobic interaction. Such constraints can be included in a pharmacophore model using the representations described above.

A pharmacophore model can include two or more pharmacoclusters. In order to identify a ligand having broad specificity for two or more polypeptide pharmacofamilies, a pharmacophore model can be derived from the two or more corresponding pharmacoclusters. Additionally, in order to identify a ligand that can preferentially bind a first polypeptide which belongs to a first polypeptide pharmacofamily compared to a second polypeptide of a second polypeptide pharmacofamily, a pharmacophore model can incorporate constraints on geometry or any other characteristic so as to exclude a characteristic of the bound conformation of the ligand bound to the second polypeptide. For example, a geometric constraint can be a forbidden region for one or more atom of a bound conformation of a ligand. A forbidden region can be identified by overlaying two conformer models in a coordinate system and identifying a coordinate or set of coordinates differentially occupied by one or more atoms of the conformer models. A pharmacophore model incorporating a forbidden region as such will be specific for a polypeptide of one pharmacofamily over a polypeptide of a second pharmacofamily correspondent with the constraint incorporated.

An advantage of the invention is that a pharmacophore model can be created based on multiple structures of the same ligand. In comparison to a pharmacophore model derived from a single structure or different ligands, a pharmacophore model derived from multiple bound conformations of the same ligand can include a greater degree of geometric information. For example, averaging of multiple bound conformations of the same ligand can provide torsion angle constraints that are not available from a single structure and not evident from comparing different ligands.

The invention further provides a method for identifying a binding compound for one or more members of a polypeptide pharmacofamily by identifying a compound having a selected conformation-dependent property of a pharmacocluster. A binding compound can be any molecule having selected conformation-dependent properties of a ligand such that the binding compound can form a complex with one or more members of one or more polypeptide pharmacofamily. A method for identifying a binding compound for one or more members of a polypeptide pharmacofamily can include the steps of contacting a ligand with a polypeptide member of a pharmacofamily; identifying a conformation-dependent property associated with a bound conformation of the ligand bound to the polypeptide; comparing the conformation-dependent property of the bound conformation of the ligand bound to the polypeptide with a conformation-dependent property of a bound conformation of a ligand bound to another polypeptide in the same pharmacofamily; and identifying a ligand bound to the polypeptide with a conformation-dependent property similar to a bound conformation of a ligand bound to another polypeptide in the same pharmacofamily, thereby identifying a compound that binds one or more polypeptide members of a pharmacofamily. A compound that binds to one or more members of a polypeptide pharmacofamily can be identified by determining a conformation-dependent property by any of the methods described herein. For example, a ligand conformation or spectroscopic signal can provide a conformation-dependent property useful in identifying a compound that binds to one or more members of a polypeptide pharmacofamily.

The methods described herein for identifying a binding compound for one or more members of a polypeptide pharmacofamily can readily be adapted to a high throughput screening method. For example, methods of rapidly detecting a conformation-dependent property in a sequence of samples or detecting a conformation-dependent property in parallel samples can be applied to a high-throughput screen. One skilled in the art will know how to adapt the methods described here to a high throughput screening format using, for example, robotic manipulation of samples.

A method for identifying a binding compound for one or more members of a polypeptide pharmacofamily can include the steps of determining a bound conformation of a ligand bound to a polypeptide member of a polypeptide pharmacofamily; comparing the bound conformation of the ligand bound to the polypeptide member of the polypeptide pharmacofamily to a pharmacophore model; and identifying the bound conformation of the ligand bound to the polypeptide member of the polypeptide pharmacofamily that satisfies the constraints of the pharmacophore model as a binding compound for one or more members of the pharmacofamily in which the polypeptide member belongs.

A pharmacophore model can be useful in querying a database of polypeptide structures to find other members of a polypeptide pharmacofamily. For example, a member of a polypeptide pharmacofamily can be identified by querying a database of bound conformations of a ligand to retrieve a structure that fits the constraints of the query pharmacophore model, thereby identifying the retrieved polypeptide as a member of the pharmacofamily from which the pharmacophore model was derived. A pharmacophore model can also be used to identify a new member of a polypeptide pharmacofamily by querying a database of one or more polypeptide structures using an algorithm that docks or compares the pharmacophore model to polypeptide structures, wherein a favorable docking or comparison identifies a polypeptide as a member of the same polypeptide pharmacofamily from which the pharmacophore model was derived. The database queries described above can be performed with algorithms available in the art including, for example, THREEDOM and CATALYST.

An advantage of the invention is that a pharmacophore model can also be used to identify a binding compound that is specific for polypeptides of one or more pharmacofamilies. For example, a pharmacophore model can be compared to a structure of a compound or to a bound conformation of a ligand to identify those having similar properties. A conformer model can be further used to query a database of compounds to identify individual compounds having similar properties.

A pharmacophore model of the invention can also be used to design a binding compound that is specific for polypeptides of one or more pharmacofamilies. A pharmacophore model identified by these criteria can be used as a scaffold or set of constraints for developing a compound having enhanced binding affinity or specificity for polypeptides of of one or more pharmacofamilies. Using similar methods a pharmacophore model can be used to design a combinatorial synthesis producing a library of compounds having properties consistent or similar to the model which can be then be screened for enhanced binding affinity or specificity for polypeptide members of one or more pharmacofamilies. An algorithm can be used to design a binding compound based on a pharmacophore model including, for example, LUDI as described by Bohm, J. Comput. Aided Mol. Des. 6:61-78 (1992).

A compound can be identified as satisfying the constraints of a pharmacophore model by a variety of methods for comparing structures. For example, a pharmacophore model that is a geometric representation such as a conformer model can be overlaid with a compound, and the best fit determined as described herein. Substantial overlap between a compound and a pharmacophore model can be indicated by a visual comparison and/or computation based comparison based on for example, RMSD values or torsion angle values as described above. In a case where a pharmacophore model is represented by constraints, a compound can be fitted to the pharmacophore model to identify if the properties of the compound satisfy the constraints of the pharmacophore model. For example, if a pharmacophore model contains, as a constraint, a maximum distance between atoms, a compound that satisfies the constraint can be identified as having a bond distance between corresponding atoms that is at least the maximum value. One skilled in the art will know how to extend such methods of comparison to any physical or chemical constraint.

A compound can also be identified as satisfying the constraints of a pharmacophore model by demonstrating the same characteristics for one or more specific atom located within a volume of space defined by the geometric constraints of the pharmacophore model. For example, in a case where polarity is a constraint and where a conformation of a compound can be overlaid with a pharmacophore model, an atom that overlaps a volume of space indicated by the pharmacophore and having polarity within the defined limits can be identified as satisfying constraints of the pharmacophore. By extension, a compound having atoms which satisfy all constraints of a pharmacophore is identified as a binding compound for one or more members of a polypeptide pharmacofamily from which the pharmacophore was produced.

Therefore, the invention provides a binding compound identified by the above described methods. For example, the invention provides a binding compound identified using a pharmacophore model or a conformer model derived from a pharmacocluster and/or pharmacofamily.

The invention provides a pharmacophore model, selected from the group consisting of pharmacophore model 1 having coordinates listed in Tables 3B and 3C, pharmacophore model 2 having coordinates listed in Tables 4B and 4C, pharmacophore model 3 having coordinates listed in Tables 5B and 5C, pharmacophore model 4 having coordinates listed in Tables 6B and 6C, pharmacophore model 5 having coordinates listed in Tables 7B and 7C, pharmacophore model 6 having coordinates listed in Tables 8B and 8C, pharmacophore model 7 having coordinates listed in Tables 9B and 9C, and pharmacophore model 8 having coordinates listed in Tables 10B and 10C.

The invention also provides a medium comprising a storage medium and stored in the medium, atom coordinates selected from the atomic coordinates listed in Table 3B, 3C, 4B, 4C, 5B, 5C, 6B, 6C, 7B, 7C, 8B, 8C, 9B, 9C, 10B or 10C, or a subset thereof. In one embodiment the medium comprises a computer readable medium. The use of a computer apparatus is convenient since atomic coordinates can be conveniently stored and accessed for manipulation including, for example, docking to a polypeptide structure or comparison to coordinates for other bound conformations of a ligand. Exemplary methods for manipulating atomic coordinates are described above.

It is understood that a computer apparatus of the invention need not itself store atomic coordinates of the invention. The computer apparatus contains an algorithm for viewing a structure from the coordinates or otherwise manipulating the coordinates. By using various hardware, software and network combinations, the atomic coordinates can be manipulated in a variety of configurations. Such a separate medium can be another computer apparatus, a storage medium such as a floppy disk, Zip disk or a server such as a file-server, which can be accessed by a carrier wave such as an electromagnetic carrier wave. One skilled in the art will know or can readily determine appropriate hardware, software or network interfaces that allow interconnection of an invention computer apparatus.

The methods of the invention described herein can be performed in a computer apparatus using the atomic coordinates listed in Table 3B, 3C, 4B, 4C, 5B, 5C, 6B, 6C, 7B, 7C, 8B, 8C, 9B, 9C, 10B or 10C by adding the step of entering the coordinates or a subset of the coordinates to the computer apparatus that performs a method of the invention. One skilled in the art will know or can readily determine an algorithm instructing a computer apparatus to carry out the methods of the invention.

It is understood that modifications which do not substantially affect the activity of the various embodiments of this invention are also provided within the definition of the invention provided herein. Accordingly, the following examples are intended to illustrate but not limit the present invention.

### EXAMPLE I

Identification of Polypeptide Pharmacofamilies Based on Bound Conformations of NAD(P)(H) Ligands

This example describes identification of ligand conformer groups and corresponding polypeptide pharmacofamilies based on bound conformations of NAD(P)(H) bound to polypeptide oxidoreductases.

The oxidoreductases form a family of polypeptides that bind NAD(H) and NADP(H). In order to identify pharmacofamilies within the family of oxidoreductases, bound conformations of NAD(P)(H) were determined by searching the protein databank. Bound conformations from 156 structures were clustered into separate pharmacoclusters, and pharmacofamilies were identified according to binding to bound conformations of NAD(P)(H) in separate pharmacoclusters.

Structure files containing polypeptides with bound NAD(P)(H) were identified from the protein databank by keyword searches using the database software. Keywords included “NAD,” “NADH,” “NADP,” “NADPH,” “oxidoreductase,” “dehydrogenase” and “reductase.” Cluster analysis was performed using the algorithm COMPARE (Chiron Corp, 1995; distributed by Quantum Chemistry program Exchange, Indianapolis Ind.) in combination with visual inspection. All clusters were visually inspected using Insight 98 for outliers that demonstrated poor overlay with the rest of the pharmacocluster as a whole. These outliers were compared against each other and existing pharmacoclusters to find other possible matches. Those that did not fit any family were removed. Comparison between bound conformations was made based on the RMSD equations supplied in COMPARE.

Eight pharmacoclusters were identified by this method, as shown in FIG. 1. Visual inspection of the clusters in FIG. 1 demonstrates that members within a cluster are substantially overlapped. Comparison between clusters demonstrates substantial differences. For example, the bound conformations in cluster 5 have an extended structure compared to the bound conformations in cluster 4, which form a horseshoe like shape. Other differences include, for example, a flip in the nicotinamide ring between cluster 1 and cluster 2 such that the nicotinamide ring is anti to the ribose in cluster 1 and syn to the ribose in cluster 2 and a change in torsion angle in the bonds connecting the adenine ribose to the adenine phosphate for the bound conformations of cluster 3 compared to those of cluster 2.

The dihedral angles for various bonds in the bound conformations of the NADP(H) ligand can be used to distinguish the pharmacoclusters. As shown in Table 1 (see FIG. 2 for atom and bond locations), although many dihedral angles are similar between two or more pharmacoclusters, each pharmacocluster can be distinguished from the others by comparison of the full set of dihedral angles. For example, pharmacoclusters 2 and 3 can be distinguished by comparison between the dihedral angles at O4′A-C4′A-C540 A-O5′A which are 154 degrees and −131 degrees respectively and by comparison between the dihedral angles at C5′A-O5′A-PA-O3 which are 105 degrees and 57 degrees respectively.

TABLE 1
Diedral Angles for Pharmacoclusters

| | PC1 | | PC2 | | PC3 | | PC4 | | PC5 | | PC6 | | PC7 | | PC8 | |
|---|---|---|---|---|---|---|---|---|---|---|---|---|---|---|---|---|
| Dihedral angle | Avg. | std | Avg. | std | Avg. | std | Avg. | std | Avg. | std | Avg. | std | Avg. | std | Avg. | std |
| O4′A-C1′A-N9A-C8A | 75 | 24 | 75 | 11 | 69 | 18 | 85 | 7 | 72 | 3 | 18 | 16 | 81 | 12 | 105 | 6 |
| O4′A-C4′A-C5′A-O5′A | 180 | 19 | 154 | 30 | −131 | 99 | −166 | 12 | 65 | 4 | 79 | 11 | 168 | 12 | −84 | 38 |
| C4′A-C5′A-O5′A-PA | 138 | 86 | 137 | 15 | 121 | 93 | −152 | 2 | 180 | 6 | −156 | 9 | 150 | 21 | −171 | 3 |
| C5′A-O5′A-PA-O3 | 65 | 39 | 105 | 44 | 57 | 44 | 55 | 0 | −71 | 6 | −82 | 7 | 58 | 10 | −34 | 10 |
| O5′A-PA-O3-PN | 97 | 61 | 42 | 77 | 74 | 24 | 115 | 20 | 121 | 30 | 139 | 17 | 75 | 12 | −188 | 16 |
| PA-O3-PN-O5′N | −143 | 72 | −165 | 53 | −136 | 29 | −152 | 10 | 50 | 27 | 84 | 15 | 107 | 27 | 128 | 39 |
| O3-PN-O5′N-C5′N | 70 | 44 | 56 | 86 | 101 | 36 | −64 | 22 | −92 | 13 | 64 | 25 | 27 | 45 | 72 | 7 |
| PN-O5′N-C5′N-C4′N | 181 | 14 | 176 | 41 | 162 | 27 | 145 | 7 | −112 | 26 | 139 | 15 | −136 | 13 | 191 | 18 |
| O5′N-C5′N-C4′N-O4′N | −73 | 46 | −58 | 40 | −54 | 26 | −55 | 10 | −60 | 4 | 65 | 10 | −69 | 13 | 183 | 20 |
| O4′N-C1′N-N1N-C2N | −120 | 24 | 69 | 17 | 53 | 11 | 59 | 5 | −132 | 6 | −117 | 10 | −178 | 16 | −122 | 6 |
| C1′A-C2′A-C3′A-C4′A | −25 | 10 | −29 | 5 | −29 | 10 | −37 | 23 | −30 | 8 | 42 | 6 | −1 | 46 | −33 | 3 |
| C1′N-C2′N-C3′N-C4′N | −36 | 44 | −35 | 6 | −28 | 20 | 22 | 9 | 40 | 2 | −39 | 5 | 17 | 38 | −17 | 3 |

A quantitative analysis of the results of clustering bound conformations of NAD(P)(H) is provided in Table 2. Table 2 shows RMSD values calculated from comparisons between each pharmacocluster's average coordinates. Average coordinates were determined from the pharmacocluster subsets listed in Tables 3 through 10 as described below.

TABLE 2
RMSD between each Pharmacocluster's average coordinates

| | 1 | 2 | 3 | 4 | 5 | 6 | 7 | 8 |
|---|---|---|---|---|---|---|---|---|
| 1 | | 1.89 | 2.24 | 3.81 | 2.31 | 2.74 | 2.68 | 1.42 |
| 2 | | | 0.95 | 3.61 | 2.51 | 3.47 | 2.52 | 2.62 |
| 3 | | | | 3.88 | 2.85 | 3.36 | 3.00 | 3.02 |
| 4 | | | | | 5.22 | 4.67 | 4.54 | 3.71 |
| 5 | | | | | | 2.49 | 1.93 | 2.88 |
| 6 | | | | | | | 2.30 | 2.53 |
| 7 | | | | | | | | 3.06 |
| 8 | | | | | | | | |

Tables 3A, 4A, 5A, 6A, 7A, 8A, 9A and 10A show RMSD values for subsets of members of pharmacoclusters 1-8, respectively. The RMSD values for each member were calculated as comparisons to an average structure for the subsets shown in each table respectively. For each pharmacocluster a subset of the possible ligands that belong to each cluster were identified. Each subset was chosen to maximize the diversity of the family and to minimize over-representation of ligand conformations from enzymes that exist multiply in the PDB database. The goal of the subset selection was to fully represent characteristics from oxidoreductases belonging to a range of species and catalyzing a range of different reactions. For example, there exists over ten alcohol dehydrogenases in the PDB database; however, for purposes of this study, only three were chosen from three different species for use in the 3D overlay and the pharmacophore construction. Average coordinates for the above described pharmacocluster subsets were obtained by overlaying ligand structures in MSI InsightII using the overlay function. The three dimensional coordinates for each atom in each ligand were used to calculate an average position and a standard deviation for the pharmacofamily.

Comparison of the RMSD values in part A of Tables 3 through 10 with the RMSD values in Table 2 demonstrate that a member of a pharmacocluster can be identified as having a lower RMSD compared to an average conformation of the members in its pharmacocluster than the RMSD between each family's average coordinates. In some cases it can be beneficial to combine two or more methods of comparison. For example, as described above pharmacoclusters 2 and 3 which have a relatively low RMSD when compared to each other can be distinguished from each other by visual inspection and by comparison of dihedral angles at various bonds.

These results demonstrate that bound conformations of a ligand can be grouped into pharmacoclusters by methods of structure comparison. These results also demonstrate methods for distinguishing pharmacoclusters and members within pharmacoclusters.

### EXAMPLE II

### Correlation Between the Structure of Polypeptides and the Bound Conformations of NAD(P)(H)

This example describes a correlation between bound conformations of NAD(P)(H) and structural classification of polypeptides such that polypeptides of a pharmacofamily have similar protein fold.

Pharmacoclusters for conformations of NAD(P)(H) bound to oxidoreductase polypeptides were clustered as described in Example I. For each polypeptide the protein fold, SCOP super-family designation and SCOP family designation was identified from the SCOP website administered by Laboratory of Molecular Biology at the MRC, Cambridge England (http://mrc-lmb.cam.ac.uk).

Table 11 shows the grouping of NAD(P)(H) binding polypeptides into 8 pharmacofamilies.

TABLE 11
Pharmacofamilies

| | | | | | |
|---|---|---|---|---|---|
| Polypeptide | Source | PDB | Fold | SCOP-Superfamily | SCOP-Family |

Family 1: NAD(P) Rossman Binding Domain (anti)

| | | | | | |
|---|---|---|---|---|---|
| Alcohol Dehydrogenase | Horse | 1a71 | NAD(P) binding | NAD(P) binding | Alcohol/glucose |
| | Liver | | Rossman | Rossman | dehydrog. |
| Alcohol Dehydrogenase | human | 1agn | NAD(P) binding | NAD(P) binding | Alcohol/glucose |
| | | | Rossman | Rossman | dehydrog. |
| Alcohol Dehydrogenase | Human | 1dlt | NAD(P) binding | NAD(P) binding | Alcohol/glucose |
| | | | Rossman | Rossman | dehydrog. |
| Alcohol Dehydrogenase | Horse | 1axe | NAD(P) binding | NAD(P) binding | Alcohol/glucose |
| | Liver | | Rossman | Rossman | dehydrog. |
| Alcohol Dehydrogenase | Horse | 1axg | NAD(P) binding | NAD(P) binding | Alcohol/glucose |
| | Liver | | Rossman | Rossman | dehydrog. |
| Alcohol Dehydrogenase | cod fish | 1cdo | NAD(P) binding | NAD(P) binding | Alcohol/glucose |
| | | | Rossman | Rossman | dehydrog. |
| Alcohol Dehydrogenase | Horse | 1deh | NAD(P) binding | NAD(P) binding | Alcohol/glucose |
| | Liver | | Rossman | Rossman | dehydrog. |
| Alcohol Dehydrogenase | Human | 1d1s | NAD(P) binding | NAD(P) binding | Alcohol/glucose |
| | | | Rossman | Rossman | dehydrog. |
| Alcohol Dehydrogenase | human | 1hdx | NAD(P) binding | NAD(P) binding | Alcohol/glucose |
| | | | Rossman | Rossman | dehydrog. |
| Alcohol Dehydrogenase | human | 1hdy | NAD(P) binding | NAD(P) binding | Alcohol/glucose |
| | | | Rossman | Rossman | dehydrog. |
| Alcohol Dehydrogenase | Horse | 1hdz | NAD(P) binding | NAD(P) binding | Alcohol/glucose |
| | Liver | | Rossman | Rossman | dehydrog. |
| Alcohol Dehydrogenase | Horse | 1hld | NAD(P) binding | NAD(P) binding | Alcohol/glucose |
| | Liver | | Rossman | Rossman | dehydrog. |
| Alcohol Dehydrogenase | human | 1htb | NAD(P) binding | NAD(P) binding | Alcohol/glucose |
| | | | Rossman | Rossman | dehydrog. |
| Alcohol Dehydrogenase | Cod | 1kev | NAD(P) binding | NAD(P) binding | Alcohol/glucose |
| | liver | | Rossman | Rossman | dehydrog. |
| Alcohol Dehydrogenase | Horse | 1lde | NAD(P) binding | NAD(P) binding | Alcohol/glucose |
| | Liver | | Rossman | Rossman | dehydrog. |
| Alcohol Dehydrogenase | horse | 1ldy | NAD(P) binding | NAD(P) binding | Alcohol/glucose |
| | liver | | Rossman | Rossman | dehydrog. |
| Alcohol Dehydrogenase | human | 1teh | NAD(P) binding | NAD(P) binding | Alcohol/glucose |
| | | | Rossman | Rossman | dehydrog. |
| Alcohol Dehydrogenase | Thermo- | 1ykf | NAD(P) binding | NAD(P) binding | Alcohol/glucose |
| | anaerobium | | Rossman | Rossman | dehydrog. |
| Alcohol Dehydrogenase | Horse | 2ohx | NAD(P) binding | NAD(P) binding | Alcohol/glucose |
| | Liver | | Rossman | Rossman | dehydrog. |
| Alcohol Dehydrogenase | Horse | 2oxi | NAD(P) binding | NAD(P) binding | Alcohol/glucose |
| | Liver | | Rossman | Rossman | dehydrog. |
| Alcohol Dehydrogenase | Horse | 3bto | NAD(P) binding | NAD(P) binding | Alcohol/glucose |
| | Liver | | Rossman | Rossman | dehydrog. |
| Alcohol Dehydrogenase | human | 3hud | NAD(P) binding | NAD(P) binding | Alcohol/glucose |
| | | | Rossman | Rossman | dehydrog. |
| D-2-hydroxyisocaproate | Lactobacillus | 1dxy | NAD(P) binding | NAD(P) binding | Formate/glycerate |
| Dehydrogenase | Casei | | Rossman | Rossman | dehydrog. |
| D-3-Phosphoglycerate | E. Coli | 1psd | NAD(P) binding | NAD(P) binding | Formate/glycerate |
| Dehydrogenase | | | Rossman | Rossman | dehydrog. |
| Dihydrodipicolinate | E. Coli | 1arz | NAD(P) binding | NAD(P) binding | Glyceraldehyde-3- |
| Reductase | | | Rossman | Rossman | phosphate hydrog. |
| Dihydrodipicolinate | E. Coli | 1dih | NAD(P) binding | NAD(P) binding | Glyceraldehyde-3- |
| Reductase | | | Rossman | Rossman | phosphate hydrog. |
| Formate Dehydrogenase | Pyrobaculum | 1qp8 | NAD(P) binding | NAD(P) binding | Formate/glycerate |
| | Aerophilum | | Rossman | Rossman | dehydrog. |
| Formate Dehydrogenase | Methylotrophic | 2nad | NAD(P) binding | NAD(P) binding | Formate/glycerate |
| | Pseudomonas | | Rossman | Rossman | dehydrog. |
| L-2-hydroxyisocaproate | Lactobacillus | 1hyh | NAD(P) binding | NAD(P) binding | Formate/glycerate |
| dehydrogenase | Confusus | | Rossman | Rossman | dehydrog. |
| L-Alanine | Phormidium | 1pjc | NAD(P) binding | NAD(P) binding | Formate/glycerate |
| Dehydrogenase | Lapideum | | Rossman | Rossman | dehydrog. |
| L-Lactate | Plasmodium | 1ldg | NAD(P) binding | NAD(P) binding | Lactate & malate |
| Dehydrogenase | Falciparum | | Rossman | Rossman | dehydrog. (N-term) |
| L-Lactate | Bacillus | 1ldl | NAD(P) binding | NAD(P) binding | Lactate & malate |
| Dehydrogenase | Delbreuckii | | Rossman | Rossman | dehydrog. (N-term) |
| L-Lactate | B. Steario- | 1ldn | NAD(P) binding | NAD(P) binding | Lactate & malate |
| Dehydrogenase | thermophilus | | Rossman | Rossman | dehydrog. (N-term) |
| L-Lactate | Bifidobacterium | 1lld | NAD(P) binding | NAD(P) binding | Lactate & malate |
| Dehydrogenase | Longum | | Rossman | Rossman | dehydrog. (N-term) |
| L-Lactate | Bifidobacterium | 1lth | NAD(P) binding | NAD(P) binding | Lactate & malate |
| Dehydrogenase | Longum | | Rossman | Rossman | dehydrog. (N-term) |
| L-Lactate | B. Steario- | 2ldb | NAD(P) binding | NAD(P) binding | Lactate & malate |
| Dehydrogenase | thermophilus | | Rossman | Rossman | dehydrog. (N-term) |
| L-Lactate | Pig | 9ldb | NAD(P) binding | NAD(P) binding | Lactate & malate |
| Dehydrogenase | Muscle | | Rossman | Rossman | dehydrog. (N-term) |
| L-Lactate | Pig | 9ldt | NAD(P) binding | NAD(P) binding | Lactate & malate |
| Dehydrogenase | Muscle | | Rossman | Rossman | dehydrog. (N-term) |
| Malate Dehydrogenase | Aquaspirillum | 1b8u | NAD(P) binding | NAD(P) binding | Lactate & malate |
| | Arcticum | | Rossman | Rossman | dehydrog. (N-term) |
| Malate Dehydrogenase | Thermus | 1bmd | NAD(P) binding | NAD(P) binding | Lactate & malate |
| | Flavis | | Rossman | Rossman | dehydrog. (N-term) |
| Malate Dehydrogenase | E. Coli | 1cme | NAD(P) binding | NAD(P) binding | Lactate & malate |
| | | | Rossman | Rossman | dehydrog. (N-term) |
| Malate Dehydrogenase | E. Coli | 1emd | NAD(P) binding | NAD(P) binding | Lactate & malate |
| | | | Rossman | Rossman | dehydrog. (N-term) |
| Malate Dehydrogenase | Haloarcula | 1hlp | NAD(P) binding | NAD(P) binding | Lactate & malate |
| | Marismortui | | Rossman | Rossman | dehydrog. (N-term) |
| Malate Dehydrogenase | Pig | 4mdh | NAD(P) binding | NAD(P) binding | Lactate & malate |
| | Heart | | Rossman | Rossman | dehydrog. (N-term) |
| Malate Dehydrogenase | Pig | 5mdh | NAD(P) binding | NAD(P) binding | Lactate & malate |
| | Heart | | Rossman | Rossman | dehydrog. (N-term) |
| Malic Enzyme | human | 1qr6 | NAD(P) binding | NAD(P) binding | Amino-acid |
| | | | Rossman | Rossman | dehydrog (C-term) |
| S-AdenosylHomocysteine | Rat | 1b3r | NAD(P) binding | NAD(P) binding | Formate/glycerate |
| Hydrolase | | | Rossman | Rossman | dehydrog. |
| Tetrahydrofolate | Human | 1a4i | NAD(P) binding | NAD(P) binding | Amino-acid |
| Dehydrogenase | | | Rossman | Rossman | dehydrog (C-term) |

Family 2: NAD(P) Rossman Binding Domain (Syn)

| | | | | | |
|---|---|---|---|---|---|
| Glutamate | Bovine | 1ch6 | NAD(P) binding | NAD(P) binding | Amino-acid |
| Dehydrogenase | Liver | | Rossman | Rossman | dehydrog (C-term) |
| Glyceraldehyde-3- | Leishmania | 1a7k | NAD(P) binding | NAD(P) binding | Glyceraldehydes-3- |
| phosphate | Mexicana | | Rossman | Rossman | phosphate |
| Dehydrogenase | | | | | dehydrog. (N-term) |
| Glyceraldehyde-3- | Thermus | 1cer | NAD(P) binding | NAD(P) binding | Glyceraldehydes-3- |
| phosphate | aquaticus | | Rossman | Rossman | phosphate |
| Dehydrogenase | | | | | dehydrog. (N-term) |
| Glyceraldehyde-3- | B. Stearo- | 1dbv | NAD(P) binding | NAD(P) binding | Glyceraldehydes-3- |
| phosphate | thermophilus | | Rossman | Rossman | phosphate |
| Dehydrogenase | | | | | dehydrog. (N-term) |
| Glyceraldehyde-3- | E. Coli | 1gad | NAD(P) binding | NAD(P) binding | Glyceraldehydes-3- |
| phosphate | | | Rossman | Rossman | phosphate |
| Dehydrogenase | | | | | dehydrog. (N-term) |
| Glyceraldehyde-3- | E. Coli | 1gae | NAD(P) binding | NAD(P) binding | Glyceraldehydes-3- |
| phosphate | | | Rossman | Rossman | phosphate |
| Dehydrogenase | | | | | dehydrog. (N-term) |
| Glyceraldehyde-3- | B. Stearo- | 1gd1 | NAD(P) binding | NAD(P) binding | Glyceraldehydes-3- |
| phosphate | thermophilus | | Rossman | Rossman | phosphate |
| Dehydrogenase | | | | | dehydrog. (N-term) |
| Glyceraldehyde-3- | Trypanosoma | 1gga | NAD(P) binding | NAD(P) binding | Glyceraldehydes-3- |
| phosphate | Brucei | | Rossman | Rossman | phosphate |
| Dehydrogenase | Brucei | | | | dehydrog. (N-term) |
| Glyceraldehyde-3- | Leishmania | 1gyp | NAD(P) binding | NAD(P) binding | Glyceraldehydes-3- |
| phosphate | Mexicana | | Rossman | Rossman | phosphate |
| Dehydrogenase | | | | | dehydrog. (N-term) |
| Glyceraldehyde-3- | Thermatoga | 1hdg | NAD(P) binding | NAD(P) binding | Glyceraldehydes-3- |
| phosphate | Marinata | | Rossman | Rossman | phosphate |
| Dehydrogenase | | | | | dehydrog. (N-term) |
| Glyceraldehyde-3- | Palinurus | 1szj | NAD(P) binding | NAD(P) binding | Glyceraldehydes-3- |
| phosphate | Versicolor | | Rossman | Rossman | phosphate |
| Dehydrogenase | | | | | dehydrog. (N-term) |
| Glyceraldehyde-3- | B. Stearo- | 2dbv | NAD(P) binding | NAD(P) binding | Glyceraldehydes-3- |
| phosphate | thermophilus | | Rossman | Rossman | phosphate |
| Dehydrogenase | | | | | dehydrog. (N-term) |
| Glyceraldehyde-3- | B. Stearo- | 3dbv | NAD(P) binding | NAD(P) binding | Glyceraldehydes-3- |
| phosphate | thermophilus | | Rossman | Rossman | phosphate |
| Dehydrogenase | | | | | dehydrog. (N-term) |
| L-3-Hydroxyacyl COA | Human | 2hdh | NAD(P) binding | NAD(P) binding | 6-phosphogluconate |
| Dehydrogenase | Heart | | Rossman | Rossman | dehydrog. (N-term) |
| Dehdrogenase | | | | | |
| Phenylalanine | Rhodococcus | 1bxg | NAD(P) binding | NAD(P) binding | Amino-acid |
| Dehydrogenase | Sp. | | Rossman | Rossman | dehydrog (C-term) |

Family 3: NAD(P) Rossman Binding Domain (Syn) Tyrosine Depependent Oxidoreductases

| | | | | | |
|---|---|---|---|---|---|
| 17β-Hydroxysteroid | Human | 1a27 | NAD(P) binding | NAD(P) binding | Tyrosine- |
| Dehydrogenase | | | Rossman | Rossman | dependent |
| 2α-20β-Hydroxysteroid | Strep. | 2hsd | NAD(P) binding | NAD(P) binding | Tyrosine- |
| Dehydrogenase | Hydrogenans | | Rossman | Rossman | dependent |
| 7α-Hydroxysteroid | E. Coli | 1ahh | NAD(P) binding | NAD(P) binding | Tyrosine- |
| Dehydrogenase | | | Rossman | Rossman | dependent |
| 7α-Hydroxysteroid | E. Coli | 1ahi | NAD(P) binding | NAD(P) binding | Tyrosine- |
| Dehydrogenase | | | Rossman | Rossman | dependent |
| 7α-Hydroxysteroid | E. Coli | 1fmc | NAD(P) binding | NAD(P) binding | Tyrosine- |
| Dehydrogenase | | | Rossman | Rossman | dependent |
| Carbonyl Reductase | Mouse | 1cyd | NAD(P) binding | NAD(P) binding | Tyrosine- |
| | | | Rossman | Rossman | dependent |
| Cis-Biphenyl-2,3- | Pseudomonas | 1bdb | NAD(P) binding | NAD(P) binding | Tyrosine- |
| Dihydrodiol-2,3- | sp. | | Rossman | Rossman | dependent |
| Dehydrogenase | | | | | |
| Dihydropteridine | Rat | 1dir | NAD(P) binding | NAD(P) binding | Tyrosine- |
| Reductase | Liver | | Rossman | Rossman | dependent |
| Dihydropteridine | Human | 1hdr | NAD(P) binding | NAD(P) binding | Tyrosine- |
| Reductase | | | Rossman | Rossman | dependent |
| Enoyl Acyl Carrier | M. | 1bvr | NAD(P) binding | NAD(P) binding | Tyrosine- |
| Protein Reductase | Tuberculosis | | Rossman | Rossman | dependent |
| Enoyl Acyl Carrier | Brassica | 1cwu | NAD(P) binding | NAD(P) binding | Tyrosine- |
| Protein Reductase | Napus (rape) | | Rossman | Rossman | dependent |
| Enoyl Acyl Carrier | E. Coli | 1dfg | NAD(P) binding | NAD(P) binding | Tyrosine- |
| Protein Reductase | | | Rossman | Rossman | dependent |
| Enoyl Acyl Carrier | E. Coli | 1dfh | NAD(P) binding | NAD(P) binding | Tyrosine- |
| Protein Reductase | | | Rossman | Rossman | dependent |
| Enoyl Acyl Carrier | E. Coli | 1dfi | NAD(P) binding | NAD(P) binding | Tyrosine- |
| Protein Reductase | | | Rossman | Rossman | dependent |
| Enoyl Acyl Carrier | Myobacterium | 1eny | NAD(P) binding | NAD(P) binding | Tyrosine- |
| Protein Reductase | Tuberculosis | | Rossman | Rossman | dependent |
| Enoyl Acyl Carrier | Mybacterium | 1enz | NAD(P) binding | NAD(P) binding | Tyrosine- |
| Protein Reductase | Tuberculosis | | Rossman | Rossman | dependent |
| Enoyl Acyl Carrier | E. Coli | 1qg6 | NAD(P) binding | NAD(P) binding | Tyrosine- |
| Protein Reductase | | | Rossman | Rossman | dependent |
| Enoyl Acyl Carrier | Common | 1qsg | NAD(P) binding | NAD(P) binding | Tyrosine- |
| Protein Reductase | Bacteria | | Rossman | Rossman | dependent |
| GDP-Fucose Synthase | E. Coli | 1bsv | NAD(P) binding | NAD(P) binding | Tyrosine- |
| | | | Rossman | Rossman | dependent |
| Sepiapterin Reductase | E. Coli | 1nas | NAD(P) binding | NAD(P) binding | Tyrosine- |
| | | | Rossman | Rossman | dependent |
| Sepiapterin Reductase | mouse | 1sep | NAD(P) binding | NAD(P) binding | Tyrosine- |
| | | | Rossman | Rossman | dependent |
| Trihydroxynaphthalene | Rice | 1ybv | NAD(P) binding | NAD(P) binding | Tyrosine- |
| Reductase | Fungus | | Rossman | Rossman | dependent |
| Tropinone Reductase-I | Jimson | 1ae1 | NAD(P) binding | NAD(P) binding | Tyrosine- |
| | Weed | | Rossman | Rossman | dependent |
| Tropinone Reductase-II | Jimsonweed | 2ae2 | NAD(P) binding | NAD(P) binding | Tyrosine- |
| | | | Rossman | Rossman | dependent |
| UDP-Galactose | E. Coli | 1a9y | NAD(P) binding | NAD(P) binding | Tyrosine- |
| Epimerase | | | Rossman | Rossman | dependent |
| UDP-Galactose | E. Coli | 1a9z | NAD(P) binding | NAD(P) binding | Tyrosine- |
| Epimerase | | | Rossman | Rossman | dependent |
| UDP-Galactose | E. Coli | 1kvq | NAD(P) binding | NAD(P) binding | Tyrosine- |
| Epimerase | | | Rossman | Rossman | dependent |
| UDP-Galactose | E. Coli | 1kvr | NAD(P) binding | NAD(P) binding | Tyrosine- |
| Epimerase | | | Rossman | Rossman | dependent |
| UDP-Galactose | E. Coli | 1kvs | NAD(P) binding | NAD(P) binding | Tyrosine- |
| Epimerase | | | Rossman | Roasman | dependent |
| UDP-Galactose | E. Coli | 1kvt | NAD(P) binding | NAD(P) binding | Tyrosine- |
| Epimerase | | | Rossman | Rossman | dependent |
| UDP-Galactose | E. Coli | 1kvu | NAD(P) binding | NAD(P) binding | Tyrosine- |
| Epimerase | | | Rossman | Rossman | dependent |
| UDP-Galactose | E. Coli | 1nai | NAD(P) binding | NAD(P) binding | Tyrosine- |
| Epimerase | | | Rossman | Rossman | dependent |
| UDP-Galactose | E. Coli | 1uda | NAD(P) binding | NAD(P) binding | Tyrosine- |
| Epimerase | | | Rossman | Rossman | dependent |
| UDP-Galactose | E. Coli | 1udb | NAD(P) binding | NAD(P) binding | Tyrosine- |
| Epimerase | | | Rossman | Rossman | dependent |
| UDP-Galactose | E. Coli | 1udc | NAD(P) binding | NAD(P) binding | Tyrosine- |
| Epimerase | | | Rossman | Rossman | dependent |
| UDP-Galactose | E. Coli | 1xel | NAD(P) binding | NAD(P) binding | Tyrosine- |
| Epimerase | | | Rossman | Rossman | dependent |
| 3α, 20 β- | Strep. | 2hsd | NAD(P) binding | NAD(P) binding | Tyrosine- |
| hydroxysteroid | Hydrogenas | | Rossman | Rossman | dependent |
| dehydrogenase | | | | | |
| 17-β hydroxy steroid | Human | 1fdu | NAD(P) binding | NAD(P) binding | Tyrosine- |
| Dehydr. | | | Rossman | Rossman | dependent |
| 17-β hydroxy steroid | Human | 1fdv | NAD(P) binding | NAD(P) binding | Tyrosine- |
| Dehydr. | | | Rossman | Rossman | dependent |
| Family 4: Catalases | | | | | |
| Catalase | Proteus | 2cah | Heme linked | Heme linked | Heme linked |
| | Mirabilis | | catalase | catalase | catalase |
| Catalase | cow | 7cat | Heme linked | Heme linked | Heme linked |
| | Liver | | catalase | catalase | catalase |
| Catalase | cow | 8cat | Heme linked | Heme linked | Heme linked |
| | Liver | | catalase | catalase | catalase |
| Family 5: β-α TIM Barrel | | | | | |
| 2,5-Diketo-D-Gluconic | Cornybacterium | 1a80 | β-α TIM Barrel | NAD(P)-linkded | Aldo-keto |
| Acid Reductase | sp | | | Oxidoreductase | Reductase |
| 3-α-hydroxysteroid | Rat | 1afs | β-α TIM Barrel | NAD(P)-linkded | Aldo-keto |
| Dehydrogenase | | | | Oxidoreductase | Reductase |
| Aldehyde Reductase | Pig | 1ae4 | β-α TIM Barrel | NAD(P)-linkded | Aldo-keto |
| | | | | Oxidoreductase | Reductase |
| Aldehyde Reductase | Pig | 1cwn | β-α TIM Barrel | NAD(P)-linkded | Aldo-keto |
| | | | | Oxidoreductase | Reductase |
| Aldo-keto Reductase | Mouse | 1frb | β-α TIM Barrel | NAD(P)-linkded | Aldo-keto |
| | | | | Oxidoreductase | Reductase |
| Aldose Reductase | Human | 1abn | β-α TIM Barrel | NAD(P)-linkded | Aldo-keto |
| | | | | Oxidoreductase | Reductase |
| Aldose Reductase | Human | 1ads | β-α TIM Barrel | NAD(P)-linkded | Aldo-keto |
| | | | | Oxidoreductase | Reductase |
| Aldose Reductase | Pig | 1ah0 | β-α TIM Barrel | NAD(P)-linkded | Aldo-keto |
| | | | | Oxidoreductase | Reductase |
| Aldose Reductase | Pig eye | 1ah3 | β-α TIM Barrel | NAD(P)-linkded | Aldo-keto |
| | | | | Oxidoreductase | Reductase |
| Aldose Reductase | Pig | 1ah4 | β-α TIM Barrel | NAD(P)-linkded | Aldo-keto |
| | | | | Oxidoreductase | Reductase |
| Aldose Reductase | Human | 1az1 | β-α TIM Barrel | NAD(P)-linkded | Aldo-keto |
| | | | | Oxidoreductase | Reductase |
| Aldose Reductase | Human | 1az2 | β-α TIM Barrel | NAD(P)-linkded | Aldo-keto |
| | | | | Oxidoreductase | Reductase |
| Aldose Reductase | Human | 1mar | β-α TIM Barrel | NAD(P)-linkded | Aldo-keto |
| | | | | Oxidoreductase | Reductase |
| Aldose Reductase | Human | 2acq | β-α TIM Barrel | NAD(P)-linkded | Aldo-keto |
| | | | | Oxidoreductase | Reductase |
| Aldose Reductase | Human | 2acr | β-α TIM Barrel | NAD(P)-linkded | Aldo-keto |
| | | | | Oxidoreductase | Reductase |
| Aldose Reductase | Human | 2acs | β-α TIM Barrel | NAD(P)-linkded | Aldo-keto |
| | | | | Oxidoreductase | Reductase |
| Aldose Reductase | Human | 2acu | β-α TIM Barrel | NAD(P)-linkded | Aldo-keto |
| | | | | Oxidoreductase | Reductase |

Family 6: Dihydrofolate Reductases

| | | | | | |
|---|---|---|---|---|---|
| Dihydrofolate | Candida | 1ai9 | Dihydrofolate | Dihydrofolate | Dihydrofolate |
| Reductase | Albicans | | Reductase | Reductase | Reductase |
| Dihydrofolate | Candida | 1aoe | Dihydrofolate | Dihydrofolate | Dihydrofolate |
| Reductase | Albicans | | Reductase | Reductase | Reductase |
| Dihydrofolate | Pneumocystis | 1daj | Dihydrofolate | Dihydrofolate | Dihydrofolate |
| Reductase | carinii | | Reductase | Reductase | Reductase |
| Dihydrofolate | Human | 1dlr | Dihydrofolate | Dihydrofolate | Dihydrofolate |
| Reductase | | | Reductase | Reductase | Reductase |
| Dihydrofolate | Human | 1dls | Dihydrofolate | Dihydrofolate | Dihydrofolate |
| Reductase | | | Reductase | Reductase | Reductase |
| Dihydrofolate | Chicken | 1dr1 | Dihydrofolate | Dihydrofolate | Dihydrofolate |
| Reductase | Liver | | Reductase | Reductase | Reductase |
| Dihydrofolate | Chicken | 1dr4 | Dihydrofolate | Dihydrofolate | Dihydrofolate |
| Reductase | Liver | | Reductase | Reductase | Reductase |
| Dihydrofolate | Chicken | 1dr5 | Dihydrofolate | Dihydrofolate | Dihydrofolate |
| Reductase | Liver | | Reductase | Reductase | Reductase |
| Dihydrofolate | Chicken | 1dr6 | Dihydrofolate | Dihydrofolate | Dihydrofolate |
| Reductase | Liver | | Reductase | Reductase | Reductase |
| Dihydrofolate | Chicken | 1dr7 | Dihydrofolate | Dihydrofolate | Dihydrofolate |
| Reductase | Liver | | Reductase | Reductase | Reductase |
| Dihydrofolate | E. Coli | 1dre | Dihydrofolate | Dihydrofolate | Dihydrofolate |
| Reductase | | | Reductase | Reductase | Reductase |
| Dihydrofolate | E. Coli | 1drh | Dihydrofolate | Dihydrofolate | Dihydrofolate |
| Reductase | | | Reductase | Reductase | Reductase |
| Dihydrofolate | Pneumocystis | 1dyr | Dihydrofolate | Dihydrofolate | Dihydrofolate |
| Reductase | carinii | | Reductase | Reductase | Reductase |
| Dihydrofolate | Human | 1hfp | Dihydrofolate | Dihydrofolate | Dihydrofolate |
| Reductase | | | Reductase | Reductase | Reductase |
| Dihydrofolate | Human | 1hfq | Dihydrofolate | Dihydrofolate | Dihydrofolate |
| Reductase | | | Reductase | Reductase | Reductase |
| Dihydrofolate | Human | 1hfr | Dihydrofolate | Dihydrofolate | Dihydrofolate |
| Reductase | | | Reductase | Reductase | Reductase |
| Dihydrofolate | Human | 1ohj | Dihydrofolate | Dihydrofolate | Dihydrofolate |
| Reductase | | | Reductase | Reductase | Reductase |
| Dihydrofolate | Human | 1ohk | Dihydrofolate | Dihydrofolate | Dihydrofolate |
| Reductase | | | Reductase | Reductase | Reductase |
| Dihydrofolate | E. Coli | 1ra2 | Dihydrofolate | Dihydrofolate | Dihydrofolate |
| Reductase | | | Reductase | Reductase | Reductase |
| Dihydrofolate | E. Coli | 1rb2 | Dihydrofolate | Dihydrofolate | Dihydrofolate |
| Reductase | | | Reductase | Reductase | Reductase |
| Dihydrofolate | E. Coli | 1rh3 | Dihydrofolate | Dihydrofolate | Dihydrofolate |
| Reductase | | | Reductase | Reductase | Reductase |
| Dihydrofolate | E. Coli | 1rx1 | Dihydrofolate | Dihydrofolate | Dihydrofolate |
| Reductase | | | Reductase | Reductase | Reductase |
| Dihydrofolate | E. Coli | 1rx2 | Dihydrofolate | Dihydrofolate | Dihydrofolate |
| Reductase | | | Reductase | Reductase | Reductase |
| Dihydrofolate | E. Coli | 1rx3 | Dihydrofolate | Dihydrofolate | Dihydrofolate |
| Reductase | | | Reductase | Reductase | Reductase |
| Dihydrofolate | Lactobacillus | 3dfr | Dihydrofolate | Dihydrofolate | Dihydrofolate |
| Reductase | casei | | Reductase | Reductase | Reductase |
| Dihydrofolate | E. Coli | 7dfr | Dihydrofolate | Dihydrofolate | Dihydrofolate |
| Reductase | | | Reductase | Reductase | Reductase |
| Dihydrofolate | Chicken | 8dfr | Dihydrofolate | Dihydrofolate | Dihydrofolate |
| Reductase | Liver | | Reductase | Reductase | Reductase |

Family 7: FAD/NAD(P) Binding Oxidoreductases (‘Disulfide Oxidoreductases’)

| | | | | | |
|---|---|---|---|---|---|
| Glutathione Reductase | E. Coli | 1get | FAD/NAD(P) | FAD/NAD(P) | FAD/NAD-linked |
| | | | Binding Domain | Binding Domain | reductases |
| Glutathione Reductase | E. Coli | 1geu | FAD/NAD(P) | FAD/NAD(P) | FAD/NAD-linked |
| | | | Binding Domain | Binding Domain | reductases |
| Glutathione Reductase | Human | 1grb | FAD/NAD(P) | FAD/NAD(P) | FAD/NAD-linked |
| | | | Binding Domain | Binding Domain | reductases |
| NADH Peroxidase | Streptococcus | 2npx | FAD/NAD(P) | FAD/NAD(P) | FAD/NAD-linked |
| | Faecalis | | Binding Domain | Binding Domain | reductases |
| Thioredoxin Reductase | E. Coli | 1tdf | FAD/NAD(P) | FAD/NAD(P) | FAD/NAD-linked |
| | | | Binding Domain | Binding Domain | reductases |
| Trypanothione | Crithidia | 1typ | FAD/NAD(P) | FAD/NAD(P) | FAD/NAD-linked |
| Reductase* (by active | Fasciculata | | Binding Domain | Binding Domain | reductases |
| site) | | | | | |
| Family 8: Ferrodoxin-like | | | | | |
| Ferrodoxin Reductase | Pea | 1qga | Ferrodoxin like | Ferrodoxin like | Reductases |
| P450 Reductase | Rat | — | Ferrodoxin like | Ferrodoxin like | NADPH-cytochrome |
| | | | | | P450 reductase |

The results shown in Table 11 demonstrate that bound conformation of NAD(P)(H) can be correlated with protein fold. Grouping oxidoreductases into pharmacofamilies based on the bound conformations of NAD(P)(H) resulted in a correlation with protein fold. Pharmacofamilies 1-3 consist of polypeptides having the NAD(P)(H) binding Rossman fold. Pharmacofamily 4 consists of polypeptides having heme-linked catalase fold. Pharmacofamily 5 consists of polypeptides having the β-αTIM barrel fold. Pharmacofamily 6 consists of polypeptides having the dihydrofolate reductase fold. Pharmacofamily 7 consists of polypeptides having the FAD/NAD(P)(H) binding domain fold. Trypanathione reductase was added to family 7 by homology of its active site to the active sites of other members of pharmacofamily 7 independent of bound ligand conformation. Pharmacofamily 8 consists of polypeptides having the ferrodoxin like fold. Pharmacofamilies 1 and 2 were identified based on anti or syn conformation, respectively, of the nicotinamide ring relative to the ribose. Additionally, a change in the torsion angles in the bonds connecting the adenine ribose to the adenine phosphate separates the family members having a Rossman fold into a third pharmacofamily, identified as pharmacofamily 3.

The results described in this example demonstrate that a bound conformation of a ligand can be correlated with polypeptide fold. Furthermore, the results obtained by the method are consistent with results obtained by SCOP. Therefore, classification based on bound conformation of ligands can be used to classify polypeptides according to structure.

### EXAMPLE III

### Determination of a Conformer Model and Pharmacophore for Pharmacoclusters 1-8

This example demonstrates determination of the average bound conformations from pharmacoclusters 1-8 and construction of conformer models based on the average bound conformations. This example also demonstrates construction of a pharmacophore model based on the average bound conformations and interactions with polypeptides.

Conformer models for each pharmacocluster were produced by determining an average structure for the subset of members of each pharmacocluster as described in Example I. The coordinates for conformer models of pharmacoclusters 1-8 are shown in Part C of Tables 3-10 respectively.

Pharmacophore models were constructed by aligning the active sites of a pharmacofamily of oxidoreductases. Three-dimensional overlays were achieved using Insight II overlay module to overlay the NAD(P) ligands of each enzyme-ligand complex. Heteroatoms in the surrounding protein that could function as hydrogen bond acceptors or hydrogen bond donors were identified in each complex that made interactions with the NAD(P) ligand. These heteroatoms that had common positions in three dimensional space (within 3Åof each other in the overlay) in each enzyme complex and that made a common interaction with the ligand were then grouped together and tabulated for pharmacophore construction. Water molecules were similarly identified and grouped. The grouped heteroatoms and water molecules are listed in Part D of Tables 3-10 below. Finally the average coordinates and the standard deviation for each interaction group were calculated. The final pharmacophore model was produced by overlaying interaction groups on the conformer model (average ligand structure).

The coordinates for pharmacophore models of pharmacoclusters 1-8 are shown in parts B and C of Tables 3-10, respectively. Specifically, each conformer model includes the average NAD(P) coordinates (in part C of each Table) and the pharmacophore model includes both the average NADP coordinates, average water coordinates and the average protein heteroatom coordinates (including coordinates in both part B and C of each Table). An exception is the pharmacophore model derived from pharmacofamily 7 which includes average water coordinates and average protein heteroatom coordinates for all polypeptides listed but has a conformer model derived from NAD(P) bound to each polypeptide listed except trypanathione reductase.

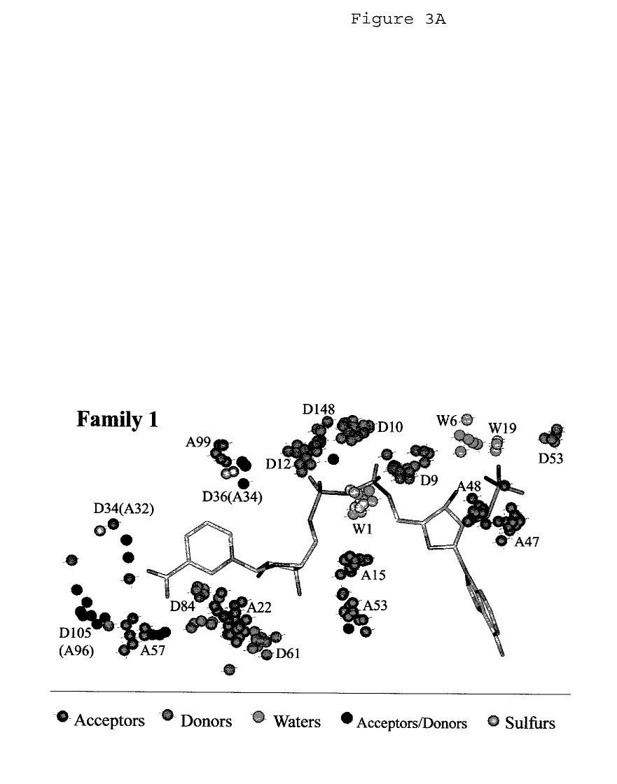

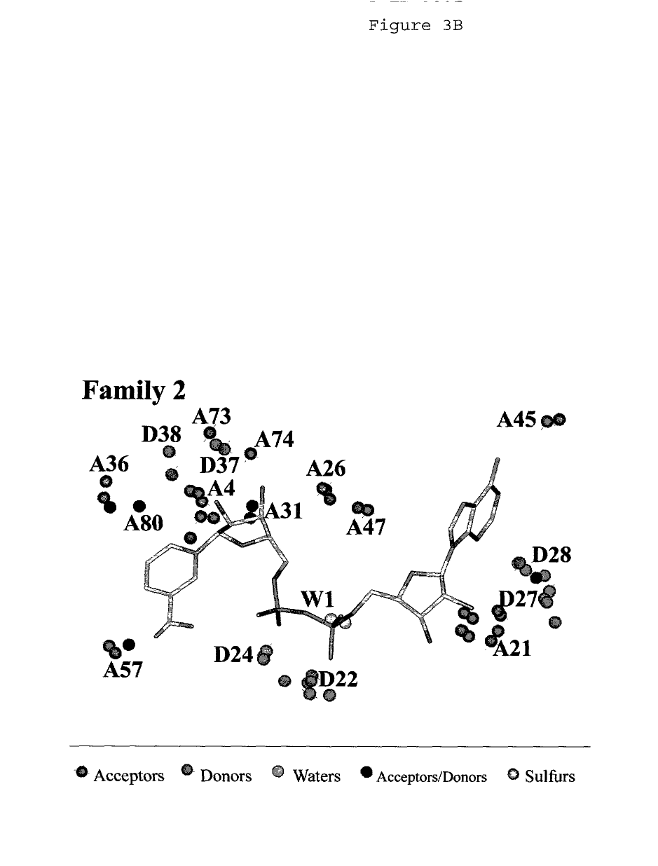

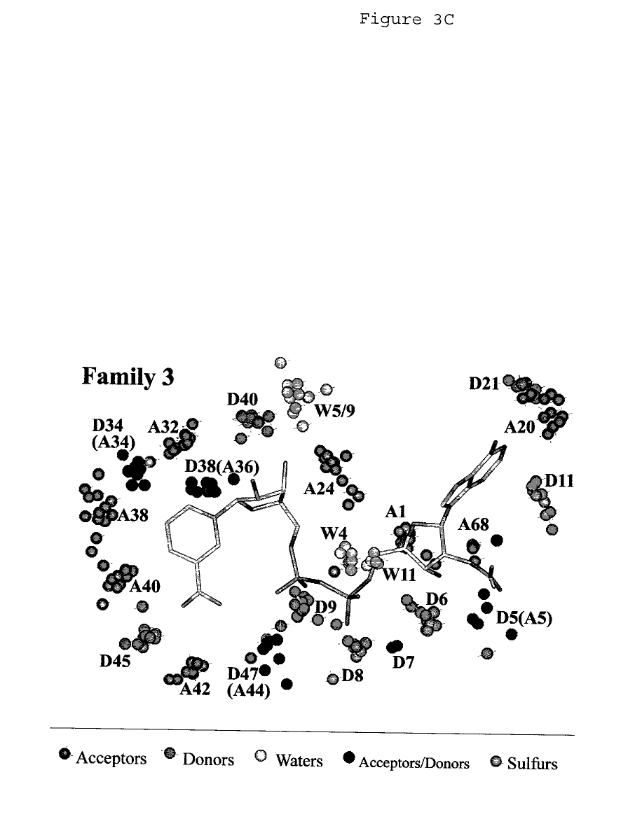

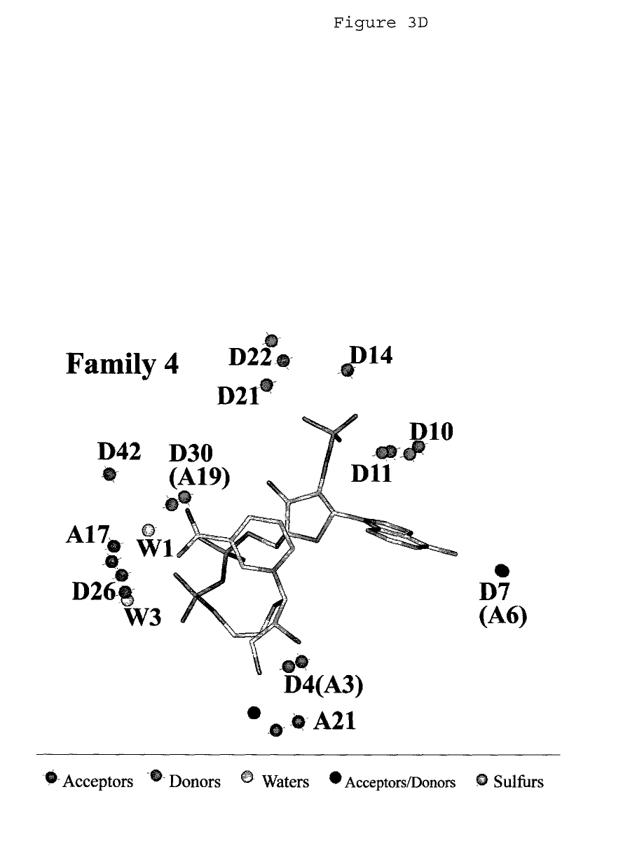

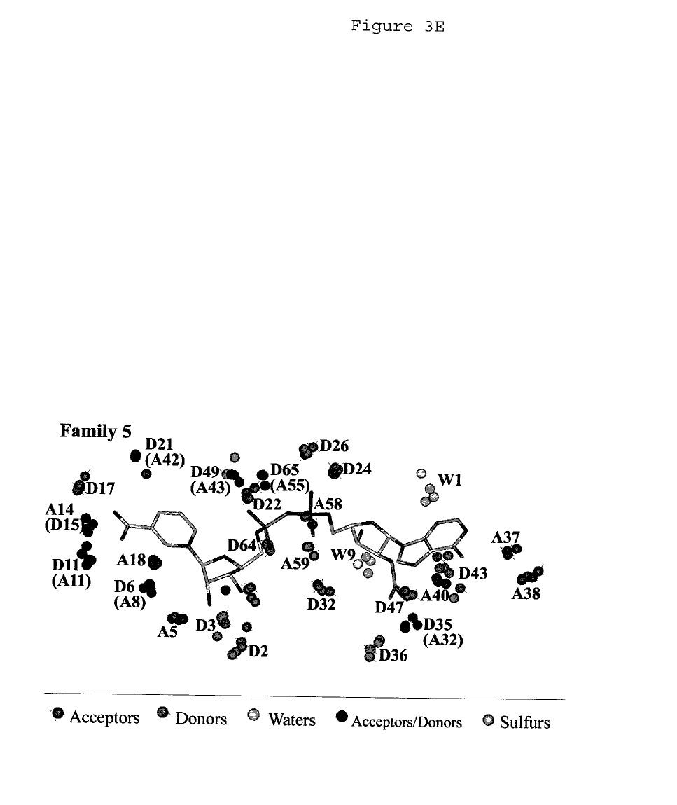

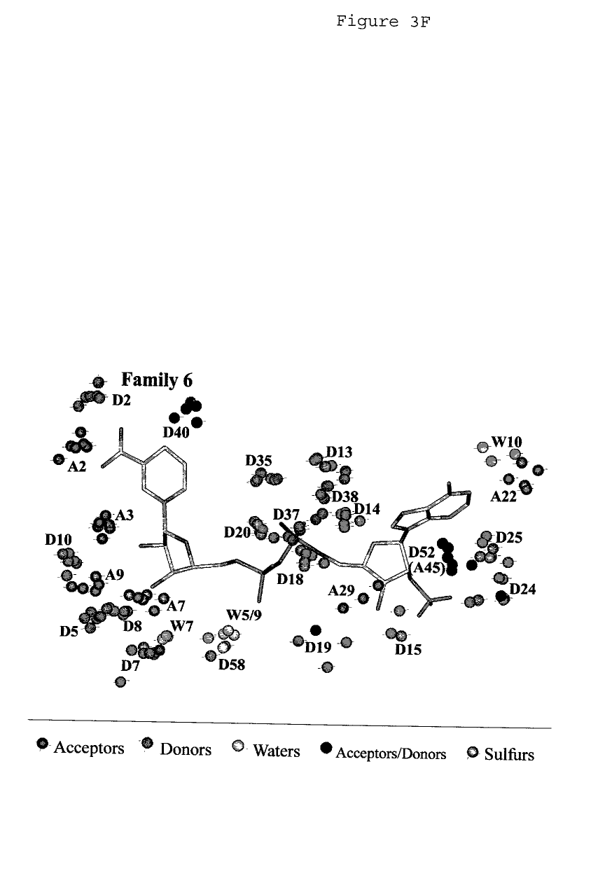

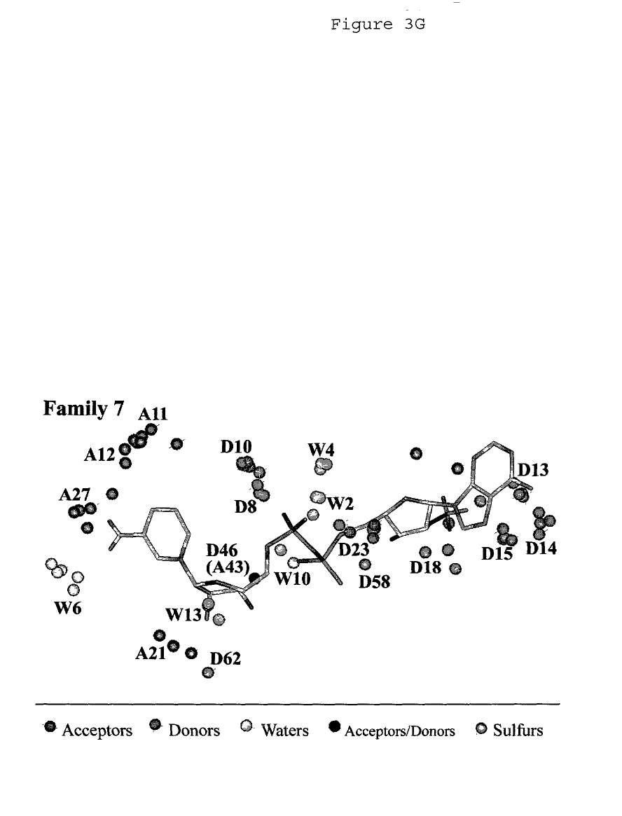

A structural representation of each conformer model with overlayed interaction groups used to determine respective pharmacophore models 1-8 is provided in FIG. 3. The structures shown in FIG. 3 reflect the average NAD(P) coordinates shown in Part C of Tables 3-10 and the coordinates for all interacting groups used to calculate the average water coordinates and the average protein heteroatom coordinates as shown in Part D of Tables 3-10. Hydrogen bond acceptors are labeled with an ‘A’ followed by a number for each group. These are listed in the pharmacophore Tables and designated on the pharmacophore figures. Donors are labeled with a ‘D’; and water molecules are labeled with a ‘W’.

This example demonstrates construction of conformer models based on the bound conformations of ligands in pharmacoclusters. This example also demonstrates construction of a pharmacophore model based on the bound conformations of ligands in pharmacoclusters and their interactions with polypeptides in their respective pharmacofamilies.

### EXAMPLE IV

### Correlation Between the Bound Conformation of Ligands and a Conformation-Dependent Property

This example describes a conformation-dependent property that is correlated with a bound conformation of a ligand.

A 2D [¹H,¹H] NOESY spectrum was recorded with a 0.2 ml sample of 1 mM NADP and 200 μM of enzyme 1-deoxy D-xylulose 5-phosphate reductoisomerase (DOXP). The spectrum was measured with a Bruker DRX700 spectrometer operating at 700 MHZ ¹H frequency. The total measuring time was about 12 h.

The spectrum is shown in FIG. 4 and atoms are identified according to FIG. 2. The relative intensities of the observed transferred NOEs (trNOEs) between the ribose proton H-C1′N(NC1′) and the protons on the nicotinamide ring, H-C4N and H-C2N shown in FIG. 4, reveal that the NADP adopts a syn conformation when bound to the enzyme.

The bound conformations in Pharmacocluster 1 and 2 can be distinguished according to anti or syn conformation, respectively, of the nicotinamide ring relative to the ribose. Therefore, these results demonstrate that the relative intensities of the observed trNOE's between the ribose proton H-C1′N(NC1′) and the protons on the nicotinamide ring, H-C4N and H-C2N can provide a conformation dependent property useful in distinguishing members of pharmacoclusters 1 and 2.

### EXAMPLE V

### Binding Compounds Having Specificity for One or More Polypeptide Pharmacofamilies

This example demonstrates querying a database of compounds to identify individual compounds having similar conformations. This example also demonstrates preferential binding of a compound to a polypeptide of one pharmacofamily over another.

The TTE0001.001.A07 AND TTE0001.002.D02 compounds were identified by using the THREEDOM algorithm to query a database of commercially available molecules (ASINEX; Moscow, Russia) by shape matching with cibacron blue. Coordinates of cibacron blue were obtained from the published 3D structure (Li et al., Proc. Natl. Acad. Sci. USA 92:8846-8850 (1995)). The database was created by converting an SD format file of structures from ASINEX to INTERCHEM format coordinates using the batch2to3 program. Cibacron blue was compared against each structure in the database in multiple orientations to generate a matching score. Out of 37,926 structures searched, the 750 best matching scores were selected. From these 750 structures, TTE0001.001.A07 AND TTE0001.002.D02 were selected and purchased based on objective criteria such as likely favorable binding interactions, pharmacophore properties, synthetic accessibility and likely pharmacokinetic, toxicological, adsorption and metabolic properties.

Kinetic studies were carried out in 1-cm cuvettes in a 1 mL volume at 25° C. Lactate dehydrogenase reactions were monitored spectrophotometrically with a Cary 300 by following the decrease in absorbance at 340 nm due to the oxidation of NADH by pyruvate. Lactate dehydrogenase reaction mixtures contained 100 mM Hepes buffer at pH 7.4, as well as 2.5 mM pyruvate, 10 μM NADH, 5 ng/mL lactate dehydrogenase. NADPH, NADH, Hepes buffer, and rabbit muscle lactate dehydrogenase were purchased from Sigma. Cytochrome P450 reductase reactions were monitored by following the decrease in absorbance at 550 nm due to the reduction of ferric cytochrome c by NADPH. Cytochrome P450 reductase reaction mixtures contained 100 mM Hepes buffer at pH 7.4, as well as 80 μM ferric cytochrome c, 10 μM NADPH, and 80 ng/mL cytochrome P450 reductase. Data were fitted using the FORTRAN programs of Cleland, Adv. Enzymol. 45: 273-387 (1977) which perform nonlinear least squares fits to the appropriate equations. Substrates were varied around their Michaelis constants, while nonvaried substrate was kept at a concentration close to its Michaelis constant. The concentration of inhibitor that gives 50% inhibition (IC50) values were obtained by fitting data to the equation for a line, where Y values are 1/rate and X values are the concentration of inhibitor, as in a Dixon plot (Segel, supra). The X-intercept is the IC50. If a full kinetic profile was done, then Kᵢₛ values were obtained by fitting the data to the equation for a competitive inhibitor:

$$rate = \frac{{V}_{max} A}{{K}_{m} ( 1 + I / {K}_{is} ) + A}$$

where rate is the rate of reaction in units of absorbance/minute, Vₘₐₓ is the maximum velocity, Kₘ is the Michaelis constant for A, Kᵢₛ is the inhibition dissociation constant for the inhibitor, I is the inhibitor concentration, and A is the concentration of NADH or NADPH. In all cases, the fit to the above equation was used only after establishing that the fit to equations for noncompetitive and uncompetitive inhibition were less appropriate based on values for sigma (overall fit) as well as standard deviations for fitted constants (Kᵢₛ and Kᵢᵢ).

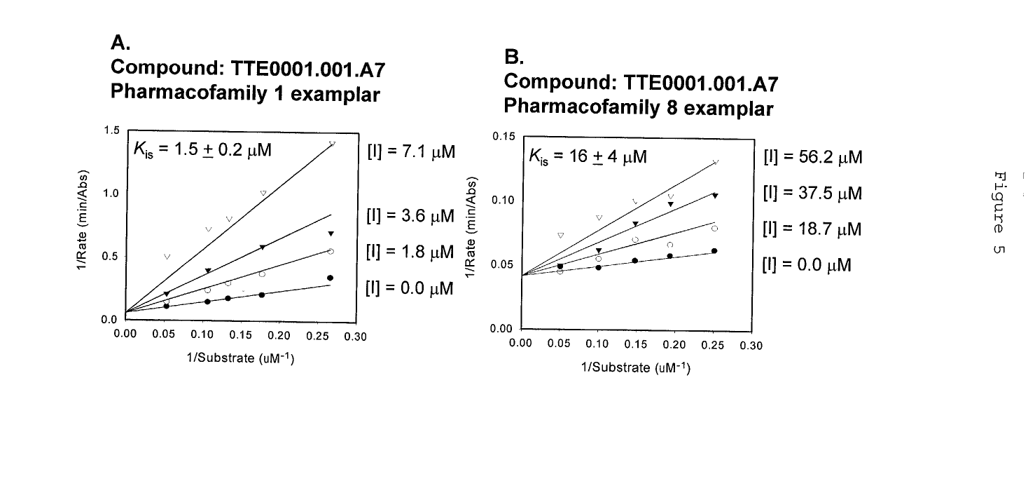

As shown in FIG. 5, compound TTE0001.001.A07 could inhibit binding of NADH to lactate dehydrogenase and NADPH to cytochrome P450 reductase which are polypeptide members of pharmacofamily 1 and 8 respectively. Compound TTE0001.001.A07 demonstrated high binding affinity for both lactate dehydrogenase and cytochrome P450 reductase.

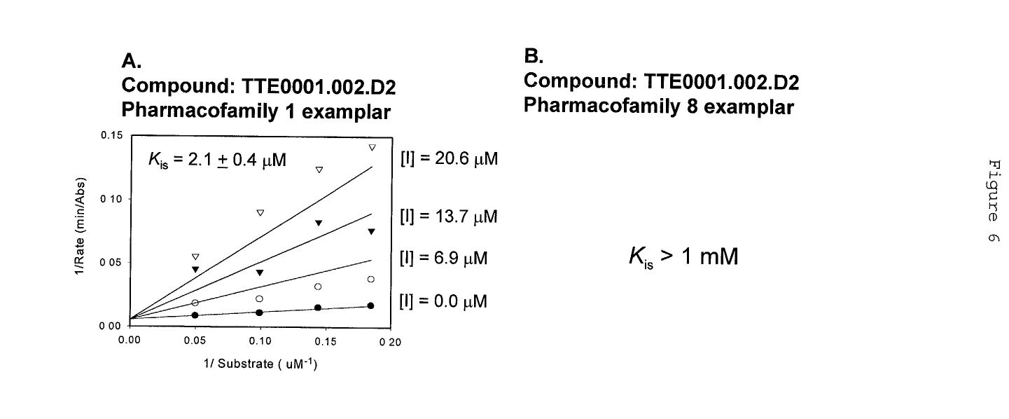

Analysis of inhibition of binding between NADH and lactate dehydrogenase is shown in FIG. 6. Compound TTE0001.002.D02 inhibited lactate dehydrogenase with a Kᵢₛ of 2.1 μM. Similar measurements of cytochrome P450 reductase with concentrations of compound TTE0001.002.D02 up to 0.5 mM did not indicate inhibition. These results indicated that compound TTE0001.002.D02 had a Kᵢₛ of greater than 1 mM with cytochrome P450 reductase. Thus, compound TTE0001.002.D02 demonstrated preferential binding for pharmacofamily 1 having an inhibitory dissociation constant (Kᵢₛ) that was at least 500 fold lower than for pharmacofamily 8.

The results described in this example demonstrate that a binding compound can be identified by structural comparison to a bound conformation of a ligand. Furthermore, the results demonstrate that binding compounds that interact with polypeptides from multiple pharmacofamilies or compounds that preferentially bind to polypeptides of one pharmacofamily compared to polypetides of another pharmacofamily can be identified by structural comparison to a bound conformation of a ligand.

### EXAMPLE VI

### Identification of a Ligand Using a Pharmacophore Model

This example demonstrates construction of a pharmacophore model, use of the model to identify a binding ligand and confirmation of the ability of the identified compound to bind a polypeptide member of the pharmacofamily from which the pharmacophore model was derived.

Pharmacophore models were constructed to include part or all of the NAD(P) shape, hydrogen bond donors, hydrogen bond acceptors and/or other chemical features described in Tables 3-10. The combination of chemical features chosen for each search pharmacophore in a search set were chosen in an attempt to cover a diverse range of combinations of possible chemical interactions and to represent the protein ligand interactions that occur most frequently in the particular pharmacofamily.

Pharmacophore shape was derived using the program CATALYST, and was calculated using the Van der Waals surface for part or all of the structure of the averaged NAD(P) coordinates determined for a pharmacocluster. Desired hydrogen bonding features, water molecules and other chemical motifs were positioned in the pharmacophore model using the average coordinates determined for both the pharmacofamily and pharmacocluster.

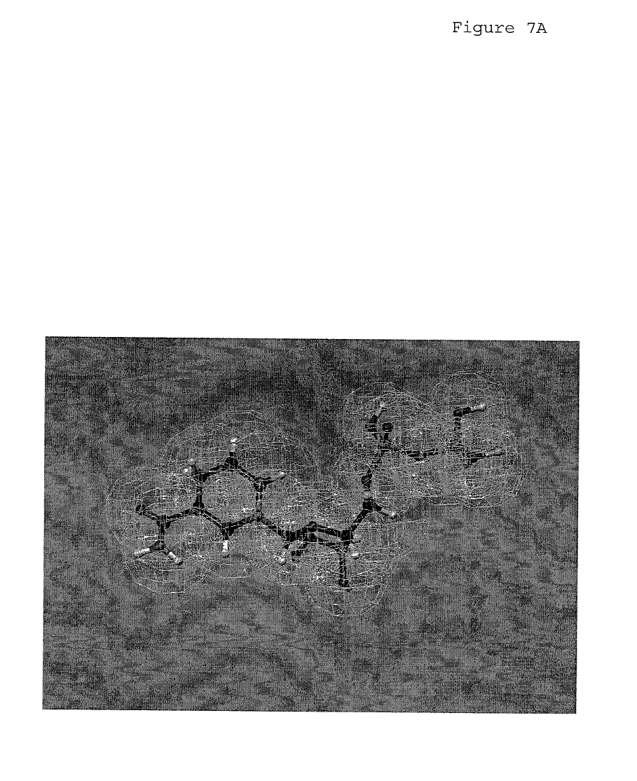

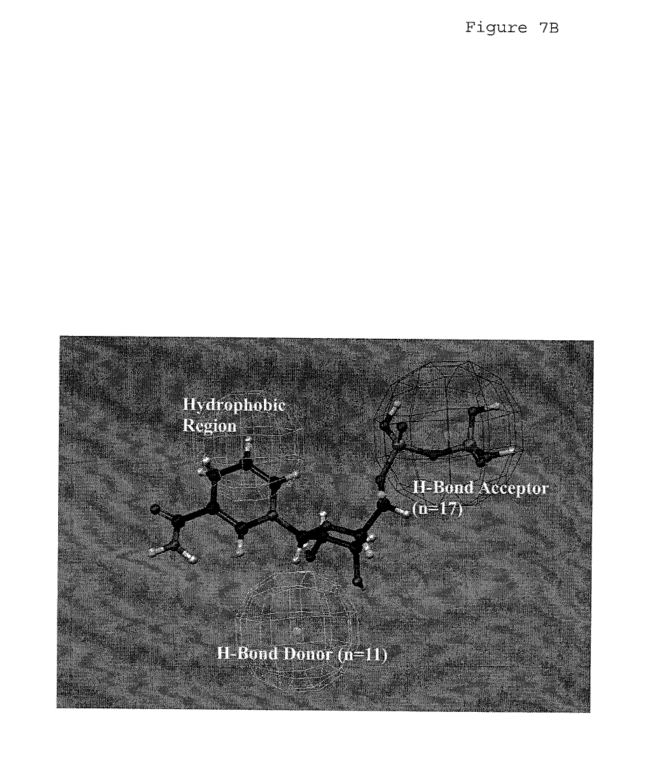

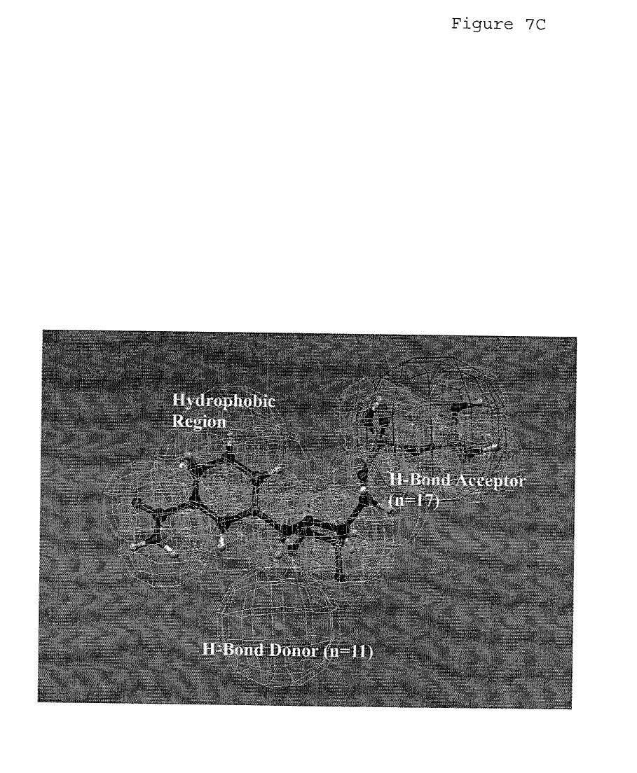

The components of a pharmacophore model derived from the coordinates presented in Table 3 for pharmacofamily 1 are shown in FIG. 7. FIG. 7A shows the structure for the conformer model having coordinates listed in Table 3C with a superimposed volume defining the shape of the ligand and indicated by grey spheres. A hydrophobic feature was added to the pharmacophore model at the average position of the hydrophobic region of the nicotinamide ring as shown in FIG. 7B. Also shown in FIG. 7B is a hydrogen bond acceptor positioned at the average coordinates for the pyrophosphate using the averaged coordinates for the location of hydrogen bond acceptors utilized in all of the 17 polypeptides of the pharmacofamily. Finally, FIG. 7B shows a hydrogen bond donor positioned according to a position where a hydrogen bond donor of a ligand would be expected to have favorable interactions with hydrogen bond acceptors observed in 11 of the polypeptides of pharmacofamily 1. Thus, the hydrogen bond donor does not identify a position of an actual hydrogen bond donor in the NAD(P) ligand, but instead a location to where a potential ligand's hydrogen bond donor could make favorable interactions with the polypeptides of pharmacofamily 1. FIG. 7C shows the combined features of FIGS. 7A and 7B present in a pharmacophore model used to search a database of compounds.

To identify potential ligands that bind to polypeptides of pharmacofamily 1, computational searches were conducted using CATALYST. Searches were made by comparing the shape and combination of chemical features of the pharmacophore model, shown in FIG. 7, to the shape and features of molecules in the database.

An example of a compound identified using the pharmacophore model shown in FIG. 7C is TTE0008.025.D08. Using a binding assay similar to that described in Example V, compound TTE0008.025.D08 was shown to have inhibitory activity against pharmacofamily 1 member, dihydrodipicolinate reductase (IC₅₀2.8 μM).

TABLE 3A
Pharmacofamily 1 Subset

| | | | RMSD from |
|---|---|---|---|
| Molecule # | pdb | type | Family Avg. |
| 1 | 1A4I | Tetrahydrofolate Reductase | 0.75 |
| | | (human) | |
| 2 | 1AXE | Alcohol Dehydrogenase (horse) | 0.27 |
| 3 | 1DXY | D2-Hydroxyisocaproate | 0.92 |
| | | Dehydrogenase (L. Casei) | |
| 4 | 1LDN | L-Lactate Dehydrogenase | 0.41 |
| | | (B. Stearothermophilus) | |
| 5 | 1QR6 | Malic Enzyme (human) | 0.77 |
| 6 | 4MDH | Malate Dehydrogenase (pig) | 0.65 |
| 7 | 1AGN | Alcohol Dehydrogenase (human | 0.63 |
| | | class IV sigma) | |
| 8 | 1B3R | Adenosylhomocysteine (rat) | 0.93 |
| 9 | 1EMD | Malate Dehydrogenase (E. Coli) | 0.90 |
| 10 | 1PJC | L-Alanine (Phormidium Lapideum) | 0.79 |
| 11 | 1YKF | Alcohol Dehydrogenase | 1.06 |
| | | (Thermoanaerobium Brockii) | |
| 12 | 9LDB | Lactate Dehydrogenase (pig) | 0.36 |
| 13 | 1ARZ | Dihydrodipicolinate Reductase | 0.81 |
| | | (E. Coli) | |
| 14 | 1BMD | Malate Dehydrogenase | 0.68 |
| | | (Thermus Flavis) | |
| 15 | 1HYH | L2-Hydroxyisocaproate | 0.57 |
| | | Dehydrogenase (Lactobacillus | |
| | | Confusus) | |
| 16 | 1PSD | D3-Phosphoglycerate | 0.78 |
| | | Dehydrogenase (E. Coli) | |
| 17 | 2NAD | Formate Dehydrogenase | 0.91 |
| | | (methylotrophic bacterium | |
| | | pseudomonas sp 101) | |

TABLE 3B
Polypeptide and Solvent Interactors (average coordinates)

| atom | | | | | | | | |
|---|---|---|---|---|---|---|---|---|
| name | name | total | x | σx | y | σy | z | σz |
| A15 | ACC | 15 | −3.51 | 0.52 | −1.48 | 0.44 | −4.24 | 0.49 |
| A22 | ACC | 17 | 3.14 | 0.41 | −2.17 | 0.33 | −4.13 | 1.01 |
| A32 | ACC | 5 | 7.37 | 0.45 | 1.75 | 1.11 | −8.24 | 0.79 |
| A34 | ACC | 6 | 1.20 | 0.42 | 6.08 | 0.33 | −1.83 | 1.39 |
| A47 | ACC | 13 | −12.03 | 0.32 | −1.22 | 0.56 | −3.63 | 0.52 |
| A48 | ACC | 14 | −10.58 | 0.37 | −0.79 | 0.39 | −4.81 | 0.25 |
| A53 | ACC | 11 | −2.66 | 0.31 | −2.95 | 0.58 | −1.04 | 0.46 |
| A57 | ACC | 11 | 7.56 | 0.73 | −2.50 | 0.42 | −6.36 | 0.45 |
| A96 | ACC | 6 | 10.24 | 0.42 | 0.50 | 0.64 | −2.97 | 0.32 |
| A99 | ACC | 4 | 1.44 | 0.22 | 6.19 | 0.26 | −5.24 | 0.38 |
| D9 | DON | 17 | −7.70 | 0.67 | 2.30 | 0.43 | −6.27 | 0.29 |
| D10 | DON | 17 | −5.49 | 0.58 | 5.00 | 0.44 | −5.79 | 0.28 |
| D12 | DON | 17 | −3.06 | 0.53 | 4.22 | 0.42 | −7.05 | 0.38 |
| D34 | DON | 2 | 7.05 | 0.16 | 1.64 | 0.42 | −7.81 | 0.74 |
| D36 | DON | 4 | 1.28 | 0.39 | 6.13 | 0.37 | −1.01 | 0.70 |
| D53 | DON | 5 | −14.97 | 0.29 | 3.01 | 0.15 | −1.95 | 0.55 |
| D61 | DON | 11 | 2.46 | 0.64 | −2.82 | 0.54 | −0.35 | 0.58 |
| D84 | DON | 11 | 4.78 | 0.45 | 0.00 | 0.90 | −0.25 | 0.46 |
| D105 | DON | 7 | 10.22 | 0.38 | 0.54 | 0.59 | −3.10 | 0.45 |
| D148 | DON | 4 | −3.98 | 0.86 | 7.02 | 0.14 | −1.61 | 0.33 |
| W1 | WAT | 14 | −4.88 | 0.34 | 1.26 | 0.38 | −5.81 | 0.27 |
| W6 | WAT | 6 | −10.83 | 0.37 | 3.79 | 0.41 | −3.11 | 0.70 |
| W19 | WAT | 3 | −12.43 | 0.10 | 2.22 | 0.31 | −5.57 | 0.42 |

TABLE 3C
NAD(P) Conformer Model

| atom name | total | x | σx | y | σy | z | σz |
|---|---|---|---|---|---|---|---|
| PA | 17 | −5.47 | 0.22 | 3.43 | 0.30 | −1.84 | 0.27 |
| O2A | 17 | −5.82 | 0.31 | 4.60 | 0.37 | −2.38 | 0.65 |
| O1A | 17 | −5.72 | 0.50 | 3.38 | 0.60 | −0.59 | 0.64 |
| O5′A | 17 | −6.13 | 0.25 | 2.22 | 0.25 | −2.57 | 0.37 |
| C5′A | 17 | −6.23 | 0.13 | 0.92 | 0.22 | −2.20 | 0.23 |
| C4′A | 17 | −7.50 | 0.39 | 0.21 | 0.43 | −2.82 | 0.24 |
| O4′A | 17 | −7.46 | 0.19 | −1.07 | 0.14 | −2.48 | 0.34 |
| C3′A | 17 | −8.76 | 0.20 | 0.85 | 0.28 | −2.35 | 0.43 |
| O3′A | 17 | −9.62 | 0.37 | 1.13 | 0.33 | −3.41 | 0.67 |
| C2′A | 17 | −9.32 | 0.23 | −0.09 | 0.31 | −1.58 | 0.37 |
| O2′A | 17 | −10.69 | 0.36 | −0.06 | 0.51 | −1.72 | 0.54 |
| C1′A | 17 | −8.69 | 0.37 | −1.29 | 0.45 | −2.19 | 0.31 |
| N9A | 17 | −8.88 | 0.18 | −2.60 | 0.08 | −1.36 | 0.24 |
| C8A | 17 | −8.67 | 0.23 | −2.75 | 0.20 | −0.03 | 0.24 |
| N7A | 17 | −8.84 | 0.32 | −4.00 | 0.25 | 0.37 | 0.15 |
| C5A | 17 | −9.17 | 0.33 | −4.65 | 0.16 | −0.75 | 0.14 |
| C6A | 17 | −9.46 | 0.45 | −6.00 | 0.16 | −0.92 | 0.24 |
| N6A | 17 | −9.49 | 0.52 | −6.85 | 0.31 | 0.08 | 0.37 |
| N1A | 17 | −9.74 | 0.48 | −6.40 | 0.12 | −2.17 | 0.29 |
| C2A | 17 | −9.75 | 0.40 | −5.55 | 0.19 | −3.19 | 0.18 |
| N3A | 17 | −9.49 | 0.29 | −4.26 | 0.16 | −3.07 | 0.11 |
| C4A | 17 | −9.20 | 0.23 | −3.82 | 0.08 | −1.83 | 0.13 |
| O3 | 17 | −4.01 | 0.22 | 3.14 | 0.33 | −2.03 | 0.34 |
| PN | 17 | −2.81 | 0.17 | 3.31 | 0.22 | −2.96 | 0.33 |
| O1N | 17 | −2.32 | 0.49 | 4.39 | 0.63 | −2.89 | 0.71 |
| O2N | 17 | −3.16 | 0.47 | 3.27 | 0.61 | −4.13 | 0.54 |
| O5′N | 17 | −1.87 | 0.29 | 2.15 | 0.26 | −2.49 | 0.48 |
| C5′N | 17 | −1.92 | 0.27 | 0.87 | 0.27 | −2.66 | 0.46 |
| C4′N | 17 | −0.83 | 0.19 | 0.02 | 0.24 | −2.14 | 0.36 |
| O4′N | 17 | 0.32 | 0.21 | 0.20 | 0.36 | −2.95 | 0.27 |
| C3′N | 17 | −0.36 | 0.23 | 0.40 | 0.28 | −0.74 | 0.32 |
| O3′N | 17 | −0.18 | 0.47 | −0.71 | 0.40 | 0.01 | 0.35 |
| C2′N | 17 | 0.91 | 0.23 | 1.05 | 0.40 | −0.94 | 0.21 |
| O2′N | 17 | 1.65 | 0.44 | 0.84 | 0.85 | 0.08 | 0.32 |
| C1′N | 17 | 1.45 | 0.18 | 0.41 | 0.23 | −2.17 | 0.22 |
| N1N | 17 | 2.44 | 0.15 | 1.17 | 0.24 | −2.89 | 0.19 |
| C2N | 17 | 3.61 | 0.20 | 0.61 | 0.24 | −3.24 | 0.16 |
| C3N | 17 | 4.53 | 0.22 | 1.30 | 0.35 | −3.97 | 0.23 |
| C7N | 17 | 5.81 | 0.29 | 0.71 | 0.58 | −4.39 | 0.38 |
| O7N | 17 | 6.57 | 0.47 | 1.16 | 0.94 | −4.83 | 0.51 |
| N7N | 17 | 6.03 | 0.44 | −0.27 | 0.96 | −4.27 | 0.71 |
| C4N | 17 | 4.30 | 0.34 | 2.55 | 0.41 | −4.33 | 0.47 |
| C5N | 17 | 3.12 | 0.39 | 3.09 | 0.48 | −3.96 | 0.64 |
| C6N | 17 | 2.19 | 0.27 | 2.41 | 0.44 | −3.24 | 0.51 |
| P2′ | 2 | −11.69 | 0.02 | 1.32 | 0.36 | −1.90 | 0.73 |
| OP1 | 2 | −12.69 | 0.51 | 0.79 | 0.45 | −1.31 | 1.66 |
| OP2 | 2 | −12.01 | 0.86 | 1.94 | 0.08 | −3.01 | 0.74 |
| OP3 | 2 | −11.04 | 0.61 | 2.17 | 0.59 | −1.12 | 0.07 |

TABLE 3D
Polypeptide and Solvent Interactors

| | residue- | | | | | | | | |
|---|---|---|---|---|---|---|---|---|---|
| atom name | mol. # | residue # | total | x | σx | y | σy | z | σz |
| Acceptors | | | | | | | | | |
| O | ALA 1 | 215 | | −4.41 | | −1.37 | | −4.378 | |
| O | VAL 2 | 268 | | −3.415 | | −1.508 | | −4.259 | |
| O | CYS 4 | 95 | | −3.525 | | −1.391 | | −4.201 | |
| O | VAL 5 | 392 | | −4.035 | | −1.223 | | −4.42 | |
| O | VAL 6 | 86 | | −2.622 | | −2.525 | | −3.463 | |
| O | VAL 7 | 268 | | −3.739 | | −1.583 | | −4.801 | |
| O | THR 8 | 274 | | −3.374 | | −1.505 | | −3.621 | |
| O | SER 9 | 76 | | −3.338 | | −0.96 | | −4.215 | |
| O | ALA10 | 237 | | −4.168 | | −1.334 | | −4.262 | |
| O | ALA11 | 242 | | −3.642 | | −1.13 | | −4.963 | |
| O | THR12 | 97 | | −2.827 | | −1.527 | | −3.709 | |
| O | PHE13 | 79 | | −3.279 | | −1.095 | | −4.527 | |
| O | VAL14 | 86 | | −2.698 | | −2.451 | | −3.496 | |
| O | THR15 | 96 | | −3.708 | | −1.231 | | −4.403 | |
| O | ASN17 | 254 | | −3.847 | | −1.386 | | −4.942 | |
| A15 | ACC | 15 | 15 | −3.508 | 0.51867 | −1.481 | 0.444684 | −4.244 | 0.48666 |
| O | CYS 1 | 236 | | 3.015 | | −2.169 | | −3.644 | |
| O | VAL 2 | 292 | | 3.319 | | −2.239 | | −3.966 | |
| O | THR 3 | 232 | | 3.626 | | −2.073 | | −5.277 | |
| O | ALA 4 | 136 | | 2.873 | | −1.964 | | −3.884 | |
| O | LEU 5 | 419 | | 3.566 | | −2.603 | | −2.54 | |
| O | VAL 6 | 128 | | 2.902 | | −2.638 | | −3.394 | |
| O | VAL 7 | 292 | | 3.435 | | −2.183 | | −4.536 | |
| O | ILE 8 | 298 | | 2.705 | | −2.013 | | −5.149 | |
| O | ILE 9 | 117 | | 3.267 | | −2.016 | | −3.572 | |
| O | VAL10 | 266 | | 3.531 | | −1.908 | | −3.445 | |
| O | VAL11 | 265 | | 2.245 | | −2.153 | | −5.774 | |
| O | VAL12 | 138 | | 3.423 | | −2.49 | | −3.658 | |
| O | GLY13 | 102 | | 3.045 | | −2.197 | | −3.332 | |
| O | VAL14 | 128 | | 2.473 | | −2.343 | | −3.403 | |
| O | ILE15 | 141 | | 3.095 | | −2.691 | | −3.316 | |
| O | ALA16 | 238 | | 3.132 | | −1.372 | | −5.812 | |
| O | THR17 | 282 | | 3.668 | | −1.893 | | −5.571 | |
| A22 | ACC | 22 | 17 | 3.1365 | 0.40729 | −2.173 | 0.325811 | −4.134 | 1.01093 |
| OG1 | THR 1 | 279 | | 6.933 | | 1.937 | | −8.332 | |
| O | ALA 3 | 297 | | 7.27 | | 2.615 | | −9.402 | |
| OD1 | ASN 8 | 345 | | 7.341 | | 0.057 | | −7.801 | |
| SG | CYS11 | 295 | | 8.12 | | 2.802 | | −8.368 | |
| OG | SER17 | 334 | | 7.164 | | 1.343 | | −7.29 | |
| A32 | ACC | 32 | 5 | 7.3656 | 0.44907 | 1.7508 | 1.109256 | −8.239 | 0.78586 |
| SG | CYS 2 | 46 | | 1.759 | | 6.095 | | −1.597 | |
| OG | SER 6 | 240 | | 1.154 | | 5.714 | | −0.415 | |
| SG | CYS 7 | 46 | | 1.39 | | 6.091 | | −1.637 | |
| OD1 | ASN 8 | 190 | | 1.47 | | 6.205 | | −3.174 | |
| OG | SER 9 | 222 | | 0.831 | | 6.625 | | −0.409 | |
| OG | SER10 | 133 | | 0.616 | | 5.761 | | −3.752 | |
| A34 | ACC | 34 | 6 | 1.2033 | 0.42444 | 6.0818 | 0.331268 | −1.831 | 1.38661 |
| OD1 | ASP 2 | 223 | | −12.06 | | −1.364 | | −3.72 | |
| OD1 | ASP 3 | 175 | | −12.31 | | −1.116 | | −2.892 | |
| OD1 | ASP 4 | 52 | | −12.29 | | −1.122 | | −4.018 | |
| OD2 | ASP 6 | 41 | | −12.14 | | −1.461 | | −3.317 | |
| OD2 | ASP 7 | 223 | | −12.26 | | 0.192 | | −5.072 | |
| OE1 | GLU 8 | 242 | | −12.17 | | −0.604 | | −3.687 | |
| OD1 | ASP 9 | 34 | | −11.26 | | −2.188 | | −3.753 | |
| OD2 | ASP10 | 197 | | −12.39 | | −1.306 | | −3.358 | |
| OD1 | ASP12 | 53 | | −11.79 | | −1.526 | | −3.647 | |
| OE1 | GLU14 | 41 | | −11.76 | | −1.641 | | −3.303 | |
| OD1 | ASP15 | 53 | | −11.95 | | −1.38 | | −3.606 | |
| OD1 | ASP16 | 181 | | −12.33 | | −1.128 | | −3.23 | |
| OD1 | ASP17 | 221 | | −11.74 | | −1.235 | | −3.585 | |
| A47 | ACC | 47 | 13 | −12.03 | 0.32497 | −1.221 | 0.556926 | −3.63 | 0.51984 |
| OD2 | ASP 2 | 223 | | −10.46 | | −0.712 | | −5.067 | |
| OD2 | ASP 3 | 175 | | −10.78 | | −0.582 | | −4.327 | |
| OD2 | ASP 4 | 52 | | −10.23 | | −0.845 | | −4.641 | |
| OD1 | ASP 6 | 41 | | −10.8 | | −0.87 | | −4.98 | |
| OD1 | ASP 7 | 223 | | −10.78 | | −1.36 | | −4.58 | |
| OE2 | GLU 8 | 242 | | −10.46 | | 0.103 | | −4.803 | |
| OD2 | ASP 9 | 34 | | −9.97 | | −1.147 | | −5.144 | |
| OD1 | ASP10 | 197 | | −10.71 | | −0.756 | | −4.609 | |
| OD2 | ASP12 | 53 | | −10.1 | | −0.987 | | −4.85 | |
| OE1 | GLU13 | 38 | | −11.44 | | −1.444 | | −4.68 | |
| OE2 | GLU14 | 41 | | −10.7 | | −0.348 | | −4.708 | |
| OD2 | ASP15 | 53 | | −10.49 | | −0.813 | | −5.102 | |
| OD2 | ASP16 | 181 | | −10.87 | | −0.595 | | −4.761 | |
| OD2 | ASP17 | 221 | | −10.38 | | −0.678 | | −5.134 | |
| A48 | ACC | 48 | 14 | −10.58 | 0.37106 | −0.788 | 0.394449 | −4.813 | 0.24544 |
| O | ILE 2 | 269 | | −2.445 | | −2.256 | | −0.193 | |
| O | VAL 3 | 205 | | −2.446 | | −3.051 | | −1.43 | |
| O | ALA 4 | 96 | | −3.129 | | −3.442 | | −1.462 | |
| OG | SER 6 | 88 | | −2.227 | | −3.432 | | −0.657 | |
| O | ILE 7 | 269 | | −2.544 | | −2.277 | | −0.546 | |
| O | ALA 9 | 77 | | −2.936 | | −3.387 | | −1.405 | |
| O | VAL10 | 238 | | −2.653 | | −2.624 | | −0.587 | |
| O | ALA12 | 98 | | −3.101 | | −4.038 | | −1.238 | |
| O | THR13 | 80 | | −2.808 | | −2.299 | | −1.065 | |
| O | LEU15 | 97 | | −2.726 | | −2.902 | | −1.459 | |
| O | VAL16 | 211 | | −2.296 | | −2.734 | | −1.354 | |
| A53 | ACC | 53 | 11 | −2.665 | 0.30695 | −2.949 | 0.580767 | −1.036 | 0.45723 |
| O | ALA 2 | 317 | | 7.471 | | −2.554 | | −6.143 | |
| OD2 | ASP 3 | 258 | | 8.172 | | −2.402 | | −6.366 | |
| OG | SER 4 | 161 | | 7.049 | | −2.744 | | −6.487 | |
| O | LEU 6 | 154 | | 8.715 | | −2.807 | | −5.528 | |
| O | CYS 7 | 317 | | 7.229 | | −2.526 | | −6.12 | |
| O | VAL 9 | 146 | | 7.764 | | −1.709 | | −6.821 | |
| OG | SER12 | 163 | | 6.66 | | −2.956 | | −6.767 | |
| O | MET14 | 154 | | 8.194 | | −2.694 | | −5.797 | |
| OG1 | THR15 | 166 | | 6.339 | | −2.915 | | −6.856 | |
| OD2 | ASP16 | 264 | | 8.236 | | −1.758 | | −6.216 | |
| OD1 | ASP17 | 308 | | 7.288 | | −2.414 | | −6.878 | |
| A57 | ACC | 57 | 11 | 7.5561 | 0.73228 | −2.498 | 0.420521 | −6.362 | 0.45202 |
| ND1 | HIS 4 | 193 | | 10.626 | | 0.61 | | −3.116 | |
| ND1 | HIS 6 | 186 | | 10.014 | | −0.093 | | −2.576 | |
| ND1 | HIS 9 | 177 | | 10.504 | | 1.695 | | −3.436 | |
| ND1 | HIS12 | 195 | | 10.555 | | 0.375 | | −3.145 | |
| ND1 | HIS14 | 186 | | 9.53 | | 0.058 | | −2.803 | |
| ND1 | HIS15 | 198 | | 10.182 | | 0.378 | | −2.754 | |
| A96 | ACC | 96 | 6 | 10.235 | 0.41864 | 0.5038 | 0.635226 | −2.972 | 0.31587 |
| O | THR 4 | 247 | | 1.697 | | 6.212 | | −4.932 | |
| O | SER 6 | 241 | | 1.512 | | 5.836 | | −4.992 | |
| O | THR12 | 246 | | 1.401 | | 6.459 | | −5.282 | |
| O | THR15 | 248 | | 1.165 | | 6.252 | | −5.758 | |
| A99 | ACC | 99 | 4 | 1.4438 | 0.22235 | 6.1898 | 0.25949 | −5.241 | 0.37703 |
| Donors | | | | | | | | | |
| N | SER 1 | 174 | | −6.971 | | 2.982 | | −6.833 | |
| N | GLY 2 | 201 | | −7.051 | | 2.265 | | −6.475 | |
| N | GLY 3 | 154 | | −8.12 | | 2.219 | | −6.064 | |
| N | GLY 4 | 29 | | −7.293 | | 1.675 | | −6.476 | |
| N | GLY 5 | 313 | | −7.132 | | 2.483 | | −6.314 | |
| N | GLY 6 | 13 | | −8.808 | | 2.734 | | −6.39 | |
| N | GLY 7 | 201 | | −7.089 | | 2.378 | | −6.44 | |
| N | GLY 8 | 221 | | −7.171 | | 2.192 | | −6.095 | |
| N | GLY 9 | 10 | | −8.673 | | 2.272 | | −6.033 | |
| N | GLY10 | 176 | | −7.708 | | 1.61 | | −6.214 | |
| N | GLY11 | 176 | | −7.166 | | 2.546 | | −5.844 | |
| N | GLY12 | 30 | | −7.358 | | 1.997 | | −6.529 | |
| N | GLY13 | 15 | | −8.347 | | 3.129 | | −5.659 | |
| N | GLY14 | 13 | | −8.993 | | 2.681 | | −6.03 | |
| N | GLY15 | 30 | | −7.35 | | 1.898 | | −6.417 | |
| N | GLY16 | 160 | | −7.754 | | 2.152 | | −6.234 | |
| N | GLY17 | 200 | | −7.84 | | 1.819 | | −6.562 | |
| D9 | DON | 9 | 17 | −7.696 | 0.66531 | 2.296 | 0.431519 | −6.271 | 0.29226 |
| OG | SER 1 | 174 | | −4.169 | | 3.811 | | −6 | |
| N | GLY 2 | 202 | | −5.086 | | 5.296 | | −6.262 | |
| N | HIS 3 | 155 | | −6.067 | | 5.154 | | −2.788 | |
| N | PHE 4 | 30 | | −5.313 | | 4.474 | | −6.084 | |
| N | GLU 5 | 314 | | −5.224 | | 5.566 | | −5.679 | |
| N | GLN 6 | 14 | | −6.138 | | 5.075 | | −5.705 | |
| N | GLY 7 | 202 | | −5.115 | | 5.35 | | −5.842 | |
| N | ASP 8 | 222 | | −4.822 | | 4.792 | | −5.908 | |
| N | GLY 9 | 11 | | −6.29 | | 5.058 | | −5.51 | |
| N | VAL10 | 177 | | −5.677 | | 4.573 | | −6.103 | |
| N | PRO11 | 177 | | −5.131 | | 5.547 | | −5.772 | |
| N | ALA12 | 31 | | −5.256 | | 4.982 | | −5.907 | |
| N | ARG13 | 16 | | −5.501 | | 5.429 | | −5.154 | |
| N | GLN14 | 14 | | −6.311 | | 5.136 | | −5.537 | |
| N | ASN15 | 31 | | −5.383 | | 4.826 | | −5.877 | |
| N | HIS16 | 161 | | −5.882 | | 5.126 | | −5.388 | |
| N | ARG17 | 201 | | −6 | | 4.758 | | −5.866 | |
| D10 | DON | 10 | 17 | −5.492 | 0.57597 | 4.9972 | 0.439163 | −5.787 | 0.2765 |
| N | VAL 1 | 177 | | −2.231 | | 4.172 | | −8.191 | |
| N | VAL 2 | 203 | | −2.521 | | 4.333 | | −7.106 | |
| N | ILE 3 | 156 | | −3.616 | | 4.356 | | −7.328 | |
| N | VAL 4 | 31 | | −2.539 | | 3.702 | | −7.072 | |
| N | ALA 5 | 315 | | −2.542 | | 4.593 | | −6.385 | |
| N | ILE 6 | 15 | | −3.471 | | 4.432 | | −7.048 | |
| N | VAL 7 | 203 | | −2.643 | | 4.75 | | −6.934 | |
| N | VAL 8 | 223 | | −2.523 | | 3.344 | | −6.862 | |
| N | ILE 9 | 12 | | −3.863 | | 4.694 | | −6.846 | |
| N | VAL10 | 178 | | −3.08 | | 3.512 | | −7.145 | |
| N | VAL11 | 178 | | −2.953 | | 4.368 | | −7.142 | |
| N | VAL12 | 32 | | −2.793 | | 3.892 | | −6.902 | |
| N | MET13 | 17 | | −3.251 | | 4.443 | | −6.48 | |
| N | ILE14 | 15 | | −3.826 | | 4.526 | | −7.009 | |
| N | VAL15 | 32 | | −2.951 | | 3.934 | | −7.082 | |
| N | ILE16 | 162 | | −3.722 | | 4.618 | | −7.096 | |
| N | ILE17 | 202 | | −3.556 | | 4.064 | | −7.229 | |
| D12 | DON | 12 | 17 | −3.064 | 0.53062 | 4.2196 | 0.418148 | −7.05 | 0.38051 |
| OG1 | THR 1 | 279 | | 6.933 | | 1.937 | | −8.332 | |
| OG | SER17 | 334 | | 7.164 | | 1.343 | | −7.29 | |
| D34 | DON | 34 | 2 | 7.0485 | 0.16334 | 1.64 | 0.420021 | −7.811 | 0.73681 |
| SG | CYS 2 | 46 | | 1.759 | | 6.095 | | −1.597 | |
| OG | SER 6 | 240 | | 1.154 | | 5.714 | | −0.415 | |
| SG | CYS 7 | 46 | | 1.39 | | 6.091 | | −1.637 | |
| OG | SER 9 | 222 | | 0.831 | | 6.625 | | −0.409 | |
| D36 | DON | 36 | 4 | 1.2835 | 0.39114 | 6.1313 | 0.374531 | −1.015 | 0.6959 |
| ND2 | ASN 2 | 225 | | −14.56 | | 3.056 | | −1.923 | |
| ND2 | ASN 7 | 225 | | −15.12 | | 3.202 | | −1.587 | |
| ND2 | ASN10 | 199 | | −14.92 | | 2.944 | | −1.285 | |
| N | ARG11 | 200 | | −15.34 | | 3.078 | | −2.669 | |
| ND2 | ASN15 | 55 | | −14.92 | | 2.794 | | −2.271 | |
| D53 | DON | 53 | 5 | −14.97 | 0.2886 | 3.0148 | 0.153705 | −1.947 | 0.54651 |
| N | VAL 2 | 294 | | 2.334 | | −2.69 | | −0.397 | |
| N | ASN 4 | 138 | | 2.277 | | −2.379 | | 0.029 | |
| N | ASN 5 | 421 | | 2.644 | | −2.578 | | 0.583 | |
| N | ASN 6 | 130 | | 2.063 | | −2.785 | | −0.349 | |
| N | VAL 7 | 294 | | 2.742 | | −3.152 | | −1.066 | |
| N | ASN 9 | 119 | | 2.504 | | −2.09 | | −0.346 | |
| N | VAL10 | 268 | | 4.124 | | −4.101 | | −1.602 | |
| N | ASN12 | 140 | | 2.522 | | −2.522 | | −0.359 | |
| N | THR13 | 104 | | 2.237 | | −3.331 | | 0.05 | |
| N | ASN14 | 130 | | 1.53 | | −2.648 | | −0.196 | |
| N | ASN15 | 143 | | 2.106 | | −2.7 | | −0.15 | |
| D61 | DON | 61 | 11 | 2.4621 | 0.64303 | −2.816 | 0.543046 | −0.346 | 0.5762 |
| NH1 | ARG 3 | 234 | | 4.587 | | −0.618 | | 0.683 | |
| ND2 | ASN 4 | 138 | | 5.58 | | −1.025 | | −0.579 | |
| ND2 | ASN 5 | 421 | | 4.967 | | −0.91 | | −0.857 | |
| ND2 | ASN 6 | 130 | | 4.796 | | 0.498 | | −0.376 | |
| ND2 | ASN 9 | 119 | | 4.776 | | 1.072 | | −0.333 | |
| ND2 | ASN12 | 140 | | 4.874 | | 0.88 | | −0.41 | |
| ND2 | ASN14 | 130 | | 3.87 | | 0.241 | | −0.144 | |
| ND2 | ASN15 | 143 | | 4.582 | | 0.661 | | −0.159 | |
| NH1 | ARG16 | 240 | | 5.381 | | −0.809 | | −0.472 | |
| NH2 | ARG16 | 240 | | 4.57 | | 1.118 | | 0.462 | |
| NH1 | ARG17 | 284 | | 4.55 | | −1.163 | | −0.589 | |
| D84 | DON | 84 | 11 | 4.7757 | 0.4524 | −0.005 | 0.904651 | −0.252 | 0.45674 |
| ND1 | HIS 4 | 193 | | 10.626 | | 0.61 | | −3.116 | |
| ND1 | HIS 6 | 186 | | 10.014 | | −0.093 | | −2.576 | |
| ND1 | HIS 9 | 177 | | 10.504 | | 1.695 | | −3.436 | |
| N | ASN10 | 299 | | 10.126 | | 0.746 | | −3.889 | |
| ND1 | HIS12 | 195 | | 10.555 | | 0.375 | | −3.145 | |
| ND1 | HIS14 | 186 | | 9.53 | | 0.058 | | −2.803 | |
| ND1 | HIS15 | 198 | | 10.182 | | 0.378 | | −2.754 | |
| D105 | DON | 105 | 7 | 10.22 | 0.38439 | 0.5384 | 0.587058 | −3.103 | 0.45095 |
| NE | ARG 9 | 80 | | −3.463 | | 6.961 | | −1.445 | |
| NH1 | ARG12 | 101 | | −3.963 | | 7.113 | | −1.977 | |
| NE | ARG13 | 16 | | −3.284 | | 7.146 | | −1.239 | |
| NE2 | GLN14 | 14 | | −5.2 | | 6.85 | | −1.788 | |
| D148 | DON | 148 | 4 | −3.978 | 0.86417 | 7.0175 | 0.137697 | −1.612 | 0.33227 |
| Waters | | | | | | | | | |
| O | HOH 1 | 37 | | −4.852 | | 0.916 | | −5.955 | |
| O | HOH 2 | 6 | | −4.639 | | 1.155 | | −5.586 | |
| O | HOH 3 | 341 | | −5.542 | | 1.121 | | −5.837 | |
| O | HOH 4 | 4 | | −4.423 | | 0.776 | | −5.661 | |
| O | HOH 5 | 8 | | −4.893 | | 1.328 | | −5.536 | |
| O | HOH 6 | 58 | | −4.815 | | 1.672 | | −6.392 | |
| O | HOH 9 | 316 | | −5.086 | | 1.405 | | −5.627 | |
| O | HOH10 | 3 | | −4.816 | | 0.793 | | −5.596 | |
| O | HOH12 | 21 | | −4.532 | | 0.966 | | −5.406 | |
| O | HOH13 | 810 | | 4.598 | | 2.049 | | −5.765 | |
| O | HOH14 | 20 | | −5.549 | | 1.612 | | −6.137 | |
| O | HOH15 | 370 | | −4.601 | | 1.061 | | −5.784 | |
| O | HOH16 | 566 | | −4.928 | | 1.656 | | −6.021 | |
| O | HOH17 | 35 | | −5.091 | | 1.06 | | −5.977 | |
| W1 | WAT | 1 | 14 | −4.883 | 0.34302 | 1.255 | 0.378799 | −5.806 | 0.26779 |
| O | HOH 1 | 238 | | −11.09 | | 4.575 | | −3.702 | |
| O | HOH 4 | 62 | | −10.9 | | 3.609 | | −3.539 | |
| O | HOH 6 | 71 | | −10.22 | | 3.569 | | −2.078 | |
| O | HOH10 | 92 | | −11.17 | | 3.592 | | −2.43 | |
| O | HOH15 | 395 | | −10.54 | | 3.897 | | −3.702 | |
| O | HOH17 | 199 | | −11.04 | | 3.484 | | −3.197 | |
| W6 | WAT | 6 | 6 | −10.83 | 0.3724 | 3.7877 | 0.410386 | −3.108 | 0.69569 |
| O | HOH 3 | 360 | | −12.48 | | 2.562 | | −5.14 | |
| O | HOH 5 | 495 | | −12.31 | | 1.96 | | −5.591 | |
| O | HOH17 | 439 | | −12.49 | | 2.145 | | −5.979 | |
| W19 | WAT | 19 | 3 | −12.43 | 0.09854 | 2.2223 | 0.308361 | −5.57 | 0.41989 |

TABLE 4A
Pharmacofamily 2 Subset

| | | | rmsd from |
|---|---|---|---|
| | | | Family |
| molecule # | pdb | type | Avg. |
| 1 | 1CH6 | Glutamine Dehydrogenase (cow) | 0.58 |
| 2 | 1CER | Glyceraldehyde-3-phosphate D. | |
| | | (Thermus aquaticus) | 0.31 |
| 3 | 1GYP | Glyceraldehyde-3-phosphate D. | 0.34 |
| | | (Leishmania Mexicana) | 0.33 |
| 4 | 2HDH | L3-hydroxyacyl CoA D. (human) | |
| 5 | 1BXG | Phenylalanine D. (Rhodococcus sp.) | 0.59 |

TABLE 4B
Polypeptide and Solvent Interactors (average coordinates)

| atom | residue- | | | | | | | |
|---|---|---|---|---|---|---|---|---|
| name | mol.# | total | x | σx | y | σy | z | σz |
| Acceptors | | | | | | | | |
| A4 | ACC | 1 | 1.10 | — | −4.12 | — | 7.02 | — |
| A21 | ACC | 5 | −7.31 | 0.94 | 7.30 | 0.23 | 1.70 | 0.42 |
| A24 (D28) | ACC | 2 | −9.52 | 0.99 | 4.80 | 0.06 | −0.72 | 0.16 |
| A26 | ACC | 3 | −0.46 | 0.40 | 0.62 | 0.26 | 1.22 | 0.20 |
| A31 | ACC | 5 | 5.50 | 0.30 | 1.15 | 0.72 | 4.41 | 0.31 |
| A36 | ACC | 4 | 8.61 | 0.66 | −1.12 | 0.22 | 6.56 | 0.54 |
| A45 | ACC | 2 | −5.73 | 0.51 | 5.08 | 0.20 | −7.62 | 0.21 |
| A47 | ACC | 2 | −2.38 | 0.16 | 1.11 | 0.32 | 1.01 | 0.14 |
| A57 | ACC | 3 | 4.82 | 0.39 | 1.19 | 0.27 | 12.29 | 0.39 |
| A74 | ACC | 1 | 1.86 | — | −2.87 | — | 1.92 | — |
| A75 | ACC | 1 | 3.26 | — | −4.52 | — | 2.27 | — |
| A80 | ACC | 1 | 5.45 | — | −2.88 | — | 6.60 | — |
| Donors | | | | | | | | |
| D21 | DON | 5 | −3.69 | 0.38 | 6.81 | 0.18 | 5.90 | 0.25 |
| D22 | DON | 6 | −2.46 | 0.68 | 4.98 | 0.17 | 8.91 | 0.34 |
| D24 | DON | 3 | 0.28 | 0.18 | 4.88 | 0.18 | 8.67 | 0.22 |
| D27 | DON | 5 | −8.64 | 0.42 | 7.78 | 0.77 | −0.88 | 0.39 |
| D28 (A24) | DON | 3 | −9.48 | 0.70 | 4.58 | 0.39 | −0.74 | 0.11 |
| D37 | DON | 2 | 4.89 | 0.32 | −0.97 | 0.08 | 1.99 | 0.02 |
| D38 | DON | 2 | 5.09 | 0.86 | −3.25 | 0.34 | 4.18 | 0.69 |
| D84 | DON | 1 | −10.79 | — | 7.18 | — | 0.38 | — |
| Water | | | | | | | | |
| W1 | WAT | 2 | −1.68 | 0.35 | 5.44 | 0.29 | 5.49 | 0.17 |

TABLE 4C
NAD(P) Conformer Model

| atom name | total | x | σx | y | σy | z | σz |
|---|---|---|---|---|---|---|---|
| PA | 5 | −4.24 | 0.19 | 1.80 | 0.11 | 6.48 | 0.23 |
| O1A | 5 | −5.08 | 0.52 | 0.75 | 0.25 | 6.07 | 0.45 |
| O2A | 5 | −4.62 | 0.23 | 2.55 | 0.14 | 7.71 | 0.23 |
| O5′A | 5 | −3.99 | 0.30 | 2.86 | 0.25 | 5.34 | 0.17 |
| C5′A | 5 | −4.32 | 0.41 | 2.73 | 0.18 | 4.00 | 0.21 |
| C4′A | 5 | −4.89 | 0.25 | 4.02 | 0.13 | 3.50 | 0.21 |
| O4′A | 5 | −4.66 | 0.06 | 4.05 | 0.14 | 2.08 | 0.25 |
| C3′A | 5 | −6.39 | 0.28 | 4.19 | 0.08 | 3.68 | 0.05 |
| O3′A | 5 | −6.70 | 0.35 | 5.46 | 0.12 | 4.28 | 0.08 |
| C2′A | 5 | −6.97 | 0.10 | 3.99 | 0.10 | 2.31 | 0.09 |
| O2′A | 5 | −8.13 | 0.10 | 4.75 | 0.15 | 2.08 | 0.23 |
| C1′A | 5 | −5.83 | 0.08 | 4.47 | 0.05 | 1.44 | 0.09 |
| N9A | 5 | −5.83 | 0.28 | 3.93 | 0.08 | 0.08 | 0.09 |
| C8A | 5 | −6.06 | 0.43 | 2.68 | 0.11 | −0.38 | 0.12 |
| N7A | 5 | −5.93 | 0.46 | 2.59 | 0.16 | −1.71 | 0.12 |
| C5A | 5 | −5.61 | 0.32 | 3.84 | 0.14 | −2.10 | 0.08 |
| C6A | 5 | −5.33 | 0.30 | 4.34 | 0.13 | −3.42 | 0.12 |
| N6A | 5 | −5.40 | 0.43 | 3.59 | 0.10 | −4.50 | 0.12 |
| N1A | 5 | −5.02 | 0.16 | 5.67 | 0.11 | −3.48 | 0.08 |
| C2A | 5 | −4.98 | 0.15 | 6.46 | 0.10 | −2.39 | 0.12 |
| N3A | 5 | −5.23 | 0.19 | 6.03 | 0.05 | −1.15 | 0.07 |
| C4A | 5 | −5.53 | 0.23 | 4.70 | 0.09 | −1.02 | 0.07 |
| O3 | 5 | −2.84 | 0.26 | 1.29 | 0.52 | 6.62 | 0.32 |
| PN | 5 | −1.40 | 0.20 | 1.34 | 0.15 | 7.08 | 0.12 |
| O1N | 5 | −1.38 | 0.09 | 0.38 | 0.31 | 7.92 | 0.81 |
| O2N | 5 | −1.08 | 0.38 | 2.54 | 0.62 | 7.45 | 0.53 |
| O5′N | 5 | −0.51 | 0.24 | 1.01 | 0.62 | 5.97 | 0.12 |
| C5′N | 5 | −0.17 | 0.26 | 1.53 | 0.19 | 4.90 | 0.36 |
| C4′N | 5 | 1.07 | 0.22 | 0.97 | 0.17 | 4.29 | 0.20 |
| O4′N | 5 | 2.15 | 0.28 | 1.09 | 0.07 | 5.24 | 0.14 |
| C3′N | 5 | 1.04 | 0.26 | −0.49 | 0.20 | 3.88 | 0.12 |
| O3′N | 5 | 1.75 | 0.42 | −0.71 | 0.28 | 2.70 | 0.12 |
| C2′N | 5 | 1.72 | 0.26 | −1.20 | 0.10 | 5.03 | 0.16 |
| O2′N | 5 | 2.24 | 0.33 | −2.42 | 0.17 | 4.63 | 0.40 |
| C1′N | 5 | 2.76 | 0.26 | −0.18 | 0.11 | 5.44 | 0.12 |
| NN1 | 2 | 3.11 | 0.26 | −0.28 | 0.02 | 6.85 | 0.14 |
| C2N | 5 | 2.34 | 0.16 | −0.31 | 0.27 | 7.90 | 0.13 |
| C3N | 5 | 2.82 | 0.09 | −0.46 | 0.18 | 9.20 | 0.15 |
| C7N | 5 | 1.92 | 0.16 | −0.56 | 0.40 | 10.40 | 0.11 |
| O7N | 5 | 2.01 | 0.59 | −0.69 | 0.67 | 11.28 | 0.54 |
| NN7 | 2 | 0.66 | 0.05 | −0.71 | 1.04 | 10.09 | 0.19 |
| C4N | 5 | 4.19 | 0.10 | −0.48 | 0.22 | 9.46 | 0.21 |
| C5N | 5 | 5.02 | 0.08 | −0.40 | 0.46 | 8.34 | 0.31 |
| C6N | 5 | 4.56 | 0.17 | −0.26 | 0.34 | 7.06 | 0.27 |

TABLE 4D
Polypeptide and Solvent Interactors

| | residue- | | | | | | | | |
|---|---|---|---|---|---|---|---|---|---|
| atom name | mol. # | residue # | total | x | σx | y | σy | z | σz |
| Acceptors | | | | | | | | | |
| OD1 | ASN 1 | 168 | | 1.095 | | -4.122 | | 7.015 | |
| A4 | ACC | 4 | 1 | 1.095 | | -4.122 | | 7.015 | |
| O | PHE 1 | 252 | | -5.191 | | 8.539 | | 6.797 | |
| O | PHE 2 | 8 | | -5.255 | | 8.065 | | 6.21 | |
| O | PHE 3 | 10 | | -4.805 | | 8.465 | | 5.853 | |
| O | GLY 4 | 23 | | -4.854 | | 8.511 | | 7.292 | |
| O | LEU 5 | 183 | | -5.255 | | 8.273 | | 6.6 | |
| A14 | ACC | 14 | 5 | -5.072 | 0.22358 | 8.3706 | 0.199937 | 6.5504 | 0.55124 |
| OE1 | GLU 1 | 275 | | -6.7 | | 7.256 | | 2.045 | |
| OD1 | ASP 2 | 32 | | -8.197 | | 7.417 | | 1.98 | |
| OD1 | ASP 3 | 38 | | -5.963 | | 7.483 | | 1.973 | |
| OD1 | ASP 4 | 45 | | -7.792 | | 7.445 | | 1.259 | |
| OD1 | ASP 5 | 205 | | -7.896 | | 6.916 | | 1.22 | |
| A21 | ACC | 21 | 5 | -7.31 | 0.94194 | 7.3034 | 0.233204 | 1.6954 | 0.41735 |
| OG | SER 1 | 276 | | -10.22 | | 4.761 | | -0.611 | |
| OG1 | THR 5 | 206 | | -8.824 | | 4.845 | | -0.836 | |
| A24 | ACC | 24 | 2 | -9.523 | 0.98783 | 4.803 | 0.059397 | -0.724 | 0.1591 |
| O | ALA 1 | 326 | | -0.312 | | 0.409 | | 1.158 | |
| O | ILE 4 | 108 | | -0.908 | | 0.539 | | 1.439 | |
| O | ALA 5 | 239 | | -0.153 | | 0.904 | | 1.064 | |
| A26 | ACC | 26 | 3 | -0.458 | 0.39802 | 0.6173 | 0.256629 | 1.2203 | 0.19512 |
| O | GLY 1 | 347 | | 5.243 | | 2.256 | | 4.521 | |
| O | THR 2 | 119 | | 5.496 | | 1.074 | | 4.297 | |
| O | SER 3 | 134 | | 5.492 | | 0.484 | | 4.132 | |
| O | ASN 4 | 135 | | 5.99 | | 0.551 | | 4.206 | |
| O | ALA 5 | 260 | | 5.254 | | 1.362 | | 4.897 | |
| A31 | ACC | 31 | 5 | 5.495 | 0.30275 | 1.1454 | 0.720452 | 4.4106 | 0.30869 |
| OD1 | ASN 1 | 374 | | 9.186 | | -0.987 | | 5.966 | |
| NE2 | HIS 4 | 158 | | 7.894 | | -1.364 | | 7.028 | |
| OD1 | ASN 5 | 288 | | 8.756 | | -0.995 | | 6.691 | |
| A36 | ACC | 36 | 4 | 8.612 | 0.65793 | -1.115 | 0.215389 | 6.5617 | 0.54268 |
| O | LYS 2 | 77 | | -6.092 | | 4.938 | | -7.77 | |
| O | GLN 3 | 91 | | -5.369 | | 5.217 | | -7.467 | |
| A45 | ACC | 45 | 2 | -5.731 | 0.51124 | 5.0775 | 0.197283 | 7.619 | 0.21425 |
| O | THR 2 | 96 | | -2.488 | | 1.334 | | 0.905 | |
| O | THR 3 | 111 | | -2.265 | | 0.887 | | 1.109 | |
| A47 | ACC | 47 | 2 | -2.377 | 0.15768 | 1.1105 | 0.316077 | 1.007 | 0.14425 |
| O | GLY 2 | 97 | | -0.425 | | -2.183 | | -0.802 | |
| O | GLY 3 | 112 | | -0.663 | | -2.629 | | -0.591 | |
| O | VAL 4 | 109 | | -1.565 | | -1.362 | | -0.563 | |
| A49 | ACC | 49 | 3 | -0.884 | 0.60137 | 2.058 | 0.642683 | -0.652 | 0.13066 |
| O | ASN 2 | 313 | | 4.587 | | 0.929 | | 12.609 | |
| O | ASN 3 | 335 | | 5.271 | | 1.175 | | 12.408 | |
| OG1 | THR 5 | 153 | | 4.596 | | 1.474 | | 11.8759 | |
| A57 | ACC | 57 | 3 | 4.818 | 0.39234 | 1.1927 | 0.272929 | 12.292 | 0.38822 |
| OE1 | GLU 4 | 110 | | 1.86 | | -2.87 | | 1.915 | |
| A74 | ACC | 74 | 1 | 1.86 | | -2.87 | | 1.915 | |
| OE2 | GLU 4 | 110 | | 3.257 | | -4.521 | | 2.267 | |
| A75 | ACC | 75 | 1 | 3.257 | | -4.521 | | 2.267 | |
| OG | SER 4 | 137 | | 5.445 | | -2.882 | | 6.6 | |
| A80 | ACC | 80 | 1 | 5.445 | | -2.882 | | 6.6 | |
| Donors | | | | | | | | | |
| N | PHE 1 | 252 | | -3.795 | | 8.382 | | 3.66 | |
| N | PHE 2 | 8 | | -3.513 | | 8.186 | | 3.399 | |
| N | PHE 3 | 10 | | -3.274 | | 8.183 | | 2.802 | |
| N | GLY 4 | 23 | | -3.891 | | 8.194 | | 3.841 | |
| N | LEU 5 | 183 | | -3.951 | | 8.196 | | 3.424 | |
| D20 | DON | 20 | 5 | -3.685 | 0.28452 | 8.2282 | 0.086146 | 3.4252 | 0.39277 |
| N | GLY 1 | 253 | | -3.608 | | 7.062 | | 6.079 | |
| N | GLY 2 | 9 | | -3.411 | | 6.805 | | 5.974 | |
| N | GLY 3 | 11 | | -3.279 | | .847 | | 5.562 | |
| N | GLY 4 | 24 | | -3.951 | | 6.79 | | 6.145 | |
| N | GLY 5 | 184 | | -4.182 | | 6.562 | | 5.718 | |
| D21 | DON | 21 | 5 | -3.686 | 0.37537 | 6.8132 | 0.17801 | 5.8956 | 0.24739 |
| N | ASN 1 | 254 | | -2.527 | | 5.077 | | 8.825 | |
| N | ARG 2 | 10 | | -2.87 | | 4.723 | | 8.75 | |
| N | ARG 3 | 12 | | -2.609 | | 4.907 | | 8.456 | |
| N | LEU 4 | 25 | | -3 | | 5.05 | | 9.249 | |
| N | VAL 5 | 186 | | -1.3 | | 5.165 | | 9.257 | |
| D22 | DON | 22 | 6 | -2.461 | 0.67675 | 4.9844 | 0.173072 | 8.9074 | 0.34432 |
| N | VAL 1 | 255 | | 0.427 | | 5.067 | | 8.691 | |
| N | ILE 2 | 11 | | 0.083 | | 4.702 | | 8.883 | |
| N | ILE 3 | 13 | | 0.32 | | 4.862 | | 8.448 | |
| D24 | DON | 24 | 3 | 0.2767 | 0.17605 | 4.877 | 0.182962 | 8.674 | 0.218 |
| N | SER 1 | 276 | | -8.021 | | 9.758 | | -1.068 | |
| N | LEU 2 | 33 | | -8.808 | | 8.195 | | -0.527 | |
| N | MET 3 | 39 | | -9.137 | | 8.038 | | -0.417 | |
| N | GLN 4 | 46 | | -8.461 | | 9.672 | | -1.048 | |
| N | THR 5 | 206 | | -8.757 | | 7.228 | | -1.324 | |
| D27 | DON | 27 | 5 | -8.637 | 0.41955 | 7.7782 | 0.77195 | -0.877 | 0.38718 |
| OG | SER 1 | 276 | | -10.22 | | 4.761 | | -0.611 | |
| NE2 | GLN 4 | 46 | | -9.404 | | 4.137 | | -0.763 | |
| OG1 | THR 5 | 206 | | -8.824 | | 4.845 | | -0.836 | |
| D28 | DON | 28 | 3 | -9.483 | 0.70184 | 4.581 | 0.386802 | -0.737 | 0.11479 |
| N | ASN 1 | 349 | | 4.665 | | -0.919 | | 1.972 | |
| N | ASNS | 262 | | 5.113 | | -1.03 | | 1.998 | |
| D37 | DON | 37 | 2 | 4.889 | 0.31678 | -0.975 | 0.078489 | 1.985 | 0.01838 |
| ND2 | ASN 1 | 349 | | 4.485 | | -3.489 | | 4.665 | |
| N | SER4 | 137 | | 5.697 | | -3.011 | | 3.686 | |
| D38 | DON | 38 | 2 | 5.091 | 0.85701 | -3.25 | 0.337997 | 4.1755 | 0.69226 |
| N | ASP 5 | 207 | | -10.79 | | 7.181 | | 0.384 | |
| D84 | DON | 84 | 1 | -10.79 | | 7.181 | | 0.384 | |
| Waters | | | | | | | | | |
| O | HOH 4 | 888 | | -1.436 | | 5.238 | | 5.606 | |
| O | HOH 5 | 888 | | -1.931 | | 5.647 | | 5.365 | |
| W1 | WAT | 1 | 1 | -1.684 | 0.35002 | 5.4425 | 0.289207 | 5.4855 | 0.17041 |

TABLE 5A
Pharmacofamily 3 Subset

| | | | RMSD from |
|---|---|---|---|
| Molecule # | pdb | type | Family Avg. |
| 1 | 1A27 | 17b-Hydroxysteroid | 0.35 |
| | | Dehydrogenase (human) | |
| 2 | 1AE1 | Tropinone Reductase | 0.33 |
| 3 | 1AHH | 7a-Hydroxysteroid Dehydrogenase | 0.51 |
| 4 | 1BDB | Cis-Biphenyl-2,3-Dihydrodiol- | 0.28 |
| | | 2,3-Dehydrogenase | |
| 5 | 1BSV | GDP-Fucose Synthase | 0.87 |
| 6 | 1CYD | Carbonyl Reductase | 0.26 |
| 7 | 1ENZ | Enoyl Acyl Carrier Protein | 0.66 |
| | | Reductase | |
| 8 | 1NAI | UDP-Galactose Epimerase | 0.45 |
| 9 | 1SEP | Sepiapterin Reductase | 0.43 |
| 10 | 1YBV | Trihydroxynaphthalene Reductase | 0.70 |
| 11 | 1HSD | 2a-20b-Hydroxysteroid | 0.55 |
| | | Dehydrogenase | |
| 12 | 1DIR | Dihydropteridine Reductase | 0.75 |

TABLE 5B
Polypeptide and Solvent Interactors (average coordinates)

| atom name | Name | total | x | σx | y | σy | z | σz |
|---|---|---|---|---|---|---|---|---|
| Acceptors | | | | | | | | |
| A5 (D5) | ACC | 4 | −9.243 | 0.6136 | −6.385 | 0.485759 | 7.5835 | 0.60521 |
| A20 | ACC | 10 | −2.055 | 0.62558 | −12.31 | 0.344913 | 15.347 | 0.71676 |
| A24 | ACC | 12 | −0.54 | 0.89267 | −1.809 | 0.373379 | 8.7658 | 0.6637 |
| A32 | ACC | 12 | 2.8272 | 0.30273 | 5.1573 | 0.670541 | 10.018 | 0.502 |
| A34 (D34) | ACC | 9 | 1.8439 | 0.50418 | 7.7642 | 0.274322 | 13.139 | 0.30794 |
| A36 (D38) | ACC | 12 | −0.113 | 0.24453 | 4.7021 | 0.586493 | 13.952 | 0.24008 |
| A38 | ACC | 11 | 1.2485 | 0.72569 | 9.7629 | 0.441462 | 9.482 | 0.48385 |
| A40 | ACC | 10 | −2.496 | 0.41035 | 10.064 | 0.558296 | 8.9034 | 0.77733 |
| A42 | ACC | 9 | −7.86 | 0.22197 | 8.1173 | 0.560664 | 9.1394 | 0.53745 |
| A44 (D47) | ACC | 8 | −8.336 | 0.72492 | 4.1414 | 0.50819 | 9.0466 | 0.81437 |
| A68 | ACC | 5 | −6.27 | 0.3454 | −7.233 | 0.556879 | 7.5474 | 0.30836 |
| Donors | | | | | | | | |
| D5 (A5) | DON | 6 | −9.892 | 1.12248 | −6.493 | 0.603878 | 7.9562 | 0.75319 |
| D7 | DON | 2 | −9.66 | 0.00919 | −1.843 | 0.165463 | 8.0065 | 0.15061 |
| D9 | DON | 12 | −6.057 | 0.41875 | 1.6692 | 0.293883 | 4.914 | 0.25367 |
| D21 | DON | 10 | 0.0467 | 0.43511 | −11.62 | 0.342553 | 11.981 | 0.91633 |
| D34 (A34) | DON | 9 | 1.8439 | 0.50418 | 7.7642 | 0.274322 | 13.139 | 0.30794 |
| D38 (A36) | DON | 11 | −0.113 | 0.24453 | 4.7021 | 0.586493 | 13.952 | 0.24008 |
| D40 | DON | 12 | 2.4988 | 0.36354 | 1.5327 | 0.445563 | 12.367 | 0.3007 |
| D45 | DON | 10 | −5.476 | 0.54512 | 9.6232 | 0.478163 | 8.6938 | 0.41629 |
| D47 (A44) | DON | 6 | −7.675 | 0.22275 | 3.8897 | 0.368935 | 9.5875 | 1.11949 |
| Water | | | | | | | | |
| W4 | WAT | 9 | −4.738 | 0.3561 | −1.037 | 0.298174 | 6.477 | 0.47268 |
| W5 | WAT | 4 | 2.6995 | 0.66749 | −0.925 | 0.394841 | 9.7795 | 0.39679 |
| W9 | WAT | 9 | 3.273 | 0.73202 | −1.012 | 0.573841 | 12.802 | 0.86657 |
| W11 | WAT | 6 | −6.007 | 0.19132 | −1.829 | 0.200188 | 13.702 | 0.2296 |

TABLE 5C
NAD(P) Conformer Model

| atom | | | | | | | |
|---|---|---|---|---|---|---|---|
| name | total | x | σx | y | σy | z | σz |
| PA | 12 | −6.94 | 0.27682 | −0.359 | 0.12062 | 10.196 | 0.3132 |
| O1A | 12 | −7.187 | 0.50362 | −0.724 | 0.311997 | 11.568 | 0.35149 |
| O2A | 12 | −8.039 | 0.23033 | 0.0836 | 0.236246 | 9.4105 | 0.49965 |
| O5′A | 12 | −6.324 | 0.33618 | −1.599 | 0.152174 | 9.5178 | 0.48615 |
| C5′A | 12 | −5.31 | 0.27378 | −2.37 | 0.252109 | 9.8483 | 0.42032 |
| C4′A | 12 | −5.39 | 0.23487 | −3.716 | 0.196458 | 9.4463 | 0.27041 |
| O4′A | 12 | −4.443 | 0.17889 | −4.486 | 0.362347 | 10.152 | 0.45942 |
| C3′A | 12 | −6.677 | 0.26263 | −4.369 | 0.172555 | 9.6349 | 0.38881 |
| O3′A | 12 | −7.077 | 0.60241 | −4.969 | 0.317672 | 8.502 | 0.51095 |
| C2′A | 12 | −6.427 | 0.2192 | −5.392 | 0.18758 | 10.719 | 0.34471 |
| O2′A | 12 | −7.207 | 0.43164 | −6.53 | 0.229629 | 10.538 | 0.52325 |
| C1′A | 12 | −4.996 | 0.2692 | −5.707 | 0.273621 | 10.514 | 0.28506 |
| N9A | 12 | −4.338 | 0.16157 | −6.335 | 0.231445 | 11.625 | 0.21234 |
| C8A | 12 | −4.321 | 0.18366 | −5.957 | 0.287413 | 12.906 | 0.25525 |
| N7A | 12 | −3.708 | 0.19062 | −6.853 | 0.38173 | 13.663 | 0.14123 |
| C5A | 12 | −3.345 | 0.167 | −7.802 | 0.336217 | 12.81 | 0.08303 |
| C6A | 12 | −2.685 | 0.29854 | −8.972 | 0.409416 | 13.085 | 0.20366 |
| N6A | 12 | −2.353 | 0.40839 | −9.302 | 0.557888 | 14.313 | 0.25603 |
| N1A | 12 | −2.439 | 0.38208 | −9.778 | 0.395034 | 12.051 | 0.30817 |
| C2A | 12 | −2.826 | 0.38939 | −9.443 | 0.393263 | 10.824 | 0.25264 |
| N3A | 12 | −3.468 | 0.30202 | −8.33 | 0.362823 | 10.533 | 0.10763 |
| C4A | 12 | −3.726 | 0.15519 | −7.514 | 0.288774 | 11.545 | 0.09427 |
| O3 | 12 | −5.803 | 0.3398 | 0.7197 | 0.195007 | 10.133 | 0.2437 |
| PN | 12 | −5.139 | 0.15801 | 1.6654 | 0.119922 | 9.0683 | 0.30355 |
| O1N | 12 | −5.513 | 0.30736 | 2.837 | 0.583522 | 9.2767 | 0.62893 |
| O2N | 12 | −5.465 | 0.24079 | 1.3618 | 0.579089 | 7.8578 | 0.57479 |
| O5′N | 12 | −3.623 | 0.17622 | 1.5297 | 0.454033 | 9.3583 | 0.46312 |
| C5′N | 12 | −2.693 | 0.23195 | 0.8583 | 0.262204 | 8.7345 | 0.42939 |
| C4′N | 12 | −1.318 | 0.21148 | 1.311 | 0.296942 | 9.1289 | 0.3066 |
| O4′N | 12 | −1.218 | 0.20704 | 2.7193 | 0.281646 | 8.9326 | 0.16566 |
| C3′N | 12 | −1.013 | 0.32386 | 1.0723 | 0.442515 | 10.567 | 0.32728 |
| O3′N | 12 | 0.2498 | 0.44917 | 0.5617 | 0.307845 | 10.743 | 0.48253 |
| C2′N | 12 | −1.071 | 0.433 | 2.4089 | 0.415664 | 11.195 | 0.2308 |
| O2′N | 12 | −0.264 | 0.66117 | 2.4258 | 0.295043 | 12.27 | 0.42485 |
| C1′N | 12 | −0.686 | 0.16367 | 3.3148 | 0.345237 | 10.0094 | 0.21704 |
| N1N | 12 | −1.199 | 0.0741 | 4.663 | 0.296089 | 10.265 | 0.17649 |
| C2N | 12 | −2.555 | 0.09392 | 4.903 | 0.192059 | 10.257 | 0.12994 |
| C3N | 12 | −3.045 | 0.15342 | 6.1843 | 0.177656 | 10.413 | 0.22204 |
| C7N | 12 | −4.492 | 0.16456 | 6.5182 | 0.22133 | 10.516 | 0.29939 |
| O7N | 12 | −4.912 | 0.2416 | 7.4728 | 0.677128 | 10.793 | 0.41339 |
| N7N | 12 | −5.319 | 0.24693 | 5.7468 | 0.705835 | 10.295 | 0.42085 |
| C4N | 12 | −2.139 | 0.24246 | 7.2165 | 0.188473 | 10.586 | 0.22472 |
| C5N | 12 | −0.79 | 0.23943 | 6.9686 | 0.319535 | 10.576 | 0.31698 |
| C6N | 12 | −0.303 | 0.12398 | 5.6903 | 0.375214 | 10.42 | 0.30569 |
| P2′ | 6 | −8.185 | 0.35266 | −7.167 | 0.53148 | 11.087 | 0.59086 |
| OP1 | 6 | −8.864 | 0.54615 | −7.461 | 1.469844 | 10.462 | 0.97819 |
| OP2 | 6 | −8.7 | 0.98419 | −7.192 | 1.218849 | 11.053 | 0.61709 |
| OP3 | 6 | −7.909 | 0.42562 | −7.322 | 0.715581 | 12.334 | 0.66989 |

TABLE 5D
Polypeptide and Solvent Interactors

| | residue- | | | | | | | | |
|---|---|---|---|---|---|---|---|---|---|
| atom name | mol. # | residue # | total | x | σx | y | σy | z | σz |
| Acceptors | | | | | | | | | |
| O | GLY 1 | 9 | | −4.643 | | −4.27 | | 6.043 | |
| O | GLY 2 | 28 | | −4.558 | | −4.117 | | 5.821 | |
| O | GLY 3 | 18 | | −4.048 | | −4.273 | | 6.088 | |
| O | GLY 4 | 12 | | −4.135 | | −3.933 | | 6.033 | |
| O | GLY 5 | 10 | | −4.432 | | −4.169 | | 5.555 | |
| O | GLY 6 | 14 | | −4.284 | | −4.355 | | 6.044 | |
| O | GLY 7 | 14 | | −6.249 | | −5.065 | | 6.52 | |
| O | GLY 8 | 7 | | −4.849 | | −3.848 | | 5.762 | |
| O | GLY 9 | 15 | | −4.591 | | −3.878 | | 5.357 | |
| O | GLY10 | 36 | | −4.346 | | −4.384 | | 5.754 | |
| O | GLY11 | 13 | | −5.058 | | −4.026 | | 6.159 | |
| O | GLY12 | 13 | | −5.622 | | −4.826 | | 5.87 | |
| A1 | ACC | 1 | 12 | −4.735 | 0.64211 | −4.262 | 0.369162 | 5.9172 | 0.30204 |
| OG | SER 1 | 11 | | −9.556 | | −5.885 | | 8.172 | |
| OG | SER 2 | 30 | | −9.127 | | −6.766 | | 7.066 | |
| OG | SER 8 | 36 | | −9.85 | | −6.053 | | 8.039 | |
| OG | SER 9 | 17 | | −8.437 | | −6.835 | | 7.057 | |
| A5 | ACC | 5 | 4 | −9.243 | 0.6136 | −6.385 | 0.485759 | 7.5835 | 0.60521 |
| OD1 | ASP 1 | 65 | | −1.811 | | −12.31 | | 14.284 | |
| OD1 | ASP 2 | 78 | | −2.629 | | −12.15 | | 15.593 | |
| OD2 | ASP 3 | 68 | | −1.583 | | −12.75 | | 16.533 | |
| OD2 | ASP 4 | 59 | | −2.534 | | −12.5 | | 15.835 | |
| OD1 | ASP 6 | 60 | | −2.109 | | −11.85 | | 15.924 | |
| OD1 | ASP 7 | 64 | | −2.151 | | −12.8 | | 14.21 | |
| OD2 | ASP 8 | 58 | | −2.841 | | −11.82 | | 15.085 | |
| OD1 | ASP 9 | 70 | | −2.628 | | −12.13 | | 15.425 | |
| OD1 | ASN 10 | 87 | | −1.218 | | −12.17 | | 15.492 | |
| OD1 | ASP11 | 60 | | −1.044 | | −12.57 | | 15.088 | |
| A20 | ACC | 20 | 10 | −2.055 | 0.62558 | −12.31 | 0.344913 | 15.347 | 0.71676 |
| O | ASN 1 | 90 | | −0.231 | | −1.804 | | 8.763 | |
| O | ASN 2 | 106 | | −0.349 | | −1.37 | | 8.814 | |
| O | ASN 3 | 95 | | 0.522 | | −1.353 | | 8.638 | |
| O | ASN 4 | 86 | | 0.101 | | −1.425 | | 8.863 | |
| O | ALA 5 | 62 | | −1.699 | | −2.266 | | 8.014 | |
| O | ASN 6 | 83 | | −0.206 | | −1.697 | | 9.086 | |
| O | ALA 7 | 94 | | −2.052 | | −2.486 | | 7.753 | |
| O | PHE 8 | 80 | | −1.247 | | −1.892 | | 9.217 | |
| O | ASN 9 | 101 | | −0.131 | | −1.62 | | 8.833 | |
| O | ASN10 | 114 | | 0.159 | | −1.576 | | 9.032 | |
| O | ASN11 | 87 | | −0.643 | | −1.744 | | 9.231 | |
| O | VAL12 | 82 | | −2.283 | | −1.889 | | 7.62 | |
| A24 | ACC | 24 | 12 | −0.672 | 0.92482 | −1.76 | 0.344669 | 8.6553 | 0.5546 |
| O | GLY 1 | 141 | | 2.663 | | 5.67 | | 8.586 | |
| O | SER 2 | 157 | | 2.57 | | 5.524 | | 10.215 | |
| O | THR 3 | 145 | | 2.691 | | 4.785 | | 10.423 | |
| O | ILE 4 | 141 | | 3.141 | | 4.744 | | 10.048 | |
| O | GLY 5 | 106 | | 2.669 | | 4.9 | | 10.086 | |
| O | SER 6 | 135 | | 2.664 | | 4.979 | | 10.231 | |
| O | ASP 7 | 148 | | 2.413 | | 6.773 | | 9.962 | |
| O | SER 8 | 123 | | 3.033 | | 5.584 | | 9.704 | |
| O | SER 9 | 157 | | 2.652 | | 5.344 | | 10.012 | |
| O | GLYlO | 163 | | 3.026 | | 4.753 | | 10.51 | |
| O | SERil | 138 | | 2.901 | | 4.576 | | 10.07 | |
| O | GLY12 | 132 | | 3.503 | | 4.256 | | 10.366 | |
| A32 | ACC | 32 | 12 | 2.8272 | 0.30273 | 5.1573 | 0.670541 | 10.018 | 0.502 |
| OG | SER 1 | 142 | | 1.908 | | 7.501 | | 12.689 | |
| OG | SER 2 | 158 | | 1.217 | | 8.135 | | 13.294 | |
| OG | SER 3 | 146 | | 1.984 | | 7.724 | | 13.283 | |
| OG | SER 4 | 142 | | 2.278 | | 7.462 | | 12.615 | |
| OG | SER 5 | 107 | | 1.06 | | 7.551 | | 13.088 | |
| OG | SER 8 | 124 | | 2.726 | | 8.12 | | 13.565 | |
| OG | SER 9 | 158 | | 1.901 | | 8.072 | | 13.351 | |
| OG | SERlO | 164 | | 1.664 | | 7.735 | | 13.227 | |
| OG | SERil | 139 | | 1.857 | | 7.578 | | 13.136 | |
| A34 | ACC | 34 | 9 | 1.8439 | 0.50418 | 7.7642 | 0.274322 | 13.139 | 0.30794 |
| OH | TYR 1 | 155 | | −0.171 | | 5.291 | | 14.251 | |
| OH | TYR 2 | 171 | | −0.291 | | 4.635 | | 13.936 | |
| OH | TYR 3 | 159 | | 0.016 | | 5.509 | | 14.332 | |
| OH | TYR 4 | 155 | | 0.03 | | 4.468 | | 13.891 | |
| OH | TYR 5 | 136 | | −0.098 | | 3.379 | | 13.966 | |
| OH | TYR 6 | 149 | | −0.376 | | 4.379 | | 13.778 | |
| OH | TYR 8 | 149 | | 0.166 | | 4.681 | | 13.768 | |
| OH | TYR 9 | 171 | | −0.28 | | 4.756 | | 13.633 | |
| OH | TYR10 | 178 | | −0.441 | | 4.469 | | 14.27 | |
| OH | TYR11 | 152 | | −0.176 | | 4.772 | | 13.685 | |
| OH | TYR12 | 146 | | 0.376 | | 5.384 | | 13.961 | |
| A36 | ACC | 36 | 12 | −0.113 | 0.24453 | 4.7021 | 0.586493 | 13.952 | 0.24008 |
| O | CYS 1 | 185 | | 1.067 | | 9.484 | | 9.076 | |
| O | PRO 2 | 201 | | 0.576 | | 10.012 | | 9.398 | |
| O | PRO 3 | 189 | | 0.411 | | 9.713 | | 9.099 | |
| O | SER 4 | 184 | | 1.319 | | 9.083 | | 8.553 | |
| O | PRO 5 | 163 | | 2.198 | | 10.158 | | 9.311 | |
| O | PRO 6 | 179 | | 0.756 | | 9.916 | | 10.316 | |
| O | ALA 7 | 191 | | 0.898 | | 10.562 | | 9.433 | |
| O | TYR8 | 177 | | 1.702 | | 10.131 | | 9.844 | |
| O | PRO10 | 208 | | 1.679 | | 9.684 | | 9.536 | |
| O | PRO11 | 182 | | 0.511 | | 9.318 | | 9.88 | |
| O | PRO12 | 178 | | 2.617 | | 9.331 | | 9.856 | |
| A38 | ACC | 38 | 11 | 1.2485 | 0.72569 | 9.7629 | 0.441462 | 9.482 | 0.48385 |
| O | GLY 1 | 186 | | −2.149 | | 9.494 | | 8.888 | |
| O | GLY 2 | 202 | | −2.874 | | 10.159 | | 9.066 | |
| O | GLY 3 | 190 | | −2.748 | | 9.972 | | 8.954 | |
| O | GLY 4 | 185 | | −2.235 | | 9.16 | | 8.272 | |
| O | THR 6 | 180 | | −2.406 | | 9.993 | | 9.592 | |
| O | GLY 7 | 192 | | −2.617 | | 10.505 | | 8.651 | |
| O | PHE 8 | 178 | | −1.769 | | 10.522 | | 10.103 | |
| O | GLY 9 | 200 | | −2.438 | | 9.522 | | 8.495 | |
| O | GLY11 | 183 | | −2.476 | | 10.303 | | 9.636 | |
| O | THR12 | 180 | | −3.248 | | 11.005 | | 7.377 | |
| A40 | ACC | 40 | 10 | −2.496 | 0.41035 | 10.064 | 0.558296 | 8.9034 | 0.77733 |
| O | VAL 1 | 188 | | −7.78 | | 7.375 | | 8.869 | |
| O | ILE 2 | 204 | | −8.015 | | 7.969 | | 8.848 | |
| O | ILE 3 | 192 | | −7.824 | | 8.024 | | 8.259 | |
| O | ILE 4 | 187 | | −8.021 | | 7.996 | | 9.727 | |
| O | VAL 6 | 182 | | −7.651 | | 7.627 | | 9.43 | |
| O | ILE 7 | 194 | | −7.928 | | 8.273 | | 9.726 | |
| O | LEU 9 | 202 | | −8.114 | | 8.807 | | 9.429 | |
| O | ILE10 | 211 | | −7.407 | | 7.823 | | 8.498 | |
| O | THR11 | 185 | | −7.996 | | 9.162 | | 9.469 | |
| A42 | ACC | 42 | 9 | −7.86 | 0.22197 | 8.1173 | 0.560664 | 9.1394 | 0.53745 |
| OG1 | THR 1 | 190 | | −7.639 | | 3.969 | | 9.24 | |
| OG1 | THR 3 | 194 | | −8.9 | | 4.567 | | 8.706 | |
| OG | SER 4 | 189 | | −7.82 | | 3.618 | | 10.069 | |
| OG1 | THR 6 | 184 | | −7.838 | | 4.124 | | 9.427 | |
| OG1 | THR 7 | 196 | | −8.489 | | 3.692 | | 7.941 | |
| OD1 | ASN 9 | 204 | | −8.271 | | 5.097 | | 10.004 | |
| OG1 | THR10 | 213 | | −7.925 | | 4.335 | | 9.016 | |
| OG1 | THR11 | 187 | | −9.807 | | 3.729 | | 7.97 | |
| A44 | ACC | 44 | 8 | −8.336 | 0.72492 | 4.1414 | 0.508189 | 9.0466 | 0.81437 |
| OD2 | ASP 3 | 42 | | −6.103 | | −7.068 | | 7.363 | |
| OD2 | ASP 4 | 36 | | −5.98 | | −7.048 | | 7.173 | |
| OG1 | THR 6 | 38 | | −6.172 | | −8.219 | | 7.479 | |
| OD2 | ASP11 | 37 | | −6.23 | | −6.97 | | 7.91 | |
| OD2 | ASP12 | 37 | | −6.865 | | −6.862 | | 7.812 | |
| A68 | ACC | 68 | 5 | −6.27 | 0.3454 | −7.233 | 0.556879 | 7.5474 | 0.30836 |
| Donors | | | | | | | | | |
| OG | SER 1 | 11 | | −9.556 | | −5.885 | | 8.172 | |
| OG | SER 2 | 30 | | −9.127 | | −6.766 | | 7.066 | |
| NE | ARG 4 | 41 | | −11.43 | | −6.012 | | 8.513 | |
| OG | SER 8 | 36 | | −9.85 | | −6.053 | | 8.039 | |
| OG | SER 9 | 17 | | −8.437 | | −6.835 | | 7.057 | |
| OG | SER10 | 63 | | −10.95 | | −7.408 | | 8.89 | |
| D5 | DON | 5 | 6 | −9.892 | 1.12248 | −6.493 | 0.603878 | 7.9562 | 0.75319 |
| N | SER 1 | 12 | | −9.161 | | −3.738 | | 5.795 | |
| N | LYS 2 | 31 | | −9.063 | | −3.703 | | 5.456 | |
| N | ALA 3 | 21 | | −8.29 | | −4.331 | | 5.081 | |
| N | SER 4 | 15 | | −8.15 | | −3.721 | | 5.342 | |
| N | GLY 5 | 13 | | −7.45 | | −3.226 | | 6.074 | |
| N | LYS 6 | 17 | | −8.395 | | −4.321 | | 5.731 | |
| N | ILE 7 | 16 | | −9.025 | | −4.226 | | 5.612 | |
| N | GLY 8 | 10 | | −7.76 | | −3.367 | | 5.536 | |
| N | ARG 9 | 18 | | −8.859 | | −3.975 | | 5.692 | |
| N | ARG10 | 39 | | −8.674 | | −4.044 | | 4.836 | |
| N | ARG11 | 16 | | −8.652 | | −3.889 | | 5.427 | |
| N | GLY12 | 16 | | −8.476 | | −3.851 | | 6.412 | |
| D6 | DON | 6 | 12 | −8.496 | 0.5257 | −3.866 | 0.346377 | 5.5828 | 0.41764 |
| OG | SER 1 | 12 | | −9.666 | | −1.96 | | 8.113 | |
| OG | SER 4 | 15 | | −9.653 | | −1.726 | | 7.9 | |
| D7 | DON | 7 | 2 | −9.66 | 0.00919 | −1.843 | 0.165463 | 8.0065 | 0.15061 |
| N | GLY 1 | 13 | | −8.789 | | −0.1 | | 5.426 | |
| N | GLY 2 | 32 | | −9.284 | | −0.05 | | 5.677 | |
| N | GLY 3 | 22 | | −8.761 | | −0.722 | | 5.167 | |
| N | GLY 4 | 16 | | −8.685 | | −0.121 | | 5.731 | |
| N | MET 5 | 14 | | −7.572 | | 0.427 | | 6.428 | |
| N | GLY 6 | 18 | | −8.768 | | −0.685 | | 5.543 | |
| N | SER 7 | 20 | | −9.948 | | 1.364 | | 5.27 | |
| N | TYR 8 | 11 | | −8.49 | | 0.13 | | 6.189 | |
| N | GLY 9 | 19 | | −9.129 | | −0.325 | | 6.034 | |
| N | GLY10 | 40 | | −8.828 | | −0.408 | | 5.459 | |
| N | GLY11 | 17 | | −8.878 | | −0.198 | | 5.546 | |
| N | ALA12 | 17 | | −8.931 | | −0.155 | | 6.586 | |
| D8 | DON | 8 | 12 | −8.839 | 0.5466 | −0.07 | 0.552142 | 5.7547 | 0.45545 |
| N | ILE 1 | 14 | | −5.584 | | 1.406 | | 4.565 | |
| N | ILE 2 | 33 | | −6.262 | | 1.734 | | 5.106 | |
| N | ILE 3 | 23 | | −6.008 | | 1.568 | | 4.583 | |
| N | LEU 4 | 17 | | −5.882 | | 1.991 | | 5.224 | |
| N | VAL 5 | 15 | | −5.284 | | 1.794 | | 5.226 | |
| N | ILE 6 | 19 | | −5.843 | | 1.286 | | 4.804 | |
| N | ILE 7 | 21 | | −6.436 | | 2.018 | | 4.734 | |
| N | ILE 8 | 12 | | −6.417 | | 2.039 | | 4.837 | |
| N | PHE 9 | 20 | | −6.214 | | 1.631 | | 5.229 | |
| N | ILE10 | 41 | | −5.852 | | 1.601 | | 5.016 | |
| N | LEU11 | 18 | | −6.037 | | 1.845 | | 5.008 | |
| N | LEU12 | 18 | | −6.861 | | 1.117 | | 4.636 | |
| D9 | DON | 9 | 12 | −6.057 | 0.41875 | 1.6692 | 0.293883 | 4.914 | 0.25367 |
| N | LEU 1 | 36 | | −4.861 | | −11.14 | | 5.491 | |
| N | SER 2 | 52 | | −5.654 | | −10.93 | | 6.923 | |
| N | ASP 3 | 42 | | −4.048 | | −10.76 | | 6.515 | |
| N | ASP 4 | 36 | | −3.888 | | −11 | | 6.574 | |
| N | THR 6 | 38 | | −3.943 | | −10.92 | | 6.379 | |
| N | PHE 7 | 41 | | −6.508 | | −10.95 | | 7.546 | |
| N | ALA 9 | 42 | | −4.253 | | −10.74 | | 6.218 | |
| N | TYR10 | 60 | | −4.488 | | −11.11 | | 5.821 | |
| N | ASP11 | 37 | | −4.55 | | −10.8 | | 6.546 | |
| N | ASP12 | 37 | | −5.596 | | −11.16 | | 7.002 | |
| D11 | DON | 11 | 10 | −4.779 | 0.8737 | −10.95 | 0.15485 | 6.5015 | 0.58747 |
| N | VAL 1 | 66 | | 0.188 | | −11.57 | | 12.02 | |
| N | LEU 2 | 79 | | −0.75 | | −11.93 | | 12.873 | |
| N | ILE 3 | 69 | | 0.555 | | −10.96 | | 12.368 | |
| N | VAL 4 | 60 | | 0.173 | | −11.26 | | 12.105 | |
| N | LEU 6 | 61 | | −0.617 | | −11.88 | | 13.014 | |
| N | VAL 7 | 65 | | −0.2 | | −12.11 | | 11.698 | |
| N | ILE 8 | 59 | | 0.203 | | −11.54 | | 11.611 | |
| N | VAL10 | 88 | | 0.182 | | −11.52 | | 12.416 | |
| N | VAL11 | 61 | | 0.252 | | −11.53 | | 11.99 | |
| OH | TYR12 | 12 | | 0.481 | | −11.87 | | 9.718 | |
| D21 | DON | 21 | 10 | 0.0467 | 0.43511 | 11.62 | 0.342553 | 11.981 | 0.91633 |
| OG | SER 1 | 142 | | 1.908 | | 7.501 | | 12.689 | |
| OG | SER 2 | 158 | | 1.217 | | 8.135 | | 13.294 | |
| OG | SER 3 | 146 | | 1.984 | | 7.724 | | 13.283 | |
| OG | SER 4 | 142 | | 2.278 | | 7.462 | | 12.615 | |
| OG | SER 5 | 107 | | 1.06 | | 7.551 | | 13.088 | |
| OG | SER 8 | 124 | | 2.726 | | 8.12 | | 13.565 | |
| OG | SER 9 | 158 | | 1.901 | | 8.072 | | 13.351 | |
| OG | SER10 | 164 | | 1.664 | | 7.735 | | 13.227 | |
| OG | SER11 | 139 | | 1.857 | | 7.578 | | 13.136 | |
| D34 | DON | 34 | 9 | 1.8439 | 0.50418 | 7.7642 | 0.274322 | 13.139 | 0.30794 |
| OH | TYR 1 | 155 | | −0.171 | | 5.291 | | 14.251 | |
| OH | TYR 2 | 171 | | −0.291 | | 4.625 | | 13.936 | |
| OH | TYR 3 | 159 | | 0.016 | | 5.509 | | 14.332 | |
| OH | TYR 4 | 155 | | 0.03 | | 4.468 | | 13.891 | |
| OH | TYR 5 | 136 | | −0.098 | | 3.379 | | 13.966 | |
| OH | TYR 6 | 149 | | −0.376 | | 4.379 | | 13.788 | |
| OH | TYR 8 | 149 | | 0.166 | | 4.681 | | 13.768 | |
| OH | TYR 9 | 174 | | −0.28 | | 4.756 | | 13.633 | |
| OH | TYR10 | 178 | | −0.441 | | 4.469 | | 14.27 | |
| OH | TYR11 | 152 | | −0.176 | | 4.772 | | 13.685 | |
| OH | TYR12 | 146 | | 0.376 | | 5.384 | | 13.961 | |
| D38 | DON | 38 | 11 | 0.113 | 0.24453 | 4.7021 | 0.586493 | 13.952 | 0.24008 |
| NZ | LYS 1 | 159 | | 2.273 | | 1.347 | | 12.922 | |
| NZ | LYS 2 | 175 | | 2.774 | | 1.885 | | 12.501 | |
| NZ | LYS 3 | 163 | | 2.831 | | 1.966 | | 12.606 | |
| NZ | LYS 4 | 159 | | 2.945 | | 1.926 | | 11.968 | |
| NZ | LYS 5 | 140 | | 2.494 | | 0.716 | | 12.288 | |
| NZ | LYS 6 | 153 | | 2.639 | | 1.609 | | 12.544 | |
| NZ | LYS 7 | 165 | | 1.913 | | 2.31 | | 11.938 | |
| NZ | LYS 8 | 153 | | 2.821 | | 1.471 | | 12.018 | |
| NZ | LYS 9 | 175 | | 2.663 | | 1.484 | | 12.193 | |
| NZ | LYS10 | 182 | | 2.338 | | 1.274 | | 12.644 | |
| NZ | LYS11 | 156 | | 2.502 | | 1.768 | | 12.367 | |
| NZ | LYS12 | 150 | | 1.793 | | 0.996 | | 12.411 | |
| D40 | DON | 40 | 12 | 2.4988 | 0.36354 | 1.5627 | 0.445563 | 12.367 | 0.3007 |
| N | VAL 1 | 188 | | −5.575 | | 9.076 | | 8.69 | |
| N | ILE 2 | 204 | | −5.985 | | 9.861 | | 8.611 | |
| N | ILE 3 | 192 | | −5.491 | | 9.652 | | 7.982 | |
| N | ILE 4 | 187 | | −5.774 | | 9.173 | | 8.669 | |
| N | VAL 6 | 182 | | −5.726 | | 9.411 | | 9.22 | |
| N | TLE 7 | 194 | | −5.844 | | 10.081 | | 9.195 | |
| N | LEU 9 | 202 | | −5.489 | | 9.563 | | 8.577 | |
| N | ILE10 | 211 | | −5.165 | | 9.506 | | 8.351 | |
| N | THR11 | 185 | | −5.643 | | 10.664 | | 9.242 | |
| N | LEU12 | 181 | | −4.064 | | 9.245 | | 8.401 | |
| D45 | DON | 45 | 10 | −5.476 | 0.54512 | 9.6232 | 0.478163 | 8.6938 | 0.41629 |
| OG1 | THR 1 | 190 | | −7.639 | | 3.969 | | 9.24 | |
| OG | SER 4 | 189 | | −7.82 | | 3.618 | | 10.069 | |
| OG1 | THR 6 | 184 | | −7.838 | | 4.124 | | 9.427 | |
| NZ | LYS 8 | 84 | | −7.399 | | 3.308 | | 11.527 | |
| ND2 | ASN 9 | 204 | | −7.429 | | 3.984 | | 8.246 | |
| OG1 | THR10 | 213 | | −7.925 | | 4.335 | | 9.016 | |
| D47 | DON | 47 | 6 | −7.675 | 0.22275 | 3.8897 | 0.368935 | 9.5875 | 1.11949 |
| Water | | | | | | | | | |
| O | HOH 1 | 525 | | −4.833 | | −1.135 | | 6.451 | |
| O | HOH 2 | 46 | | −5.297 | | −1.061 | | 6.752 | |
| O | HOH 3 | 3 | | −4.845 | | −1.187 | | 6.502 | |
| O | HOH 4 | 516 | | −4.351 | | −0.821 | | 6.859 | |
| O | HOH 5 | 437 | | −4.101 | | −1.147 | | 6.704 | |
| O | HOH 6 | 10 | | −4.524 | | −1.331 | | 6.783 | |
| O | HOH 7 | 309 | | −4.955 | | −0.333 | | 5.377 | |
| O | HOH 8 | 2 | | −4.854 | | −1.09 | | 6.112 | |
| O | HOH 9 | 12 | | −4.878 | | −1.224 | | 6.753 | |
| W4 | WAT | 4 | 9 | −4.738 | 0.3561 | −1.037 | 0.298174 | 6.477 | 0.47268 |
| O | HOH 1 | 536 | | 3.343 | | −0.704 | | 9.644 | |
| O | HOH 5 | 429 | | 1.797 | | −0.842 | | 9.926 | |
| O | HOH 6 | 327 | | 3.022 | | −1.504 | | 10.239 | |
| O | HOH 7 | 293 | | 2.636 | | −0.648 | | 9.309 | |
| W5 | WAT | 5 | 4 | 2.6995 | 0.66749 | −0.925 | 0.394841 | 9.7795 | 0.39679 |
| O | HOH 1 | 556 | | 2.764 | | −1.43 | | 12.516 | |
| O | HOH 2 | 24 | | 3.482 | | −0.937 | | 11.868 | |
| O | HOH 3 | 72 | | 4.908 | | −0.703 | | 11.31 | |
| O | HOH 4 | 531 | | 3.597 | | −0.619 | | 12.808 | |
| O | HOH 5 | 433 | | 2.747 | | −2.319 | | 13.306 | |
| O | HOH 6 | 24 | | 3.505 | | −1.086 | | 12.854 | |
| O | HOH 7 | 292 | | 2.421 | | −0.63 | | 12.788 | |
| O | HOH 8 | 125 | | 2.922 | | −0.954 | | 13.552 | |
| O | HOH 9 | 6 | | 3.111 | | −0.428 | | 14.219 | |
| W9 | WAT | 9 | 9 | 3.273 | 0.73202 | −1.012 | 0.573841 | 12.802 | 0.86657 |
| O | HOH 1 | 573 | | −5.99 | | −1.752 | | 13.358 | |
| O | HOH 4 | 607 | | −6.095 | | −1.503 | | 13.507 | |
| O | HOH 5 | 484 | | −6.117 | | −1.942 | | 13.958 | |
| O | HOH 6 | 198 | | −6.206 | | −2.028 | | 13.818 | |
| O | HOH 8 | 31 | | −5.979 | | −1.748 | | 13.701 | |
| O | HOH 9 | 24 | | −5.657 | | −2 | | 13.87 | |
| W11 | WAT | 11 | 6 | −6.007 | 0.19132 | −1.829 | 0.200188 | 13.702 | 0.2296 |

TABLE 6A
Pharmacofamily 4 Subset

| | | | rmsd |
|---|---|---|---|
| | | | from |
| | | | family |
| molecule # | pdb | type | avg. |
| 1 | 2CAH | catalyse (Proteus Mirabilis) | 0.18 |
| 2 | 8CAT | catalyse (cow) | 0.18 |

TABLE 6B
Polypeptide and Solvent Interactors (average coordinates)

| | residue- | | | | | | | |
|---|---|---|---|---|---|---|---|---|
| atom name | mol. # | total | x | σx | y | σy | z | σz |
| Acceptors | | | | | | | | |
| A3 (D4) | ACC | 2 | −1.117 | 0.36133 | −3.964 | 0.13435 | −3.882 | 0.27082 |
| A6 (D7) | ACC | 2 | −10.03 | 0.10889 | −5.617 | 0.029698 | 1.223 | 0.1895 |
| A17 | ACC | 2 | 5.454 | 0.08697 | 2.473 | 0.195161 | −0.056 | 0.58973 |
| A19 (D30) | ACC | 2 | 3.405 | 0.48366 | 1.421 | 0.065761 | 4.934 | 0.05586 |
| A21 | ACC | 2 | 1.11 | 0.65478 | −7.271 | 0.181726 | −2.784 | 0.39527 |
| A35 | ACC | 2 | 3.372 | | −7.545 | | 0.205 | |
| Donors | | | | | | | | |
| D4 (A3) | DON | 2 | −1.117 | 0.36133 | −3.964 | 0.13435 | −3.882 | 0.27082 |
| D7 (A6) | DON | 2 | −10.03 | 0.10889 | −5.617 | 0.029698 | 1.223 | 0.1895 |
| D10 | DON | 2 | −6.918 | 0.49215 | −1.253 | 0.286378 | 7 | 0.28284 |
| D11 | DON | 2 | −6.419 | 0.19163 | 0.023 | 0.147078 | 5.184 | 0.18173 |
| D14 | DON | 2 | −6.153 | | 3.824 | | 6.584 | |
| D21 | DON | 2 | −2.402 | | 4.522 | | 6.578 | |
| D22 | DON | 2 | −2.704 | 0.0997 | 4.738 | 0.703571 | 9.015 | 0.19658 |
| D26 | DON | 2 | 4.609 | 0.02758 | 2.264 | 0.350018 | −2.894 | 0.51831 |
| D30 (A19) | DON | 2 | 3.405 | 0.48366 | 1.421 | 0.065761 | 4.934 | 0.05586 |
| D42 | DON | 2 | 3.907 | | 6.034 | | 0.45 | |
| Waters | | | | | | | | |
| W1 | WAT | 2 | 2.756 | | 3.789 | | −1.727 | |
| W3 | WAT | 2 | 7.572 | | −1.978 | | 4.115 | |

TABLE 6C
NAD(P) Conformer Model

| atom name | number | x | σx | y | σy | z | σz |
|---|---|---|---|---|---|---|---|
| PA | 2 | 2.91 | 0.04 | −2.21 | 0.03 | 5.65 | 0.05 |
| O1A | 2 | 2.72 | 0.06 | −3.30 | 0.15 | 6.64 | 0.05 |
| O2A | 2 | 3.84 | 0.02 | −1.14 | 0.13 | 6.03 | 0.21 |
| O5′A | 2 | 1.43 | 0.11 | −1.58 | 0.12 | 5.49 | 0.10 |
| C5′A | 2 | 0.37 | 0.04 | −2.46 | 0.22 | 4.99 | 0.04 |
| C4′A | 2 | −0.65 | 0.05 | −1.65 | 0.13 | 4.29 | 0.00 |
| O4′A | 2 | −1.84 | 0.18 | −2.41 | 0.04 | 4.08 | 0.03 |
| C3′A | 2 | −1.09 | 0.10 | −0.66 | 0.26 | 5.21 | 0.33 |
| O3′A | 2 | −0.77 | 0.41 | 0.64 | 0.09 | 5.13 | 0.06 |
| C2′A | 2 | −2.37 | 0.16 | −1.05 | 0.21 | 5.80 | 0.03 |
| O2′A | 2 | −3.24 | 0.42 | 0.04 | 0.54 | 6.17 | 0.19 |
| C1′A | 2 | −3.00 | 0.12 | −1.63 | 0.23 | 4.60 | 0.08 |
| N9A | 2 | −4.14 | 0.04 | −2.49 | 0.13 | 4.54 | 0.09 |
| C8A | 2 | −4.58 | 0.08 | −3.42 | 0.00 | 5.41 | 0.04 |
| N7A | 2 | −5.62 | 0.12 | −4.11 | 0.07 | 5.01 | 0.00 |
| C5A | 2 | −5.86 | 0.04 | −3.62 | 0.02 | 3.74 | 0.06 |
| C6A | 2 | −6.85 | 0.05 | −3.94 | 0.05 | 2.77 | 0.07 |
| N6A | 2 | −7.79 | 0.12 | −4.87 | 0.11 | 2.95 | 0.01 |
| N1A | 2 | −6.82 | 0.06 | −3.25 | 0.04 | 1.61 | 0.11 |
| C2A | 2 | −5.88 | 0.13 | −2.29 | 0.16 | 1.45 | 0.15 |
| N3A | 2 | −4.93 | 0.16 | −1.91 | 0.18 | 2.28 | 0.15 |
| C4A | 2 | −4.98 | 0.06 | −2.62 | 0.08 | 3.43 | 0.10 |
| O3 | 2 | 3.16 | 0.09 | −2.77 | 0.20 | 4.19 | 0.05 |
| PN | 2 | 4.13 | 0.03 | −2.43 | 0.03 | 3.00 | 0.01 |
| O1N | 2 | 5.29 | 0.18 | −3.36 | 0.17 | 3.00 | 0.07 |
| O2N | 2 | 4.47 | 0.33 | −1.02 | 0.09 | 2.89 | 0.03 |
| O5′N | 2 | 3.25 | 0.11 | −2.85 | 0.18 | 1.72 | 0.04 |
| C5′N | 2 | 2.89 | 0.14 | −4.22 | 0.12 | 1.54 | 0.19 |
| C4′N | 2 | 1.52 | 0.19 | −4.31 | 0.05 | 0.90 | 0.20 |
| O4′N | 2 | 0.53 | 0.15 | −3.57 | 0.13 | 1.66 | 0.23 |
| C3′N | 2 | 1.50 | 0.08 | −3.79 | 0.10 | −0.56 | 0.22 |
| O3′N | 2 | 1.58 | 0.07 | −4.98 | 0.12 | −1.40 | 0.15 |
| C2′N | 2 | 0.05 | 0.15 | −3.27 | 0.00 | −0.68 | 0.16 |
| O2′N | 2 | −0.79 | 0.07 | −4.25 | 0.19 | 1.31 | 0.32 |
| C1′N | 2 | −0.40 | 0.12 | −3.01 | 0.11 | 0.75 | 0.17 |
| N1N | 2 | −0.50 | 0.05 | −1.58 | 0.13 | 0.98 | 0.02 |
| C2N | 2 | 0.63 | 0.01 | −0.80 | 0.12 | 0.85 | 0.05 |
| C3N | 2 | 0.57 | 0.04 | 0.56 | 0.14 | 1.01 | 0.11 |
| C7N | 2 | 1.78 | 0.11 | 1.45 | 0.05 | 0.85 | 0.11 |
| C7N | 2 | 1.68 | 0.14 | 2.77 | 0.09 | 0.94 | 0.20 |
| N7N | 2 | 2.98 | 0.14 | 0.95 | 0.01 | 0.59 | 0.03 |
| C4N | 2 | −0.64 | 0.03 | 1.18 | 0.17 | 1.31 | 0.31 |
| C5N | 2 | −1.74 | 0.06 | 0.35 | 0.27 | 1.46 | 0.35 |
| C6N | 2 | −1.71 | 0.03 | −1.02 | 0.24 | 1.31 | 0.20 |
| P2′ | 2 | −3.70 | 0.19 | 0.63 | 0.15 | 7.56 | 0.08 |
| OP1 | 2 | −3.38 | 0.20 | −0.29 | 0.13 | 8.64 | 0.19 |
| OP2 | 2 | −5.04 | 0.42 | 1.06 | 0.50 | 7.59 | 0.15 |
| OP3 | 2 | −2.80 | 0.72 | 1.78 | 0.50 | 7.64 | 0.13 |

TABLE 6D
Polypeptide and Solvent Interactors

| | residue- | | | | | | | | |
|---|---|---|---|---|---|---|---|---|---|
| atom name | mol. # | residue # | total | x | σx | y | σy | z | σz |
| Acceptors | | | | | | | | | |
| NE2 | HIS 1 | 173 | | −1.37 | | −4.06 | | −3.69 | |
| NE2 | HIS 2 | 193 | | −0.86 | | −3.87 | | −4.07 | |
| A3 | ACC | 3 | 2 | −1.12 | 0.36 | −3.96 | 0.13 | −3.88 | 0.27 |
| OG | SER 1 | 180 | | −10.10 | | −5.60 | | 1.09 | |
| OG | SER 2 | 200 | | −9.95 | | −5.64 | | 1.36 | |
| A6 | ACC | 6 | 2 | −10.03 | 0.11 | −5.62 | 0.03 | 1.22 | 0.19 |
| O | TRP 1 | 282 | | 5.52 | | 2.34 | | −0.47 | |
| O | TRP 2 | 302 | | 5.39 | | 2.61 | | 0.36 | |
| A17 | ACC | 17 | 2 | 5.45 | 0.09 | 2.47 | 0.20 | −0.06 | 0.5 |
| ND1 | HIS 1 | 284 | | 3.06 | | 1.47 | | 4.970 | |
| ND1 | HIS 2 | 304 | | 3.75 | | 1.38 | | 4.89 | |
| A19 | ACC | 19 | 2 | 3.41 | 0.48 | 1.42 | 0.07 | 4.93 | 0.06 |
| O | GLN 1 | 421 | | 0.65 | | −7.40 | | −2.50 | |
| O | GLN 2 | 441 | | 1.57 | | −7.14 | | −3.06 | |
| A21 | ACC | 21 | 2 | 1.11 | 0.65 | −7.27 | 0.18 | −2.78 | 0.40 |
| OG1 | THR 2 | 444 | | 3.37 | | −7.55 | | 0.21 | |
| A35 | ACC | 35 | 2 | 3.37 | | −7.55 | | 0.21 | |
| Donors | | | | | | | | | |
| NE2 | HIS 1 | 173 | | −1.37 | | −4.06 | | −3.69 | |
| NE2 | HIS 2 | 193 | | −0.86 | | −3.87 | | −4.07 | |
| D4 | DON | 4 | 2 | −1.12 | 0.36 | −3.96 | 0.13 | −3.88 | 0.27 |
| OG | SER 1 | 180 | | −10.10 | | −5.60 | | −1.09 | |
| OG | SER 2 | 200 | | −9.95 | | −5.64 | | −1.36 | |
| D7 | DON | 7 | 2 | −10.03 | 0.11 | −5.62 | 0.03 | 1.22 | 0.19 |
| NH1 | ARG 1 | 182 | | −7.27 | | −1.05 | | 6.80 | |
| NH1 | ARG 2 | 202 | | −6.57 | | −1.46 | | 7.20 | |
| D10 | DON | 10 | 2 | −6.92 | 0.49 | −1.25 | 0.29 | 7.00 | 0.28 |
| NH2 | ARG 1 | 182 | | −6.28 | | 0.13 | | 5.06 | |
| NH2 | ARG 2 | 202 | | −6.56 | | −0.08 | | 5.31 | |
| D11 | DON | 11 | 2 | −6.42 | 0.19 | 0.02 | 0.15 | 5.18 | 0.18 |
| NE2 | HIS 1 | 192 | | −6.15 | | 3.82 | | 6.58 | |
| D14 | DON | 14 | 2 | −6.15 | | 3.82 | | 6.58 | |
| NH1 | ARG 1 | 216 | | −2.40 | | 4.52 | | 6.58 | |
| D21 | DON | 21 | 2 | −2.40 | | 4.52 | | 6.58 | |
| NH2 | ARG 1 | 216 | | −2.78 | | 4.24 | | 8.88 | |
| NZ | LYS 2 | 236 | | −2.63 | | 5.24 | | 9.15 | |
| D22 | DON | 22 | 2 | −2.70 | 0.10 | 4.74 | 0.70 | 9.02 | 0.20 |
| N | TRP 1 | 282 | | 4.59 | | 2.02 | | −3.26 | |
| N | TRP 2 | 302 | | 4.63 | | 2.51 | | −2.53 | |
| D26 | DON | 26 | 2 | 4.61 | 0.03 | 2.26 | 0.35 | −2.89 | 0.52 |
| ND1 | HIS 1 | 284 | | 3.06 | | 1.47 | | 4.97 | |
| ND1 | HIS 2 | 304 | | 3.75 | | 1.38 | | 4.89 | |
| D30 | DON | 30 | 2 | 3.41 | 0.48 | 1.42 | 0.07 | 4.93 | 0.06 |
| NE2 | GLN 2 | 281 | | 3.91 | | 6.03 | | 0.45 | |
| D42 | DON | 42 | 2 | 3.91 | | 6.03 | | 0.45 | |
| Waters | | | | | | | | | |
| O | HOH 1 | 10 | | 2.76 | | 3.79 | | −1.73 | |
| W1 | WAT | 1 | 2 | 2.7 | | 3.79 | | −1.73 | |
| O | HOH 1 | 12 | | 7.57 | | −1.98 | | 4.12 | |
| W3 | WAT | 3 | 2 | 7.57 | | −1.98 | | 4.12 | |

TABLE 7A
Pharmacofamily 5 Subset

| | | | RMSD |
|---|---|---|---|
| | | | from |
| Molecule # | pdb | type | Family Avg. |
| 1 | 1A80 | 2,5-Diketo-D- | 0.21 |
| | | Gluconic Acid | |
| | | Reductase | |
| | | (Cornybacterium) | |
| 2 | 1AFS | 3-a-Hydroxysteroid Dehydro- | 0.66 |
| | | genase (rat) | |
| 3 | 1FRB | Aldo-Keto Reductase (mouse) | 0.55 |
| 4 | 1ADS | Aldose Reductase (human) | 0.55 |
| 5 | 1AH0 | Aldose Reductase (pig) | 0.56 |

TABLE 7B
Polypeptide and Solvent Interactors (average coordinates)

| atom | residue- | | | | | | | |
|---|---|---|---|---|---|---|---|---|
| name | mol. # | total | x | σx | y | σy | z | σz |
| Acceptors | | | | | | | | |
| A3 | ACC | 5 | −0.31 | 0.38 | 8.08 | 0.84 | −3.93 | 0.51 |
| A5 | ACC | 5 | −7.54 | 0.31 | 10.00 | 0.16 | 0.36 | 0.24 |
| A8 (D6) | ACC | 5 | −3.86 | 0.33 | 10.11 | 0.12 | 2.13 | 0.21 |
| A11 (D11) | ACC | 5 | −3.42 | 0.36 | 10.75 | 0.31 | 6.12 | 0.36 |
| A14 (D15) | ACC | 5 | −7.65 | 0.42 | 8.35 | 0.28 | 7.93 | 0.19 |
| A18 | ACC | 5 | −8.07 | 0.25 | 7.90 | 0.12 | 3.55 | 0.09 |
| A32 (D35) | ACC | 5 | −3.37 | 0.49 | 3.38 | 0.29 | −11.88 | 0.27 |
| A37 | ACC | 5 | −6.70 | 0.49 | −3.63 | 0.36 | −15.32 | 0.27 |
| A38 | ACC | 5 | −7.25 | 0.30 | −4.35 | 0.17 | −13.39 | 0.20 |
| A40 | ACC | 4 | −8.26 | 0.22 | −0.78 | 0.09 | −10.85 | 0.30 |
| A42 (D21) | ACC | 4 | −4.11 | 0.29 | 3.97 | 0.06 | 7.45 | 0.05 |
| A43 (D49) | ACC | 4 | −3.07 | 0.46 | 1.67 | 0.40 | 1.87 | 0.38 |
| A55 (D65) | ACC | 3 | 0.11 | 0.37 | 1.66 | 0.18 | −0.35 | 0.22 |
| A58 | ACC | 3 | 1.32 | 0.18 | 2.39 | 0.11 | −4.18 | 0.31 |
| A59 | ACC | 3 | 1.96 | 0.22 | 4.01 | 0.11 | 5.47 | 0.31 |
| Donors | | | | | | | | |
| D2 | DON | 5 | −4.83 | 0.41 | 9.93 | 0.42 | −4.13 | 0.06 |
| D3 | DON | 5 | −2.29 | 0.33 | 9.76 | 0.48 | −2.96 | 0.18 |
| D6 (A8) | DON | 5 | −3.86 | 0.33 | 10.11 | 0.12 | 2.13 | 0.21 |
| D11 (A11) | DON | 5 | −3.42 | 0.36 | 10.75 | 0.31 | 6.12 | 0.36 |
| D15 (A14) | DON | 5 | −7.65 | 0.42 | 8.35 | 0.28 | 7.93 | 0.19 |
| D17 | DON | 5 | −4.88 | 0.29 | 7.13 | 0.34 | 9.26 | 0.08 |
| D21 (A42) | DON | 5 | −4.42 | 0.74 | 4.02 | 0.11 | 7.28 | 0.3 |
| D22 | DON | 5 | −5.81 | 0.30 | 1.79 | 0.28 | 0.94 | 0.10 |
| D24 | DON | 5 | −5.85 | 0.17 | −2.29 | 0.15 | −2.39 | 0.10 |
| D26 | DON | 5 | −1.59 | 0.17 | −1.52 | 0.26 | −1.17 | 0.14 |
| D27 | DON | 1 | −0.90 | — | 2.47 | — | 1.79 | — |
| D32 | DON | 5 | −5.76 | 0.30 | 3.99 | 0.12 | −5.84 | 0.34 |
| D35 (A32) | DON | 5 | −3.37 | 0.49 | 3.38 | 0.29 | −11.88 | 0.27 |
| D36 | DON | 5 | −1.89 | 0.69 | 6.00 | 0.37 | −11.25 | 0.14 |
| D43 | DON | 5 | 0.35 | 0.44 | 0.04 | 0.54 | −12.44 | 0.04 |
| D47 | DON | 4 | −7.47 | 0.24 | 1.06 | 0.13 | −9.91 | 0.26 |
| D49 (A43) | DON | 4 | −3.07 | 0.46 | 1.67 | 0.40 | 1.87 | 0.38 |
| D64 | DON | 3 | 0.37 | 0.27 | 4.92 | 0.07 | −3.02 | 0.15 |
| D65 (A55) | DON | 3 | 0.11 | 0.37 | 1.66 | 0.18 | −0.35 | 0.22 |
| Waters | | | | | | | | |
| W1 | WAT | 4 | 0.62 | 0.21 | −3.17 | 0.55 | −8.81 | 0.66 |
| W9 | WAT | 4 | 2.90 | 0.30 | 3.03 | 0.33 | −8.84 | 0.37 |

TABLE 7C
NAD(P) Conformer Model

| atom name | total | x | σx | y | σy | z | σz |
|---|---|---|---|---|---|---|---|
| PA | 5 | −3.59 | 0.07 | 1.15 | 0.06 | −3.16 | 0.09 |
| O1A | 5 | −3.91 | 0.07 | −0.06 | 0.08 | −2.37 | 0.06 |
| O2A | 5 | −4.70 | 0.10 | 1.87 | 0.11 | −3.82 | 0.09 |
| O5′A | 5 | −2.52 | 0.10 | 0.72 | 0.06 | −4.25 | 0.09 |
| C5′A | 5 | −1.97 | 0.11 | 1.62 | 0.06 | −5.21 | 0.09 |
| C4′A | 5 | −1.00 | 0.13 | 0.82 | 0.07 | −6.06 | 0.07 |
| O4′A | 5 | −1.74 | 0.17 | −0.16 | 0.08 | −6.80 | 0.06 |
| C3′A | 5 | −0.24 | 0.20 | 1.65 | 0.08 | −7.07 | 0.11 |
| O3′A | 5 | 1.09 | 0.17 | 1.16 | 0.21 | −7.14 | 0.19 |
| C2′A | 5 | −0.96 | 0.21 | 1.42 | 0.12 | −8.38 | 0.08 |
| O2′A | 5 | −0.03 | 0.25 | 1.44 | 0.24 | −9.46 | 0.12 |
| C1′A | 5 | −1.49 | 0.16 | 0.01 | 0.09 | −8.20 | 0.07 |
| N9A | 5 | −2.74 | 0.16 | −0.23 | 0.11 | −8.94 | 0.08 |
| C8A | 5 | −3.87 | 0.15 | 0.51 | 0.05 | −9.04 | 0.13 |
| N7A | 5 | −4.77 | 0.16 | −0.07 | 0.05 | −9.80 | 0.19 |
| C5A | 5 | −4.20 | 0.14 | −1.23 | 0.09 | −10.20 | 0.13 |
| C6A | 5 | −4.67 | 0.20 | −2.26 | 0.14 | −11.02 | 0.14 |
| N6A | 5 | −5.88 | 0.24 | −2.27 | 0.19 | −11.55 | 0.20 |
| N1A | 5 | −3.84 | 0.23 | −3.30 | 0.17 | −11.24 | 0.14 |
| C2A | 5 | −2.64 | 0.22 | −3.33 | 0.19 | −10.69 | 0.18 |
| N3A | 5 | −2.13 | 0.23 | −2.39 | 0.17 | −9.90 | 0.15 |
| C4A | 5 | −2.94 | 0.14 | −1.35 | 0.12 | −9.67 | 0.08 |
| O3 | 5 | −2.67 | 0.10 | 2.02 | 0.11 | −2.19 | 0.13 |
| PN | 5 | −2.64 | 0.33 | 3.48 | 0.09 | −1.61 | 0.18 |
| O2N | 5 | −1.78 | 0.43 | 3.39 | 0.25 | −0.42 | 0.27 |
| O1N | 5 | −2.28 | 0.39 | 4.43 | 0.23 | −2.64 | 0.37 |
| O5′N | 5 | −4.08 | 0.45 | 3.75 | 0.33 | −1.10 | 0.12 |
| C5′N | 5 | −5.08 | 0.40 | 4.38 | 0.23 | −1.89 | 0.10 |
| C4′N | 5 | −5.43 | 0.23 | 5.74 | 0.13 | −1.36 | 0.03 |
| O4′N | 5 | −5.93 | 0.16 | 5.65 | 0.12 | −0.02 | 0.04 |
| C3′N | 5 | −4.26 | 0.18 | 6.68 | 0.23 | −1.23 | 0.10 |
| O3′N | 5 | −3.85 | 0.24 | 7.22 | 0.37 | −2.47 | 0.14 |
| C2′N | 5 | −4.83 | 0.19 | 7.72 | 0.11 | −0.32 | 0.12 |
| O2′N | 5 | −5.69 | 0.24 | 8.58 | 0.11 | −1.05 | 0.14 |
| C1′N | 5 | −5.61 | 0.09 | 6.86 | 0.10 | 0.66 | 0.03 |
| N1N | 5 | −4.82 | 0.08 | 6.56 | 0.06 | 1.86 | 0.06 |
| C2N | 5 | −5.21 | 0.09 | 7.16 | 0.08 | 3.04 | 0.07 |
| C3N | 5 | −4.46 | 0.11 | 6.94 | 0.05 | 4.21 | 0.09 |
| C7N | 5 | −4.88 | 0.17 | 7.54 | 0.12 | 5.51 | 0.09 |
| O7N | 5 | −4.17 | 0.19 | 7.45 | 0.25 | 6.50 | 0.12 |
| N7N | 5 | −6.04 | 0.21 | 8.19 | 0.19 | 5.56 | 0.07 |
| C4N | 5 | −3.34 | 0.13 | 6.14 | 0.07 | 4.16 | 0.09 |
| C5N | 5 | −2.95 | 0.14 | 5.55 | 0.14 | 2.98 | 0.11 |
| C6N | 5 | −3.70 | 0.10 | 5.76 | 0.14 | 1.84 | 0.10 |
| P2′ | 5 | −0.06 | 0.34 | 2.60 | 0.41 | −10.53 | 0.12 |
| OP1 | 5 | −0.27 | 0.66 | 3.20 | 0.94 | −10.55 | 0.97 |
| OP2 | 5 | 0.89 | 1.15 | 2.72 | 0.92 | −10.83 | 0.65 |
| OP3 | 5 | −0.55 | 0.81 | 2.71 | 0.77 | −11.09 | 0.69 |

TABLE 7D
Polypeptide and Solvent Interactors

| | residue- | | | | | | | | |
|---|---|---|---|---|---|---|---|---|---|
| atom name | mol. # | residue # | total | x | σx | y | σy | z | σz |
| Acceptors | | | | | | | | | |
| O | PHE 1 | 22 | | −0.22 | | 7.917 | | −3.902 | |
| O | THR 2 | 24 | | −0.117 | | 9.552 | | −4.723 | |
| O | TRP 3 | 20 | | −0.078 | | 7.638 | | −3.451 | |
| O | TRP 4 | 20 | | −0.136 | | 7.449 | | −3.508 | |
| O | TRP 5 | 20 | | −0.979 | | 7.848 | | −4.071 | |
| A3 | ACC | 3 | 5 | −0.306 | 0.37978 | 8.0808 | 0.842719 | −3.931 | 0.51406 |
| OD1 | ASP 1 | 45 | | −7.465 | | 10.181 | | 0.624 | |
| OD2 | ASP 2 | 50 | | −7.821 | | 9.947 | | 0.608 | |
| OD2 | ASP 3 | 43 | | −7.26 | | 10.05 | | 0.226 | |
| OD2 | ASP 4 | 43 | | −7.257 | | 10.064 | | 0.178 | |
| OD2 | ASP 5 | 43 | | −7.906 | | 9.75 | | 0.15 | |
| A5 | ACC | 5 | 5 | −7.542 | 0.30701 | 9.9984 | 0.161751 | 0.3572 | 0.23788 |
| OH | TYR 1 | 50 | | −3.489 | | 9.992 | | 2.109 | |
| OH | TYR 2 | 55 | | −4.193 | | 10.25 | | 2.441 | |
| OH | TYR 3 | 48 | | −3.749 | | 9.978 | | 2.218 | |
| OH | TYR 4 | 48 | | −3.652 | | 10.133 | | 1.976 | |
| OH | TYR 5 | 48 | | −4.239 | | 10.209 | | 1.899 | |
| A8 | ACC | 8 | 5 | −3.864 | 0.33454 | 10.112 | 0.123743 | 2.1286 | 0.21329 |
| NE2 | HIS 1 | 108 | | −3.007 | | 10.311 | | 6.445 | |
| NE2 | HIS 2 | 117 | | −3.912 | | 10.667 | | 6.566 | |
| NE2 | HIS 3 | 110 | | −3.39 | | 11.167 | | 5.845 | |
| NE2 | HIS 4 | 110 | | −3.153 | | 10.889 | | 5.871 | |
| NE2 | HIS 5 | 110 | | −3.636 | | 10.73 | | 5.849 | |
| A11 | ACC | 11 | 5 | −3.42 | 0.36451 | 10.755 | 0.312868 | 6.1152 | 0.35899 |
| OG | SER 1 | 139 | | −7.14 | | 8.138 | | 8.261 | |
| OG | SER 2 | 166 | | −8.27 | | 7.971 | | 7.92 | |
| OG | SER 3 | 159 | | −7.772 | | 8.621 | | 1.778 | |
| OG | SER 4 | 159 | | −7.65 | | 8.495 | | 7.82 | |
| OG | SER 5 | 159 | | −7.437 | | 8.529 | | 7.856 | |
| A14 | ACC | 14 | 5 | −7.654 | 0.41973 | 8.3508 | 0.280664 | 7.927 | 0.19384 |
| OE1 | GLN 1 | 161 | | −7.73 | | 7.828 | | 3.644 | |
| OE1 | GLN 2 | 190 | | −8.407 | | 7.736 | | 3.471 | |
| OE1 | GLN 3 | 183 | | −8.012 | | 8.025 | | 3.461 | |
| OE1 | GLN 4 | 183 | | −8.028 | | 7.965 | | 3.514 | |
| OE1 | GLN 5 | 183 | | −8.175 | | 7.938 | | 3.638 | |
| A18 | ACC | 18 | 5 | −8.07 | 0.24765 | 7.8984 | 0.1155 | 3.5456 | 0.08936 |
| OG | SER 1 | 233 | | −2.688 | | 3.039 | | −11.94 | |
| OG | SER 2 | 271 | | −3.273 | | 3.123 | | −12.31 | |
| OG | SER 3 | 263 | | −3.404 | | 3.664 | | −11.79 | |
| OG | SER 4 | 263 | | −3.447 | | 3.654 | | −11.8 | |
| OG | SER 5 | 263 | | −4.061 | | 3.397 | | −11.59 | |
| A32 | ACC | 32 | 5 | −3.375 | 0.48964 | 3.3754 | 0.290794 | −11.88 | 0.27029 |
| OE1 | GLU 1 | 241 | | −6.654 | | −3.242 | | −15.12 | |
| OE1 | GLU 2 | 279 | | −6.05 | | −4.113 | | −15.74 | |
| OE1 | GLU 3 | 271 | | −6.813 | | −3.347 | | −15.07 | |
| OE1 | GLU 4 | 271 | | −6.579 | | −3.598 | | −15.29 | |
| OE1 | GLU 5 | 271 | | −7.419 | | −3.871 | | −15.4 | |
| A37 | ACC | 37 | 5 | −6.703 | 0.49217 | −3.634 | 0.361573 | −15.32 | 0.26598 |
| OE2 | GLU 1 | 241 | | −7.599 | | −4.219 | | −13.37 | |
| OE2 | GLU 2 | 279 | | −6.79 | | −4.645 | | −13.74 | |
| OE2 | GLU 3 | 271 | | −7.422 | | −4.351 | | −13.25 | |
| OE2 | GLU 4 | 271 | | −7.243 | | −4.266 | | −13.32 | |
| OE2 | GLU 5 | 271 | | −7.176 | | −4.27 | | −13.3 | |
| A38 | ACC | 38 | 5 | −7.246 | 0.30349 | −4.35 | 0.171495 | −13.39 | 0.19848 |
| OD1 | ASN 1 | 242 | | −8.167 | | −0.847 | | −11.28 | |
| OD1 | ASN 3 | 272 | | −8.198 | | −0.802 | | −10.63 | |
| OD1 | ASN 4 | 272 | | −8.082 | | −0.656 | | −10.87 | |
| OD1 | ASN 5 | 272 | | −8.588 | | −0.828 | | −10.63 | |
| A40 | ACC | 40 | 4 | −8.259 | 0.22491 | −0.783 | 0.086815 | −10.85 | 0.30469 |
| OH | TYR 2 | 216 | | −4.48 | | 3.904 | | 7.523 | |
| OH | TYR 3 | 209 | | −4.079 | | 3.966 | | 7.44 | |
| OH | TYR 4 | 209 | | −4.093 | | 4.039 | | 7.418 | |
| OH | TYR 5 | 209 | | −3.784 | | 3.971 | | 7.417 | |
| A42 | ACC | 42 | 4 | −4.109 | 0.28544 | 3.97 | 0.055178 | 7.4495 | 0.05014 |
| SG | CYS 2 | 217 | | −2.381 | | 1.081 | | 2.263 | |
| OG | SER 3 | 210 | | −3.198 | | 1.802 | | 1.827 | |
| OG | SER 4 | 210 | | −3.328 | | 1.843 | | 2.013 | |
| OG | SER 5 | 210 | | −3.366 | | 1.953 | | 1.365 | |
| A43 | ACC | 43 | 4 | −3.068 | 0.46378 | 1.6698 | 0.397644 | 1.867 | 0.37936 |
| OG | SER 3 | 214 | | 0.302 | | 1.569 | | −0.171 | |
| OG | SER 4 | 214 | | 0.348 | | 1.533 | | −0.286 | |
| OG | SER 5 | 214 | | −0.31 | | 1.864 | | −0.589 | |
| A55 | ACC | 55 | 3 | 0.1133 | 0.36734 | 1.6553 | 0.181605 | −0.349 | 0.21593 |
| OD1 | ASP 3 | 216 | | 1.445 | | 2.279 | | −4.029 | |
| OD1 | ASP 4 | 216 | | 1.393 | | 2.409 | | −3.965 | |
| OD1 | ASP 5 | 216 | | 1.107 | | 2.494 | | −4.537 | |
| A58 | ACC | 58 | 3 | 1.315 | 0.182 | 2.394 | 0.108282 | −4.177 | 0.31341 |
| OD2 | ASP 3 | 216 | | 2.06 | | 3.9 | | −5.346 | |
| OD2 | ASP 4 | 216 | | 2.112 | | 3.991 | | −5.233 | |
| OD2 | ASP 5 | 216 | | 1.712 | | 4.127 | | −5.826 | |
| A59 | ACC | 59 | 3 | 1.9613 | 0.21749 | 4.006 | 0.114241 | −5.468 | 0.31486 |
| Donors | | | | | | | | | |
| N | VAL 1 | 21 | | −4.573 | | 10.277 | | −4.214 | |
| N | THR 2 | 23 | | −4.955 | | 10.482 | | −4.051 | |
| N | THR 3 | 19 | | −4.601 | | 9.587 | | −4.125 | |
| N | THR 4 | 19 | | −4.539 | | 9.637 | | −4.107 | |
| N | THR 5 | 19 | | −5.495 | | 9.654 | | −4.137 | |
| D2 | DON | 2 | 5 | −4.833 | 0.40651 | 9.9274 | 0.419748 | −4.127 | 0.05884 |
| N | PHE 1 | 22 | | −2.163 | | 9.689 | | −2.98 | |
| N | THR 2 | 24 | | −2.234 | | 10.595 | | −3.208 | |
| N | TRP 3 | 20 | | −2.126 | | 9.537 | | −2.765 | |
| N | TRP 4 | 20 | | −2.061 | | 9.403 | | −2.815 | |
| N | TRP 5 | 20 | | −2.861 | | 9.571 | | −3.033 | |
| D3 | DON | 3 | 5 | −2.289 | 0.32582 | 9.759 | 0.47832 | −2.96 | 0.17768 |
| OH | TYR 1 | 50 | | −3.489 | | 9.992 | | 2.109 | |
| OH | TYR 2 | 55 | | −4.193 | | 10.25 | | 2.441 | |
| OH | TYR 3 | 48 | | −3.749 | | 9.978 | | 2.218 | |
| OH | TYR 4 | 48 | | −3.652 | | 10.133 | | 1.976 | |
| OH | TYR 5 | 48 | | −4.239 | | 10.209 | | 1.899 | |
| D6 | DON | 6 | 5 | −3.864 | 0.33454 | 10.112 | 0.123743 | 2.1286 | 0.21329 |
| NE2 | HIS 1 | 108 | | −3.007 | | 10.311 | | 6.445 | |
| NE2 | HIS 2 | 117 | | −3.912 | | 10.677 | | 6.566 | |
| NE2 | HIS 3 | 110 | | −3.391 | | 11.167 | | 5.845 | |
| NE2 | HIS 4 | 110 | | −3.153 | | 10.889 | | 5.871 | |
| NE2 | HIS 5 | 110 | | −3.636 | | 10.73 | | 5.849 | |
| D11 | DON | 11 | 5 | −3.42 | 0.36451 | 10.755 | 0.312868 | 6.1152 | 0.35899 |
| OG | SER 1 | 139 | | −7.14 | | 8.138 | | 8.261 | |
| OG | SER 2 | 166 | | −8.27 | | 7.971 | | 7.92 | |
| OG | SER 3 | 159 | | −7.772 | | 8.621 | | 7.778 | |
| OG | SER 4 | 159 | | −7.65 | | 8.495 | | 7.82 | |
| OG | SER 5 | 159 | | −7.437 | | 8.529 | | 7.856 | |
| D15 | DON | 15 | 5 | −7.654 | 0.41973 | 8.3508 | 0.280664 | 7.927 | 0.19384 |
| ND2 | ASN 1 | 140 | | −4.533 | | 6.58 | | 9.266 | |
| ND2 | ASN 2 | 167 | | −5.286 | | 7.047 | | 9.369 | |
| ND2 | ASN 3 | 160 | | −4.994 | | 7.442 | | 9.225 | |
| ND2 | ASN 4 | 160 | | −4.894 | | 7.259 | | 9.278 | |
| ND2 | ASN 5 | 160 | | −4.669 | | 7.311 | | 9.151 | |
| D17 | DON | 17 | 5 | −4.875 | 0.29276 | 7.1278 | 0.33768 | 9.2578 | 0.07957 |
| NE1 | TRP 1 | 187 | | −5.659 | | 4.197 | | 6.593 | |
| OH | TYR 2 | 216 | | −4.48 | | 3.904 | | 7.523 | |
| OH | TYR 3 | 209 | | −4.079 | | 3.966 | | 7.44 | |
| OH | TYR 4 | 209 | | −4.093 | | 4.039 | | 7.418 | |
| OH | TYR 5 | 209 | | −3.784 | | 3.971 | | 7.417 | |
| D21 | DON | 21 | 5 | −4.419 | 0.73594 | 4.0154 | 0.112202 | 7.2782 | 0.38549 |
| N | GLY 1 | 188 | | −5.543 | | 1.806 | | 1.07 | |
| N | CYS 2 | 217 | | −5.457 | | 1.307 | | 0.834 | |
| N | SER 3 | 210 | | −5.913 | | 2.008 | | 0.883 | |
| N | SER 4 | 210 | | −5.995 | | 1.926 | | 1.01 | |
| N | SER 5 | 210 | | −6.138 | | 1.889 | | 0.879 | |
| D22 | DON | 22 | 5 | −5.809 | 0.29509 | 1.7872 | 0.278086 | 0.9352 | 0.09986 |
| N | LEU 1 | 190 | | −6.122 | | −2.167 | | −2.319 | |
| N | LEU 2 | 219 | | −5.697 | | −2.431 | | −2.521 | |
| N | LEU 3 | 212 | | −5.848 | | −2.116 | | −2.486 | |
| N | LEU 4 | 212 | | −5.837 | | −2.313 | | −2.318 | |
| N | LEU 5 | 212 | | −5.738 | | −2.444 | | −2.315 | |
| D24 | DON | 24 | 5 | −5.848 | 0.1659 | −2.294 | 0.149535 | −2.392 | 0.10273 |
| N | GLN 1 | 192 | | −1.835 | | −1.942 | | −1.288 | |
| N | SER 2 | 221 | | −1.633 | | −1.501 | | −0.943 | |
| N | SER 3 | 214 | | −1.557 | | −1.387 | | −1.269 | |
| N | SER 4 | 214 | | −1.543 | | −1.524 | | −1.135 | |
| N | SER 5 | 214 | | −1.368 | | −1.233 | | −1.228 | |
| D26 | DON | 26 | 5 | −1.587 | 0.16913 | −1.517 | 0.263858 | −1.173 | 0.14125 |
| NE2 | GLN 1 | 192 | | −0.903 | | 2.473 | | 1.785 | |
| D27 | DON | 27 | 1 | −0.903 | | 2.473 | | 1.785 | |
| N | LYS 1 | 232 | | −5.402 | | 4.166 | | −6.054 | |
| N | ARG 2 | 270 | | −5.952 | | 3.855 | | −6.343 | |
| N | LYS 3 | 262 | | −5.685 | | 4.007 | | −5.639 | |
| N | LYS 4 | 262 | | −5.623 | | 3.992 | | −5.582 | |
| N | LYS 5 | 262 | | −6.162 | | 3.913 | | −5.584 | |
| D32 | DON | 32 | 5 | −5.765 | 0.29619 | 3.9866 | 0.117649 | −5.84 | 0.34326 |
| OG | SER 1 | 233 | | −2.688 | | 3.039 | | −11.94 | |
| OG | SER 2 | 271 | | −3.273 | | 3.123 | | −12.31 | |
| OG | SER 3 | 263 | | −3.404 | | 3.664 | | −11.79 | |
| OG | SER 4 | 263 | | −3.447 | | 3.654 | | −11.8 | |
| OG | SER 5 | 263 | | −4.061 | | 3.397 | | −11.59 | |
| D35 | DON | 35 | 5 | −3.375 | 0.48964 | 3.3754 | 0.290794 | −11.88 | 0.27029 |
| N | VAL 1 | 234 | | −1.14 | | 5.556 | | −11.43 | |
| N | PHE 2 | 272 | | −1.614 | | 5.656 | | −11.37 | |
| N | VAL 3 | 264 | | −1.81 | | 6.206 | | −11.19 | |
| N | VAL 4 | 264 | | −1.882 | | 6.219 | | −11.12 | |
| N | VAL 5 | 264 | | −3.012 | | 6.373 | | −11.15 | |
| D36 | DON | 36 | 5 | −1.892 | 0.68993 | 6.002 | 0.369113 | −11.25 | 0.13745 |
| NH1 | ARG 1 | 238 | | 0.069 | | −0.686 | | −12 | |
| NH2 | ARG 2 | 276 | | 1.098 | | 0.722 | | −13.92 | |
| NH1 | ARG 3 | 268 | | 0.415 | | 0.209 | | −12.73 | |
| NH1 | ARG 4 | 268 | | 0.039 | | −0.27 | | −11.5 | |
| NH2 | ARG 5 | 268 | | 0.142 | | 0.24 | | −12.05 | |
| D43 | DON | 43 | 4 | 0.3526 | 0.44234 | 0.043 | 0.537777 | −12.44 | 0.93623 |
| ND2 | ASN 1 | 242 | | −7.301 | | 0.978 | | −10.22 | |
| ND2 | ASN 3 | 272 | | −7.385 | | 1.094 | | −9.791 | |
| ND2 | ASN 4 | 272 | | −7.367 | | 1.218 | | −10.01 | |
| ND2 | ASN 5 | 272 | | −7.832 | | 0.939 | | −9.618 | |
| D47 | DON | 47 | 4 | −7.471 | 0.2432 | 1.0573 | 0.125771 | −9.91 | 0.26174 |
| SG | CYS 2 | 217 | | −2.381 | | 1.081 | | 2.263 | |
| OG | SER 3 | 210 | | −3.198 | | 1.802 | | 1.827 | |
| OG | SER 4 | 210 | | −3.328 | | 1.843 | | 2.013 | |
| OG | SER 5 | 210 | | −3.366 | | 1.953 | | 1.365 | |
| D49 | DON | 49 | 4 | −3.068 | 0.46378 | 1.6698 | 0.397644 | 1.867 | 0.37936 |
| NZ | LYS 3 | 21 | | 0.563 | | 4.894 | | −2.898 | |
| NZ | LYS 4 | 21 | | 0.487 | | 4.857 | | −2.975 | |
| NZ | LYS 5 | 21 | | 0.06 | | 4.999 | | −3.187 | |
| D64 | DON | 64 | 3 | 0.37 | 0.27114 | 4.9167 | 0.073664 | −3.02 | 0.14966 |
| OG | SER 3 | 214 | | 0.302 | | 1.569 | | −0.171 | |
| OG | SER 4 | 214 | | 0.348 | | 1.533 | | −0.286 | |
| OG | SER 5 | 214 | | 0.31 | | 1.864 | | −0.589 | |
| D65 | DON | 65 | 3 | 0.1133 | 0.36734 | 1.6553 | 0.181605 | −0.349 | 0.21593 |
| Waters | | | | | | | | | |
| O | HOH 1 | 396 | | 3.263 | | 2.796 | | −9.047 | |
| O | HOH 3 | 536 | | 3.02 | | 2.698 | | −8.645 | |
| O | HOH 4 | 484 | | 2.686 | | 3.261 | | −8.435 | |
| O | HOH 5 | 586 | | 2.613 | | 3.35 | | −9.237 | |
| W9 | WAT | 9 | 4 | 2.895 | 0.30235 | 3.026 | 0.326948 | −8.841 | 0.36629 |
| O | HOH 1 | 307 | | 0.306 | | −3.84 | | −7.869 | |
| O | HOH 3 | 731 | | 0.694 | | −3.294 | | −8.887 | |
| O | HOH 4 | 485 | | 0.782 | | −3.008 | | −9.378 | |
| O | HOH 5 | 483 | | 0.686 | | −2.519 | | −9.123 | |
| W1 | WAT | 1 | 4 | 0.617 | 0.21185 | −3.165 | 0.552036 | −8.814 | 0.66129 |

TABLE 8A
Pharmacofamily 6 Subset

| | | | RMSD from |
|---|---|---|---|
| Molecule # | pdb | type | Family Avg. |
| 1 | 1AI9 | Dihydrofolate Reductase | 0.49 |
| | | (candida albicans) | |
| 2 | 1DAJ | DHFR (pneumocystis carinii) | 0.8 |
| 3 | 1DLR | DHFR (human) | |
| 4 | 1DR1 | DHFR (chicken) | 0.83 |
| 5 | 1DRE | DHFR (E. coli) | 0.91 |
| 6 | 3DFR | DHFR (Lactobacillus casei) | 0.84 |

TABLE 8B
Polypeptide and Solvent Interactors (average coordinates)

| atom name | Name | total | x | σx | y | σy | z | σz |
|---|---|---|---|---|---|---|---|---|
| Acceptors | | | | | | | | |
| A2 | ACC | 6 | −7.76 | 0.34 | 9.50 | 0.60 | 15.24 | 0.31 |
| A3 | ACC | 6 | −3.33 | 0.36 | 9.00 | 0.28 | 13.41 | 0.29 |
| A7 | ACC | 6 | 4.38 | 0.42 | 8.51 | 0.59 | 14.79 | 0.44 |
| A8 | ACC | 5 | 0.64 | 0.44 | 10.67 | 0.55 | 12.99 | 0.29 |
| A22 | ACC | 5 | 1.78 | 0.52 | −12.11 | 0.61 | 17.27 | 0.35 |
| A29 | ACC | 3 | 1.38 | 0.22 | −3.65 | 0.98 | 10.30 | 0.42 |
| A45 (D53) | ACC | 5 | 7.52 | 0.32 | −6.82 | 0.15 | 17.60 | 0.52 |
| A64 | ACC | 1 | 3.88 | | 7.64 | | 10.73 | |
| Donors | | | | | | | | |
| D2 | DON | 6 | −8.77 | 0.24 | 8.47 | 0.48 | 17.58 | 0.39 |
| D5 | DON | 6 | 0.31 | 0.46 | 10.32 | 0.28 | 10.41 | 0.31 |
| D7 | DON | 6 | 4.49 | 0.64 | 8.48 | 0.37 | 11.28 | 0.47 |
| D8 | DON | 6 | 3.29 | 0.49 | 9.75 | 0.37 | 13.31 | 0.28 |
| D10 | DON | 6 | 0.75 | 0.68 | 11.75 | 0.20 | 14.90 | 0.31 |
| D13 | DON | 6 | 0.42 | 0.31 | −1.68 | 0.29 | 18.99 | 0.21 |
| D14 | DON | 6 | 3.77 | 0.31 | −2.26 | 0.30 | 17.84 | 0.28 |
| D15 | DON | 6 | 9.09 | 0.30 | −3.80 | 0.34 | 14.68 | 0.76 |
| D18 | DON | 6 | 4.89 | 0.37 | 0.01 | 0.38 | 16.50 | 0.32 |
| D19 | DON | 6 | 5.76 | 0.34 | −0.45 | 1.23 | 11.73 | 0.51 |
| D20 | DON | 6 | 3.21 | 0.48 | 2.15 | 0.27 | 17.41 | 0.31 |
| D24 | DON | 6 | 8.21 | 0.50 | −9.32 | 0.64 | 16.12 | 0.77 |
| D25 | DON | 6 | 5.73 | 0.39 | −9.28 | 0.30 | 16.15 | 0.47 |
| D27 | DON | 6 | 4.63 | 0.21 | −8.88 | 0.26 | 11.81 | 0.22 |
| D35 | DON | 6 | −1.87 | 0.34 | 0.75 | 0.49 | 16.42 | 0.33 |
| D3 7 | DON | 6 | −2.91 | 0.56 | −1.48 | 0.83 | 11.81 | 0.33 |
| D38 | DON | 6 | −3.30 | 0.47 | −3.07 | 0.64 | 14.06 | 0.39 |
| D40 | DON | 6 | −6.32 | 0.26 | 3.86 | 0.48 | 17.78 | 0.67 |
| D53(A45) | DON | 6 | 7.52 | 0.32 | −6.82 | 0.15 | 17.60 | 0.52 |
| D58 | DON | 6 | 4.59 | 0.01 | 4.70 | 0.53 | 10.76 | 0.38 |
| Waters | | | | | | | | |
| W5 | WAT | 3 | 3.12 | 0.69 | 4.35 | 0.33 | 10.23 | 0.39 |
| W7 | WAT | 3 | 2.33 | 0.11 | 6.97 | 0.14 | 10.21 | 0.07 |
| W9 | WAT | 2 | 1.38 | 0.94 | 3.27 | 0.01 | 9.07 | 0.57 |
| W10 | WAT | 3 | −2.58 | 0.27 | −11.63 | 0.89 | 15.29 | 0.33 |

TABLE 8C
NAD(P) Conformer Model

| atom name | total | x | σx | y | σy | z | σz |
|---|---|---|---|---|---|---|---|
| PA | 6 | 1.05 | 0.24 | −0.17 | 0.19 | 14.67 | 0.19 |
| O1A | 6 | 1.19 | 0.24 | 0.64 | 0.25 | 15.88 | 0.23 |
| O2A | 6 | −0.20 | 0.24 | −0.90 | 0.28 | 14.47 | 0.18 |
| O5′A | 6 | 2.35 | 0.21 | −1.13 | 0.14 | 14.56 | 0.24 |
| C5′A | 6 | 2.40 | 0.23 | −2.23 | 0.10 | 13.62 | 0.23 |
| C4′A | 6 | 3.42 | 0.23 | −3.27 | 0.14 | 14.17 | 0.18 |
| O4′A | 6 | 2.79 | 0.36 | −3.93 | 0.29 | 15.07 | 0.24 |
| C3′A | 6 | 3.64 | 0.12 | −4.36 | 0.13 | 13.07 | 0.19 |
| O3′A | 6 | 4.70 | 0.13 | −3.76 | 0.25 | 12.26 | 0.24 |
| C2′A | 6 | 4.06 | 0.05 | −5.51 | 0.17 | 14.00 | 0.26 |
| O2′A | 6 | 5.31 | 0.06 | −5.32 | 0.34 | 14.57 | 0.28 |
| C1′A | 6 | 3.05 | 0.11 | −5.32 | 0.22 | 15.11 | 0.22 |
| N9A | 6 | 1.81 | 0.09 | −5.96 | 0.35 | 14.84 | 0.21 |
| C8A | 6 | 0.76 | 0.17 | −5.40 | 0.56 | 14.27 | 0.47 |
| N7A | 6 | −0.27 | 0.17 | −6.16 | 0.65 | 14.17 | 0.44 |
| C5A | 6 | 0.21 | 0.15 | −7.35 | 0.53 | 14.68 | 0.21 |
| C6A | 6 | −0.44 | 0.24 | −8.68 | 0.51 | 14.89 | 0.32 |
| N6A | 6 | −1.69 | 0.28 | −8.92 | 0.67 | 14.53 | 0.44 |
| N1A | 6 | 0.29 | 0.35 | −9.56 | 0.36 | 15.44 | 0.49 |
| C2A | 6 | 1.54 | 0.34 | −9.19 | 0.25 | 15.79 | 0.52 |
| N3A | 6 | 2.22 | 0.25 | −8.09 | 0.22 | 15.65 | 0.34 |
| C4A | 6 | 1.45 | 0.13 | −7.18 | 0.35 | 15.09 | 0.07 |
| O3 | 6 | 1.42 | 0.24 | 0.75 | 0.10 | 13.47 | 0.20 |
| PN | 6 | 0.72 | 0.34 | 1.45 | 0.19 | 12.25 | 0.14 |
| O1N | 6 | 1.73 | 0.45 | 1.89 | 0.29 | 11.31 | 0.22 |
| O2N | 6 | −0.36 | 0.53 | 0.71 | 0.34 | 11.74 | 0.15 |
| O5′N | 6 | 0.22 | 0.15 | 2.75 | 0.17 | 12.92 | 0.26 |
| C5′N | 6 | 1.01 | 0.12 | 3.77 | 0.28 | 13.48 | 0.39 |
| C4′N | 6 | 0.38 | 0.25 | 5.08 | 0.27 | 13.02 | 0.22 |
| O4′N | 6 | −0.91 | 0.16 | 5.18 | 0.29 | 13.67 | 0.13 |
| C3′N | 6 | 1.12 | 0.29 | 6.33 | 0.23 | 13.52 | 0.32 |
| O3′N | 6 | 1.00 | 0.36 | 7.39 | 0.27 | 12.63 | 0.36 |
| C2′N | 6 | 0.45 | 0.21 | 6.61 | 0.24 | 14.87 | 0.28 |
| O2′N | 6 | 0.66 | 0.31 | 7.95 | 0.27 | 15.21 | 0.40 |
| C1′N | 6 | −0.96 | 0.21 | 6.30 | 0.20 | 14.54 | 0.23 |
| N1N | 6 | −1.94 | 0.08 | 6.13 | 0.21 | 15.69 | 0.16 |
| C2N | 6 | −3.04 | 0.10 | 6.97 | 0.25 | 15.83 | 0.15 |
| C3N | 6 | −3.94 | 0.11 | 6.79 | 0.28 | 16.76 | 0.16 |
| C7N | 6 | −5.03 | 0.17 | 7.76 | 0.42 | 16.79 | 0.23 |
| O7N | 6 | −5.87 | 0.22 | 7.55 | 0.50 | 17.62 | 0.42 |
| N7N | 6 | −5.15 | 0.38 | 8.68 | 0.43 | 15.88 | 0.20 |
| C4N | 6 | −3.80 | 0.33 | 5.71 | 0.33 | 17.78 | 0.25 |
| C5N | 6 | −2.57 | 0.33 | 4.91 | 0.28 | 17.56 | 0.23 |
| C6N | 6 | −1.72 | 0.21 | 5.11 | 0.17 | 16.58 | 0.19 |
| P2′ | 6 | 6.67 | 0.14 | −6.07 | 0.47 | 14.05 | 0.35 |
| OP1 | 6 | 6.95 | 0.63 | −6.04 | 0.74 | 14.07 | 1.55 |
| OP2 | 6 | 6.45 | 0.52 | −7.18 | 0.71 | 13.88 | 0.88 |
| OP3 | 6 | 7.41 | 0.41 | −5.33 | 0.70 | 13.79 | 0.83 |

TABLE 8D
Polypeptide and Solvent Interactors

| | residue- | | | | | | | | |
|---|---|---|---|---|---|---|---|---|---|
| atom name | mol. # | residue # | total | x | σx | y | σy | z | σz |
| Acceptors | | | | | | | | | |
| O | ALA 1 | 11 | | −8.25 | | 9.15 | | 15.70 | |
| O | ALA 2 | 12 | | −7.62 | | 9.56 | | 15.25 | |
| O | ALA 3 | 9 | | −7.84 | | 8.91 | | 15.02 | |
| O | ALA 4 | 9 | | −8.02 | | 9.04 | | 15.08 | |
| O | ALA 5 | 7 | | −7.34 | | 10.51 | | 14.88 | |
| O | ALA 6 | 6 | | −7.50 | | 9.83 | | 15.51 | |
| A2 | ACC | 2 | 6 | −7.76 | 0.34 | 9.50 | 0.60 | 15.24 | 0.31 |
| O | ILE 1 | 19 | | −3.73 | | 9.16 | | 13.34 | |
| O | ILE 2 | 19 | | −3.77 | | 8.82 | | 13.73 | |
| O | ILE 3 | 16 | | −3.18 | | 8.72 | | 13.35 | |
| O | ILE 4 | 16 | | −3.34 | | 8.72 | | 13.44 | |
| O | ILE 5 | 14 | | −2.92 | | 9.18 | | 12.93 | |
| O | ILE 6 | 13 | | −3.03 | | 9.39 | | 13.70 | |
| A3 | ACC | 3 | 6 | −3.33 | 0.36 | 9.00 | 0.28 | 13.41 | 0.29 |
| O | GLY 1 | 23 | | 3.59 | | 8.74 | | 14.29 | |
| O | ASN 2 | 23 | | 4.73 | | 8.14 | | 14.25 | |
| O | GLY 3 | 20 | | 4.28 | | 9.37 | | 15.16 | |
| O | GLY 4 | 20 | | 4.43 | | 8.68 | | 14.84 | |
| O | ASN 5 | 18 | | 4.63 | | 8.52 | | 15.30 | |
| O | GLY 6 | 17 | | 4.64 | | 7.62 | | 14.92 | |
| A7 | ACC | 7 | 6 | 4.38 | 0.42 | 8.51 | 0.59 | 14.79 | 0.44 |
| O | LYS 1 | 24 | | 0.01 | | 11.45 | | 12.52 | |
| O | SER 2 | 24 | | 0.93 | | 11.05 | | 13.09 | |
| O | ASP 3 | 21 | | 0.38 | | 10.26 | | 13.30 | |
| O | ASN 4 | 21 | | 0.78 | | 10.18 | | 13.08 | |
| O | ALA 5 | 19 | | 1.10 | | 10.42 | | 12.96 | |
| A8 | ACC | 8 | 5 | 0.64 | 0.44 | 10.67 | 0.55 | 12.99 | 0.29 |
| OE1 | GLU 1 | 116 | | 1.44 | | −3.73 | | 10.26 | |
| OE1 | GLN 2 | 127 | | 1.14 | | −4.59 | | 10.74 | |
| OE1 | GLN 6 | 101 | | 1.56 | | −2.63 | | 9.89 | |
| A29 | ACC | 29 | 3 | 1.38 | 0.22 | −3.65 | 0.98 | 10.30 | 0.42 |
| OG1 | THR 2 | 81 | | 7.15 | | −6.59 | | 18.23 | |
| OG | SER 3 | 76 | | 7.84 | | −6.95 | | 17.31 | |
| OG | SER 4 | 76 | | 7.83 | | −6.93 | | 16.92 | |
| OG | SER 5 | 63 | | 7.26 | | −6.86 | | 17.98 | |
| OG1 | THR 6 | 63 | | 7.53 | | −6.78 | | 17.57 | |
| A45 | ACC | 45 | 5 | 7.52 | 0.32 | −6.82 | 0.15 | 17.60 | 0.52 |
| O | GLU 5 | 17 | | 3.88 | | 7.64 | | 10.73 | |
| A64 | ACC | 64 | 1 | 3.88 | | 7.64 | | 10.73 | |
| O | SER 1 | 94 | | 1.16 | | −12.13 | | 17.75 | |
| O | LYS 2 | 96 | | 1.98 | | −11.25 | | 17.47 | |
| O | ARG 3 | 91 | | 2.27 | | −12.14 | | 16.86 | |
| O | LYS 4 | 91 | | 2.20 | | −12.05 | | 17.08 | |
| O | LYS 5 | 76 | | 1.29 | | −12.97 | | 17.19 | |
| A22 | ACC | 22 | 5 | 1.78 | 0.52 | −12.11 | 0.61 | 17.27 | 0.35 |
| Donors | | | | | | | | | |
| N | ALA 1 | 11 | | −9.06 | | 8.04 | | 18.17 | |
| N | ALA 2 | 12 | | −8.79 | | 8.01 | | 17.55 | |
| N | ALA 3 | 9 | | −8.95 | | | | 17.22 | |
| N | ALA 4 | 9 | | −8.84 | | 8.16 | | 17.46 | |
| N | ALA 5 | 7 | | −8.61 | | 9.19 | | 17.17 | |
| N | ALA 6 | 6 | | −8.39 | | 8.86 | | 17.88 | |
| D2 | DON | 2 | 6 | −8.77 | 0.24 | 8.45 | 0.54 | 17.58 | 0.39 |
| N | TYR 1 | 21 | | −0.42 | | 10.64 | | 9.86 | |
| N | ARG 2 | 21 | | 0.01 | | 10.40 | | 10.61 | |
| N | LYS 3 | 18 | | 0.40 | | 10.07 | | 10.57 | |
| N | LYS 4 | 18 | | 0.32 | | 9.96 | | 10.47 | |
| N | MET 5 | 16 | | 0.86 | | 10.62 | | 10.25 | |
| N | LYS 6 | 15 | | 0.70 | | 10.26 | | 10.69 | |
| D5 | DON | 5 | 6 | 0.31 | 0.46 | 10.32 | 0.28 | 10.41 | 0.31 |
| N | GLY 1 | 23 | | 3.65 | | 9.06 | | 10.80 | |
| N | ASN 2 | 23 | | 4.05 | | 8.21 | | 10.77 | |
| N | GLY 3 | 20 | | 4.51 | | 8.63 | | 11.63 | |
| N | GLY 4 | 20 | | 4.53 | | 8.63 | | 11.24 | |
| N | ASN 5 | 18 | | 5.57 | | 8.31 | | 11.98 | |
| N | GLY 6 | 17 | | 4.61 | | 8.02 | | 11.26 | |
| D7 | DON | 7 | 6 | 4.49 | 0.64 | 8.48 | 0.37 | 11.28 | 0.47 |
| N | LYS 1 | 24 | | 2.49 | | 10.14 | | 12.86 | |
| N | SER 2 | 24 | | 3.18 | | 9.36 | | 13.12 | |
| N | ASP 3 | 21 | | 3.13 | | 10.15 | | 13.47 | |
| N | ASN 4 | 21 | | 3.34 | | 9.95 | | 13.37 | |
| N | ALA 5 | 19 | | 3.82 | | 9.57 | | 13.45 | |
| N | HIS 6 | 18 | | 3.78 | | 9.34 | | 13.62 | |
| D8 | DON | 8 | 6 | 3.29 | 0.49 | 9.75 | 0.37 | 13.31 | 0.28 |
| N | MET 1 | 25 | | −0.11 | | 11.91 | | 14.72 | |
| N | LEU 2 | 25 | | 1.21 | | 11.60 | | 15.27 | |
| N | PHE 3 | 22 | | 0.10 | | 11.65 | | 14.89 | |
| N | LEU 4 | 22 | | 0.47 | | 11.75 | | 14.68 | |
| N | MET 5 | 20 | | 1.42 | | 12.04 | | 14.55 | |
| N | LEU 6 | 19 | | 1.41 | | 11.53 | | 15.29 | |
| D10 | DON | 10 | 6 | 0.75 | 0.68 | 11.75 | 0.20 | 14.90 | 0.31 |
| N | GLY 1 | 55 | | 0.99 | | −2.06 | | 19.18 | |
| N | GLY 2 | 58 | | 0.23 | | −1.46 | | 19.18 | |
| N | GLY 3 | 53 | | 0.43 | | −1.88 | | 18.67 | |
| N | GLY 4 | 53 | | 0.52 | | −1.82 | | 18.78 | |
| N | GLY 5 | 43 | | 0.23 | | −1.34 | | 19.06 | |
| N | GLY 6 | 42 | | 0.14 | | −1.50 | | 19.06 | |
| D13 | DON | 13 | 6 | 0.42 | 0.31 | −1.68 | 0.29 | 18.99 | 0.21 |
| N | ARG 1 | 56 | | 4.28 | | −2.84 | | 18.05 | |
| N | ARG 2 | 59 | | 3.60 | | −2.00 | | 18.08 | |
| N | LYS 3 | 54 | | 3.84 | | −2.10 | | 17.59 | |
| N | LYS 4 | 54 | | 3.92 | | −2.11 | | 17.43 | |
| N | ARG 5 | 44 | | 3.45 | | −2.27 | | 17.84 | |
| N | ARG 6 | 43 | | 3.51 | | −2.24 | | 18.07 | |
| D14 | DON | 14 | 6 | 3.77 | 0.31 | −2.26 | 0.30 | 17.84 | 0.28 |
| NE | ARG 1 | 56 | | 8.78 | | −3.97 | | 15.50 | |
| NZ | LYS 3 | 54 | | 9.39 | | −3.41 | | 14.54 | |
| NZ | LYS 4 | 54 | | 9.10 | | −4.01 | | 14.01 | |
| D15 | DON | 15 | 3 | 9.09 | 0.30 | −3.80 | 0.34 | 14.68 | 0.76 |
| N | LYS 1 | 57 | | 5.58 | | −0.66 | | 16.65 | |
| N | LYS 2 | 60 | | 4.68 | | 0.38 | | 16.94 | |
| N | LYS 3 | 55 | | 4.80 | | 0.20 | | 16.22 | |
| N | LYS 4 | 55 | | 4.95 | | 0.24 | | 16.06 | |
| N | HIS 5 | 45 | | 4.53 | | 0.07 | | 16.53 | |
| N | ARG 6 | 44 | | 4.80 | | −0.19 | | 16.60 | |
| D18 | DON | 18 | 6 | 4.89 | 0.37 | 0.01 | 0.38 | 16.50 | 0.32 |
| NZ | LYS 1 | 57 | | 6.03 | | −1.79 | | 11.41 | |
| NE2 | HIS 5 | 45 | | 5.83 | | −0.20 | | 12.35 | |
| NE | ARG 6 | 44 | | 5.42 | | 0.63 | | 11.42 | |
| D19 | DON | 19 | 3 | 5.76 | 0.31 | −0.45 | 1.23 | 11.73 | 0.54 |
| N | THR 1 | 58 | | 4.11 | | 1.68 | | 17.55 | |
| N | THR 2 | 61 | | 3.07 | | 2.49 | | 17.92 | |
| N | THR 3 | 56 | | 2.93 | | 2.04 | | 17.18 | |
| N | THR 4 | 56 | | 3.15 | | 2.15 | | 17.06 | |
| N | THR 5 | 46 | | 2.73 | | 2.26 | | 17.40 | |
| N | THR 6 | 45 | | 3.30 | | 2.25 | | 17.33 | |
| D20 | DON | 20 | 6 | 3.21 | 0.48 | 2.15 | 0.27 | 17.41 | 0.31 |
| OG | SER 1 | 78 | | 7.51 | | −8.07 | | 16.81 | |
| N | ASN 2 | 83 | | 7.95 | | −9.42 | | 16.07 | |
| N | GLU 3 | 78 | | 8.83 | | −9.52 | | 15.37 | |
| N | GLU 4 | 78 | | 8.58 | | −9.52 | | 15.10 | |
| N | GLN 5 | 65 | | 7.90 | | −9.91 | | 16.99 | |
| N | GLN 6 | 65 | | 8.50 | | −9.50 | | 16.42 | |
| D24 | DON | 24 | 6 | 8.21 | 0.50 | −9.32 | 0.64 | 16.12 | 0.77 |
| N | ARG 1 | 79 | | 5.13 | | −9.73 | | 15.64 | |
| N | ARG 2 | 82 | | 5.51 | | −9.28 | | 16.87 | |
| N | ARG 3 | 77 | | 6.17 | | −9.41 | | 16.02 | |
| N | ARG 4 | 77 | | 6.01 | | −9.37 | | 15.82 | |
| N | SER 5 | 64 | | 5.59 | | −9.07 | | 16.55 | |
| N | HIS 6 | 64 | | 6.00 | | −8.86 | | 15.99 | |
| D25 | DON | 25 | 6 | 5.73 | 0.39 | −9.28 | 0.30 | 16.15 | 0.47 |
| NH1 | ARG 1 | 79 | | 4.49 | | −8.70 | | 11.66 | |
| NH1 | ARG 2 | 82 | | 4.78 | | −9.07 | | 11.97 | |
| D27 | DON | 27 | 2 | 4.63 | 0.21 | −8.88 | 0.26 | 11.81 | 0.22 |
| N | GLY 1 | 114 | | −1.20 | | 0.66 | | 16.96 | |
| N | GLY 2 | 125 | | −2.08 | | 0.99 | | 16.66 | |
| N | GLY 3 | 117 | | −2.08 | | 0.12 | | 16.11 | |
| N | GLY 4 | 117 | | −2.00 | | 0.26 | | 16.14 | |
| N | GLY 5 | 96 | | −1.87 | | 1.30 | | 16.33 | |
| N | GLY 6 | 99 | | −1.99 | | 1.20 | | 16.31 | |
| D35 | DON | 35 | 6 | −1.87 | 0.34 | 0.75 | 0.49 | 16.42 | 0.33 |
| N | GLU 1 | 116 | | −2.20 | | −0.54 | | 11.97 | |
| N | GLN 2 | 127 | | −2.51 | | −1.22 | | 12.03 | |
| N | SER 3 | 119 | | −3.51 | | −2.29 | | 11.74 | |
| N | ALA 4 | 119 | | −3.63 | | −2.67 | | 11.96 | |
| N | ARG 5 | 98 | | −2.81 | | −0.91 | | 11.18 | |
| N | GLN 6 | 101 | | −2.81 | | −1.25 | | 12.00 | |
| D37 | DON | 37 | 6 | −2.91 | 0.56 | −1.48 | 0.83 | 11.81 | 0.33 |
| N | ILE 1 | 117 | | −2.58 | | −2.52 | | 13.89 | |
| N | LEU 2 | 128 | | −3.06 | | −2.83 | | 14.28 | |
| N | VAL 3 | 120 | | −3.71 | | −3.84 | | 14.05 | |
| N | VAL 4 | 120 | | −3.83 | | −3.92 | | 14.47 | |
| N | VAL 5 | 99 | | −3.54 | | −2.56 | | 13.37 | |
| N | ILE 6 | 102 | | −3.10 | | −2.76 | | 14.27 | |
| D38 | DON | 38 | 6 | −3.30 | 0.47 | −3.07 | 0.64 | 14.06 | 0.39 |
| OH | TYR 1 | 118 | | −5.90 | | 3.87 | | 18.74 | |
| OH | TYR 2 | 129 | | −6.34 | | 4.00 | | 17.96 | |
| OH | TYR 3 | 121 | | −6.27 | | 3.45 | | 17.00 | |
| OH | TYR 4 | 121 | | −6.58 | | 3.42 | | 17.85 | |
| OH | TYR 5 | 100 | | −6.50 | | 4.59 | | 17.32 | |
| D40 | DON | 40 | 5 | −6.32 | 0.26 | 3.86 | 0.48 | 17.78 | 0.67 |
| OG1 | THR 2 | 81 | | 7.15 | | −6.59 | | 18.23 | |
| OG | SER 3 | 76 | | 7.84 | | −6.95 | | 17.31 | |
| OG | SER 4 | 76 | | 7.83 | | −6.93 | | 16.92 | |
| OG | SER 5 | 63 | | 7.26 | | −6.86 | | 17.98 | |
| OG1 | THR 6 | 63 | | 7.53 | | −6.78 | | 17.57 | |
| D53 | DON | 53 | 5 | 7.52 | 0.32 | −6.82 | 0.15 | 17.60 | 0.52 |
| NZ | LYS 3 | 55 | | 4.59 | | 5.07 | | 10.49 | |
| NZ | LYS 4 | 55 | | 4.60 | | 4.32 | | 11.03 | |
| D58 | DON | 58 | 2 | 4.59 | 0.01 | 4.70 | 0.53 | 10.76 | 0.38 |
| Waters | | | | | | | | | |
| O | HOH 1 | 360 | | 3.79 | | 4.24 | | 10.23 | |
| O | HOH 4 | 814 | | 2.42 | | 4.72 | | 9.84 | |
| O | HOH 6 | 302 | | 3.16 | | 4.08 | | 10.62 | |
| W5 | WAT | 5 | 3 | 3.12 | 0.69 | 4.35 | 0.33 | 10.23 | 0.39 |
| O | HOH 3 | 194 | | 2.39 | | 6.87 | | 10.29 | |
| O | HOH 4 | 220 | | 2.39 | | 7.13 | | 10.16 | |
| O | HOH 6 | 208 | | 2.21 | | 6.90 | | 10.19 | |
| W7 | WAT | 7 | 3 | 2.33 | 0.11 | 6.97 | 0.14 | 10.21 | 0.07 |
| O | HOH 3 | 238 | | 2.04 | | 3.26 | | 9.48 | |
| O | HOH 6 | 301 | | 0.72 | | 3.27 | | 8.67 | |
| W9 | WAT | 9 | 2 | 1.38 | 0.94 | 3.27 | 0.01 | 9.07 | 0.57 |
| O | HOH 3 | 255 | | −2.28 | | −11.29 | | 15.13 | |
| O | HOH 4 | 493 | | −2.82 | | −10.95 | | 15.67 | |
| O | HOH 6 | 266 | | −2.62 | | −12.63 | | 15.07 | |
| W10 | WAT | 10 | 3 | −2.58 | 0.27 | −11.63 | 0.89 | 15.29 | 0.33 |

TABLE 9A
Pharmacofamily 7 Subset

| | | | rmsd from |
|---|---|---|---|
| Molecule # | pdb | type | Family Ave. |
| 1 | 1GET | Glutathione Reductase (E. coli) | 0.34 |
| 2 | 1GRB | Glutathione Reductase (human) | 0.66 |
| 3 | 2NPX | NADH Peroxidase (strep faecalis) | 0.82 |
| 4 | 1TDF | Thioredoxin Reductase (E. coli) | 0.89 |
| 5 | 1TYP | Trypanothione Reductase | 2.17* |
| | | (Crithidia fasciculata) | |

*NAD(P) is in an inactive conformation

TABLE 9B
Polypeptide and Solvent Interactors
(average coordinates)

| | | | | | | | | |
|---|---|---|---|---|---|---|---|---|
| | residue- | | | | | | | |
| atom name | mol. # | total | x | σx | y | σy | z | σz |

Acceptors

| | | | | | | | | |
|---|---|---|---|---|---|---|---|---|
| A11 | ACC | 4 | −3.74 | 0.43 | 4.39 | 1.20 | 14.96 | 0.59 |
| A12 | ACC | 2 | −4.46 | 0.14 | 6.91 | 0.01 | 13.10 | 0.51 |
| A21 | ACC | 3 | −7.67 | 0.40 | −0.28 | 0.63 | 6.97 | 0.49 |
| A27 | ACC | 5 | −6.51 | 0.79 | 8.70 | 0.33 | 10.16 | 0.42 |
| A37 | ACC | 1 | 9.32 | — | 1.02 | — | 6.96 | — |
| A38 | ACC | 1 | 8.04 | — | 2.39 | — | 7.96 | — |
| A43 (D46) | ACC | 1 | −1.72 | — | 2.70 | — | 6.02 | — |
| Donors | | | | | | | | |
| D8 | DON | 5 | 0.53 | 0.17 | 4.12 | 0.23 | 9.87 | 0.65 |
| D10 | DON | 4 | −0.29 | 0.12 | 2.72 | 0.33 | 12.17 | 0.28 |
| D13 | DON | 4 | 11.13 | 0.14 | −1.28 | 0.24 | 5.56 | 0.39 |
| D14 | DON | 4 | 10.96 | 0.24 | −3.44 | 0.24 | 4.80 | 0.45 |
| D15 | DON | 4 | 9.51 | 0.04 | −1.85 | 0.43 | 4.07 | 0.31 |
| D18 | DON | 3 | 8.97 | 1.77 | 3.01 | 1.32 | 1.85 | 0.48 |
| D23 | DON | 5 | 2.38 | 0.54 | −3.84 | 0.13 | 9.65 | 0.30 |
| D46 (A43) | DON | 1 | −1.72 | — | 2.70 | — | 6.02 | — |
| D58 | DON | 1 | 3.70 | — | 2.30 | — | 3.85 | — |
| D62 | DON | 1 | −5.70 | | 2.24 | — | 2.88 | — |
| Waters | | | | | | | | |
| W2 | WAT | 3 | 0.36 | 0.44 | −3.68 | 0.38 | 12.46 | 0.18 |
| W4 | WAT | 4 | 2.93 | 0.16 | 1.13 | 0.26 | 10.91 | 0.18 |
| W6 | WAT | 5 | −9.38 | 0.47 | 6.86 | 0.35 | 8.83 | 0.85 |
| W10 | WAT | 2 | 0.45 | 0.22 | 3.40 | 0.19 | 5.75 | 0.60 |
| W13 | WAT | 3 | −6.28 | 0.08 | −3.16 | 0.26 | 9.68 | 0.49 |

TABLE 9C
NAD(P) Conformer Model

| Atom name | total | x | σx | y | σy | z | σz |
|---|---|---|---|---|---|---|---|
| PA | 5 | 0.93 | 0.13 | −0.09 | 0.32 | 6.93 | 0.27 |
| O1A | 5 | 0.14 | 0.09 | 1.08 | 0.42 | 6.77 | 0.65 |
| O2A | 5 | 1.08 | 0.29 | −1.04 | 0.52 | 5.87 | 0.08 |
| O5′A | 5 | 2.38 | 0.11 | 0.41 | 0.17 | 7.37 | 0.16 |
| C5′A | 5 | 3.43 | 0.24 | −0.49 | 0.18 | 7.71 | 0.15 |
| C4′A | 5 | 4.73 | 0.18 | 0.09 | 0.26 | 7.34 | 0.36 |
| O4′A | 5 | 5.80 | 0.27 | −0.54 | 0.45 | 7.99 | 0.17 |
| O3′A | 5 | 5.07 | 0.14 | −0.04 | 0.62 | 5.96 | 0.38 |
| O3′A | 5 | 4.90 | 0.67 | 0.84 | 0.92 | 5.36 | 0.96 |
| O2′A | 5 | 6.35 | 0.42 | −0.33 | 0.34 | 5.72 | 0.24 |
| O2′A | 5 | 6.88 | 0.18 | 0.71 | 0.74 | 5.16 | 0.35 |
| C1′A | 5 | 6.90 | 0.27 | −0.63 | 0.31 | 7.08 | 0.22 |
| N9A | 5 | 7.56 | 0.16 | −1.93 | 0.24 | 7.16 | 0.17 |
| C8A | 5 | 7.19 | 0.18 | −3.11 | 0.27 | 6.55 | 0.20 |
| N7A | 5 | 7.98 | 0.18 | −4.12 | 0.22 | 6.87 | 0.22 |
| C5A | 5 | 8.90 | 0.17 | −3.57 | 0.15 | 7.72 | 0.19 |
| C6A | 5 | 10.00 | 0.19 | −4.16 | 0.07 | 8.39 | 0.21 |
| N6A | 5 | 10.34 | 0.27 | −5.42 | 0.05 | 8.23 | 0.27 |
| N1A | 5 | 10.72 | 0.16 | −3.34 | 0.07 | 9.17 | 0.23 |
| C2A | 5 | 10.42 | 0.10 | −2.04 | 0.11 | 9.27 | 0.21 |
| N3A | 5 | 9.45 | 0.10 | −1.39 | 0.13 | 8.66 | 0.19 |
| C4A | 5 | 8.68 | 0.13 | −2.21 | 0.16 | 7.90 | 0.17 |
| O3 | 5 | 0.38 | 0.10 | −0.91 | 0.20 | 8.17 | 0.20 |
| PN | 5 | −0.15 | 0.14 | −0.48 | 0.48 | 9.57 | 0.41 |
| O2N | 5 | 0.14 | 0.49 | 0.83 | 0.44 | 9.75 | 0.95 |
| O1N | 5 | 0.30 | 0.16 | −1.45 | 1.05 | 10.42 | 0.24 |
| O5′N | 5 | −1.69 | 0.19 | −0.59 | 0.27 | 9.56 | 0.17 |
| C5′N | 5 | −2.47 | 0.06 | −1.57 | 0.23 | 8.85 | 0.37 |
| C4′N | 5 | −3.70 | 0.14 | −0.94 | 0.26 | 8.22 | 0.15 |
| O4′N | 5 | −4.71 | 0.05 | −0.62 | 0.08 | 9.19 | 0.03 |
| C3′N | 5 | −3.46 | 0.22 | 0.35 | 0.46 | 7.53 | 0.17 |
| O3′N | 5 | −3.17 | 0.71 | 0.29 | 0.62 | 6.28 | 0.17 |
| C2′N | 5 | −4.65 | 0.52 | 1.11 | 0.18 | 7.65 | 0.18 |
| O2′N | 5 | −5.28 | 0.75 | 0.98 | 0.55 | 6.52 | 0.28 |
| C1′N | 5 | −5.38 | 0.18 | 0.60 | 0.07 | 8.82 | 0.16 |
| N1N | 5 | −5.34 | 0.08 | 1.60 | 0.06 | 9.91 | 0.18 |
| C2N | 5 | −5.97 | 0.21 | 2.80 | 0.05 | 9.75 | 0.25 |
| C3N | 5 | −5.93 | 0.17 | 3.83 | 0.08 | 10.68 | 0.26 |
| C7N | 5 | −6.64 | 0.26 | 5.15 | 0.08 | 10.42 | 0.36 |
| O7N | 5 | −7.25 | 0.57 | 5.32 | 0.37 | 9.88 | 1.12 |
| N7N | 5 | −6.58 | 0.34 | 6.07 | 0.28 | 10.81 | 0.74 |
| C4N | 5 | −5.15 | 0.02 | 3.67 | 0.21 | 11.82 | 0.22 |
| C5N | 5 | −4.45 | 0.21 | 2.46 | 0.27 | 11.97 | 0.23 |
| C6N | 5 | −4.58 | 0.19 | 1.45 | 0.20 | 11.02 | 0.20 |
| P2′ | 3 | 8.26 | 0.32 | 1.61 | 0.37 | 4.55 | 0.21 |
| OP1 | 3 | 8.14 | 0.53 | 1.73 | 0.94 | 3.60 | 0.75 |
| OP2 | 3 | 9.03 | 0.56 | 1.00 | 0.50 | 4.62 | 1.13 |
| OP3 | 3 | 8.62 | 0.79 | 2.41 | 1.40 | 4.94 | 0.68 |

TABLE 9D
Polypeptide and Solvent Interactors

| atom name | residue-mol # | residue # | total | x | σx | y | σy | z | σz |
|---|---|---|---|---|---|---|---|---|---|
| Acceptors | | | | | | | | | |
| OE1 | GLU 1 | 181 | | −3.88 | | 5.25 | | 14.75 | |
| OE1 | GLU 2 | 201 | | −4.15 | | 5.48 | | 14.38 | |
| OE1 | GLU 3 | 163 | | −3.79 | | 3.89 | | 15.77 | |
| OE1 | GLU 4 | 159 | | −3.14 | | 2.93 | | 14.95 | |
| A11 | ACC | 11 | 4 | −3.74 | 0.43 | 4.39 | 1.20 | 14.96 | 0.59 |
| OE2 | GLU 1 | 181 | | −4.37 | | 6.90 | | 13.45 | |
| OE2 | GLU 2 | 201 | | −4.56 | | 6.92 | | 12.74 | |
| A12 | ACC | 12 | 2 | −4.46 | 0.14 | 6.91 | 0.01 | 13.10 | 0.51 |
| O | GLU 1 | 309 | | −8.06 | | 0.25 | | | 7.52 |
| O | LEU 2 | 337 | | −7.71 | | −0.11 | | 6.85 | |
| O | ALA 3 | 297 | | −7.26 | | −0.97 | | 6.55 | |
| A21 | ACC | 21 | 3 | −7.67 | 0.40 | −0.28 | 0.63 | 6.97 | 0.49 |
| OE2 | GLU 1 | 309 | | −4.36 | | −3.87 | | 5.45 | |
| A23 | ACC | 23 | 1 | −4.36 | | −3.87 | | 5.45 | |
| O | VAL 1 | 342 | | −7.20 | | 8.83 | | 10.41 | |
| O | VAL 2 | 370 | | −6.94 | | 8.48 | | 9.46 | |
| O | GLY 3 | 328 | | −6.79 | | 9.23 | | 10.09 | |
| OE2 | GLU 4 | 183 | | −5.19 | | 8.47 | | 10.50 | |
| O | ALA 5 | 365 | | −6.46 | | 8.51 | | 10.35 | |
| A27 | ACC | 27 | 5 | −6.51 | 0.79 | 8.70 | 0.33 | 10.16 | 0.42 |
| OD1 | ASP 3 | 179 | | 9.32 | | 1.02 | | 6.96 | |
| A37 | ACC | 37 | 1 | 9.32 | | 1.02 | | 6.96 | |
| OD2 | ASP 3 | 179 | | 8.04 | | 2.39 | | 7.96 | |
| A38 | ACC | 38 | 1 | 8.04 | | 2.39 | | 7.96 | |
| OH | TYR 3 | 188 | | −1.72 | | 2.70 | | 6.02 | |
| A43 | ACC | 43 | 1 | −1.72 | | 2.70 | | 6.02 | |
| Donors | | | | | | | | | |
| N | TYR 1 | 177 | | 0.42 | | 4.12 | | 9.29 | |
| N | TYR 2 | 197 | | 0.54 | | 3.95 | | 9.16 | |
| N | TYR 3 | 159 | | 0.39 | | 3.86 | | 9.94 | |
| N | ASN 4 | 155 | | 0.81 | | 4.22 | | 10.27 | |
| N | TYR 5 | 198 | | 0.50 | | 4.45 | | 10.69 | |
| D8 | DON | 8 | 5 | 0.53 | 0.17 | 4.12 | 0.23 | 9.87 | 0.65 |
| N | ILE 1 | 178 | | −0.30 | | 3.00 | | 11.99 | |
| N | ILE 2 | 198 | | −0.19 | | 3.01 | | 11.87 | |
| N | ILE 3 | 160 | | −0.46 | | 2.46 | | 12.45 | |
| N | THR 4 | 156 | | −0.21 | | 2.41 | | 12.37 | |
| D10 | DON | 10 | 4 | −0.29 | 0.12 | 2.72 | 0.33 | 12.17 | 0.28 |
| NE | ARG 1 | 198 | | 10.97 | | −1.63 | | 5.67 | |
| NE | ARG 2 | 218 | | 11.27 | | −1.15 | | 5.31 | |
| NE | ARG 4 | 176 | | 11.22 | | −1.28 | | 5.21 | |
| NE | ARG 5 | 222 | | 11.04 | | −3.80 | | 6.07 | |
| D13 | DON | 13 | 4 | 11.13 | 0.14 | −1.28 | 0.24 | 5.56 | 0.39 |
| NH1 | ARG 1 | 198 | | 11.24 | | −3.80 | | 4.93 | |
| NH1 | ARG 2 | 218 | | 10.89 | | −3.37 | | 4.77 | |
| NH1 | ARG 4 | 176 | | 10.67 | | −3.32 | | 4.21 | |
| NH1 | ARG 5 | 222 | | 11.05 | | −3.27 | | 5.30 | |
| D14 | DON | 14 | 4 | 10.96 | 0.24 | −3.44 | 0.24 | 4.80 | 0.45 |
| NH2 | ARG 1 | 198 | | 9.54 | | −2.45 | | 4.11 | |
| VAL 1 | ARG 2 | 218 | | 9.46 | | −1.77 | | 4.00 | |
| NH2 | ARG 4 | 176 | | 9.50 | | −1.43 | | 3.70 | |
| NH2 | ARG 5 | 222 | | 9.55 | | −1.74 | | 4.46 | |
| D15 | DON | 15 | 4 | 9.51 | 0.04 | −1.85 | 0.43 | 4.07 | 0.31 |
| NE | ARG 4 | 177 | | 10.99 | | 4.32 | | 2.39 | |
| NH1 | ARG 1 | 204 | | 8.17 | | 3.03 | | 1.71 | |
| NH1 | ARG 5 | 228 | | 7.75 | | 1.68 | | 1.45 | |
| D18 | DON | 18 | 3 | 8.97 | 1.77 | 3.01 | 1.32 | 1.85 | 0.48 |
| N | GLY 1 | 262 | | 2.72 | | −3.76 | | 9.55 | |
| N | GLY 2 | 290 | | 2.62 | | −3.74 | | 9.51 | |
| N | GLY 3 | 243 | | 2.38 | | −4.07 | | 9.32 | |
| N | GLY 4 | 244 | | 1.45 | | −3.80 | | 10.09 | |
| N | GLY 5 | 286 | | 2.74 | | −3.85 | | 9.80 | |
| D23 | DON | 23 | 5 | 2.38 | 0.54 | −3.84 | 0.13 | 9.65 | 0.30 |
| OH | TYR 3 | 188 | | −1.72 | | 2.70 | | 6.02 | |
| D46 | DON | 46 | 1 | −1.72 | | 2.70 | | 6.02 | |
| NH1 | ARG 4 | 181 | | 3.70 | | 2.30 | | 3.85 | |
| D58 | DON | 58 | 1 | 3.70 | | 2.30 | | 3.85 | |
| ND2 | ASN 4 | 260 | | −5.70 | | 2.24 | | 2.88 | |
| D62 | DON | 62 | 1 | −5.70 | | 2.24 | | 2.88 | |
| Waters | | | | | | | | | |
| O | HOH 1 | 35 | | 0.68 | | −3.50 | | 12.51 | |
| O | HOH 2 | 511 | | 0.54 | | −3.42 | | 12.61 | |
| O | HOH 3 | 461 | | −0.15 | | −4.12 | | 12.26 | |
| W2 | WAT | 2 | 3 | 0.36 | 0.44 | −3.38 | 0.38 | 12.46 | 0.18 |
| O | HOH 1 | 70 | | 2.74 | | 1.12 | | 10.80 | |
| O | HOH 2 | 524 | | 3.09 | | 1.48 | | 10.72 | |
| O | HOH 3 | 901 | | 2.86 | | 1.06 | | 11.09 | |
| O | HOH 4 | 618 | | 3.03 | | 0.85 | | 11.05 | |
| W4 | WAT | 4 | 4 | 2.93 | 0.16 | 1.13 | 0.26 | 10.91 | 0.18 |
| O | HOH 1 | 115 | | −9.62 | | 7.01 | | 9.04 | |
| O | HOH 2 | 514 | | −9.26 | | 6.65 | | 7.93 | |
| O | HOH 3 | 499 | | −8.71 | | 7.08 | | 8.17 | |
| O | HOH 4 | 861 | | −9.99 | | 6.36 | | 10.10 | |
| O | HOH 5 | 121 | | −9.93 | | 7.20 | | 8.93 | |
| W6 | WAT | 6 | 5 | −9.38 | 0.47 | 6.86 | 0.35 | 8.83 | 0.85 |
| O | HOH 1 | 171 | | 0.30 | | 3.54 | | 6.18 | |
| O | HOH 2 | 984 | | 0.61 | | 3.27 | | 5.33 | |
| W10 | WAT | 10 | 2 | 0.45 | 0.22 | 3.40 | 0.19 | 5.75 | 0.60 |
| O | HOH 1 | 250 | | −6.35 | | −3.18 | | 10.09 | |
| O | HOH 2 | 500 | | −6.31 | | −2.89 | | 9.82 | |
| O | HOH 3 | 467 | | −6.19 | | −3.41 | | 9.14 | |
| W13 | WAT | 13 | 3 | −6.28 | 0.08 | −3.16 | 0.26 | 9.68 | 0.49 |

TABLE 10A
Pharmacofamily 8 Subset

| | | | rmsd from |
|---|---|---|---|
| Molecule # | pdb | type | family ave. |
| 1 | 1QGA | Ferrodoxin Reductase (pea) | 0.61 |
| 2 | P450 ′ | P450 reductase (rat) | 0.35 |

TABLE 10B
Polypeptide and Solvent Interactors (average coordinates)

| atom name | residue-mol. # | total | x | σx | y | σy | z | σz |
|---|---|---|---|---|---|---|---|---|
| Acceptors | | | | | | | | |
| A2 | ACC | 2 | 0.63 | 0.38 | −6.60 | 0.21 | −7.09 | 0.16 |
| A8 | ACC | 2 | −2.87 | 0.25 | −3.55 | 0.64 | −0.51 | 0.02 |
| A11 | ACC | 2 | −4.28 | 0.30 | 8.10 | 0.34 | 3.52 | 0.33 |
| A14 | ACC | 2 | −7.58 | 0.10 | 8.62 | 0.24 | 3.69 | 0.19 |
| A18 | ACC | 2 | −12.53 | 0.11 | 8.89 | 0.59 | 0.72 | 0.62 |
| A21 | ACC | 2 | −8.28 | 0.08 | 9.45 | 0.25 | −6.25 | 0.84 |
| A23 | ACC | 2 | −1.15 | 0.00 | −2.54 | 0.21 | −7.56 | 0.09 |
| A29 | ACC | 2 | −1.63 | 0.84 | −6.66 | 0.42 | −10.70 | 0.06 |
| A31 | ACC | 2 | −7.49 | 0.70 | −5.59 | 0.66 | −9.88 | 0.66 |
| A32 | ACC | 1 | −8.95 | — | −3.74 | — | −4.78 | — |
| Donors | | | | | | | | |
| D2 | DON | 2 | 0.63 | 0.38 | −6.60 | 0.21 | −7.09 | 0.16 |
| D4 | DON | 2 | −6.69 | 0.23 | −1.87 | 0.78 | 5.73 | 0.27 |
| D8 | DON | 2 | −1.98 | 0.25 | −0.80 | 0.53 | −0.07 | 0.05 |
| D9 | DON | 2 | −2.87 | 0.25 | −3.55 | 0.64 | −0.51 | 0.02 |
| D15 | DON | 2 | −7.58 | 0.10 | 8.62 | 0.24 | 3.69 | 0.19 |
| D18 | DON | 2 | −10.73 | 0.10 | 5.15 | 0.70 | 6.85 | 0.21 |
| D21 | DON | 2 | −12.39 | 0.55 | 8.95 | 0.83 | 4.42 | 0.46 |
| D23 | DON | 2 | −12.53 | 0.11 | 8.89 | 0.59 | 0.72 | 0.62 |
| D26 | DON | 2 | −10.08 | 0.70 | 9.97 | 0.39 | −5.61 | 0.35 |

TABLE 10C
NAD (P) Conformer Model

| atom name | number | x | σx | y | σy | z | σz |
|---|---|---|---|---|---|---|---|
| PA | 2 | −6.90 | 0.19 | 1.29 | 0.01 | 2.19 | 0.44 |
| O1A | 2 | −8.23 | 0.13 | 0.84 | 0.28 | 2.29 | 1.01 |
| O2A | 2 | −6.22 | 0.68 | 1.25 | 0.00 | 3.45 | 0.19 |
| O5′A | 2 | −6.94 | 0.05 | 2.74 | 0.01 | 1.67 | 0.46 |
| C5′A | 2 | −5.96 | 0.32 | 3.31 | 0.21 | 0.99 | 0.16 |
| C4′A | 2 | −6.21 | 0.28 | 4.77 | 0.19 | 0.81 | 0.08 |
| O4′A | 2 | −7.07 | 0.21 | 4.93 | 0.07 | −0.33 | 0.12 |
| C3′A | 2 | −6.95 | 0.32 | 5.45 | 0.19 | 1.99 | 0.09 |
| O3′A | 2 | −6.38 | 0.22 | 6.74 | 0.20 | 2.25 | 0.09 |
| C2′A | 2 | −8.36 | 0.28 | 5.60 | 0.08 | 1.51 | 0.12 |
| O2′A | 2 | −9.02 | 0.09 | 6.71 | 0.01 | 2.15 | 0.10 |
| C1′A | 2 | −8.10 | 0.23 | 5.82 | 0.11 | 0.05 | 0.07 |
| N9A | 2 | −9.26 | 0.18 | 5.67 | 0.07 | −0.81 | 0.09 |
| C8A | 2 | −10.48 | 0.15 | 5.08 | 0.02 | −0.58 | 0.05 |
| N7A | 2 | −11.35 | 0.01 | 5.15 | 0.09 | −1.61 | 0.14 |
| C5A | 2 | −10.62 | 0.05 | 5.84 | 0.01 | −2.55 | 0.11 |
| C6A | 2 | −10.98 | 0.07 | 6.27 | 0.00 | −3.84 | 0.10 |
| N6A | 2 | −12.17 | 0.06 | 6.02 | 0.00 | −4.36 | 0.08 |
| N1A | 2 | −10.08 | 0.13 | 6.95 | 0.04 | −4.59 | 0.09 |
| C2A | 2 | −8.88 | 0.12 | 7.22 | 0.07 | −4.10 | 0.04 |
| N3A | 2 | −8.46 | 0.02 | 6.87 | 0.15 | −2.90 | 0.02 |
| C4A | 2 | −9.35 | 0.07 | 6.17 | 0.04 | −2.06 | 0.07 |
| O3 | 2 | −6.11 | 0.32 | 0.30 | 0.20 | 1.21 | 0.13 |
| PN | 2 | −5.73 | 0.14 | −1.29 | 0.24 | 1.48 | 0.01 |
| O1N | 2 | −6.50 | 0.06 | −1.63 | 0.42 | 2.69 | 0.13 |
| O2N | 2 | −4.30 | 0.14 | −1.48 | 0.06 | 1.62 | 0.06 |
| O5′N | 2 | −6.26 | 0.37 | −2.13 | 0.26 | 0.26 | 0.06 |
| C5′N | 2 | −5.67 | 0.29 | −2.09 | 0.15 | −1.01 | 0.07 |
| C4′N | 2 | −6.63 | 0.26 | −2.81 | 0.33 | −1.93 | 0.11 |
| O4′N | 2 | −6.11 | 0.28 | −2.90 | 0.27 | −3.27 | 0.09 |
| C3′N | 2 | −6.95 | 0.06 | −4.24 | 0.38 | −1.45 | 0.14 |
| O3′N | 2 | −8.35 | 0.03 | −4.47 | 0.60 | −1.50 | 0.32 |
| C2′N | 2 | −6.22 | 0.01 | −5.16 | 0.30 | −2.41 | 0.06 |
| O2′N | 2 | −7.01 | 0.15 | −6.29 | 0.42 | −2.74 | 0.07 |
| C1′N | 2 | −5.90 | 0.11 | −4.29 | 0.22 | −3.62 | 0.04 |
| NN1 | 2 | −4.55 | 0.05 | −4.52 | 0.01 | −4.21 | 0.01 |
| C2N | 2 | −4.50 | 0.03 | −5.07 | 0.06 | −5.47 | 0.05 |
| C3N | 2 | −3.29 | 0.08 | −5.32 | 0.10 | −6.13 | 0.01 |
| C7N | 2 | −3.24 | 0.24 | −5.90 | 0.02 | −7.52 | 0.03 |
| O7N | 2 | −3.24 | 1.75 | −6.01 | 0.02 | −8.11 | 0.03 |
| NN7 | 2 | −3.18 | 1.32 | −6.31 | 0.10 | −8.11 | 0.04 |
| C4N | 2 | −2.09 | 0.01 | −5.00 | 0.39 | −5.44 | 0.02 |
| C5N | 2 | −2.15 | 0.06 | −4.44 | 0.46 | −4.14 | 0.07 |
| C6N | 2 | −3.40 | 0.11 | −4.21 | 0.25 | −3.54 | 0.08 |
| P2′ | 2 | −10.21 | 0.02 | 6.47 | 0.10 | 3.22 | 0.06 |
| OP1 | 2 | −10.72 | 1.21 | 5.88 | 0.71 | 3.20 | 1.26 |
| OP2 | 2 | −10.31 | 0.01 | 7.62 | 0.12 | 4.24 | 0.11 |
| OP3 | 2 | −10.73 | 1.02 | 5.69 | 1.01 | 3.24 | 0.93 |

TABLE 10D
Polypeptide and Solvent Interactors

| | residue- | | | | | | | | |
|---|---|---|---|---|---|---|---|---|---|
| atom name | mol. # | residue # | total | x | σx | y | σy | z | σz |
| Acceptors | | | | | | | | | |
| OG | SER 1 | 90 | | 0.366 | | −6.74 | | −6.97 | |
| OG | SER 2 | 457 | | 0.899 | | −6.45 | | −7.20 | |
| A2 | ACC | 2 | 2 | 0.633 | 0.38 | −6.60 | 0.21 | −7.09 | 0.16 |
| OG1 | THR 1 | 166 | | −2.694 | | −4.00 | | −0.53 | |
| OG1 | THR 2 | 535 | | −3.041 | | −3.09 | | −0.50 | |
| A8 | ACC | 8 | 2 | −2.867 | 0.25 | −3.55 | 0.64 | −0.51 | 0.02 |
| O | VAL 1 | 198 | | −4.071 | | 7.86 | | 3.28 | |
| O | CYS 2 | 566 | | −4.494 | | 8.34 | | 3.75 | |
| A11 | ACC | 11 | 2 | −4.282 | 0.30 | 8.10 | 0.34 | 3.52 | 0.33 |
| OG | SER 1 | 228 | | −7.649 | | 8.79 | | 3.55 | |
| OG | SER 2 | 596 | | −7.509 | | 8.45 | | 3.83 | |
| A14 | ACC | 14 | 2 | −7.579 | 0.10 | 8.62 | 0.24 | 3.69 | 0.19 |
| OH | TYR 1 | 240 | | −12.45 | | 9.30 | | 1.16 | |
| OH | TYR 2 | 604 | | −12.61 | | 8.47 | | 0.29 | |
| A18 | ACC | 18 | 2 | −12.53 | 0.11 | 8.89 | 0.59 | 0.72 | 0.62 |
| OE1 | GLN 1 | 242 | | −8.226 | | 9.28 | | −6.85 | |
| OE1 | GLN 2 | 606 | | −8.34 | | 9.63 | | −5.65 | |
| A21 | ACC | 21 | 2 | −8.283 | 0.08 | 9.45 | 0.25 | −6.25 | 0.84 |
| SG | CYS 1 | 266 | | −1.15 | | −2.68 | | −7.63 | |
| SG | CYS 2 | 630 | | −1.148 | | −2.39 | | −7.50 | |
| A23 | ACC | 23 | 2 | −1.149 | 0.00 | −2.54 | 0.21 | −7.56 | 0.09 |
| OE1 | GLU 1 | 306 | | −1.033 | | −6.96 | | −10.66 | |
| OD1 | ASP 2 | 675 | | −2.227 | | −6.36 | | −10.74 | |
| A29 | ACC | 29 | 2 | −1.63 | 0.84 | −6.66 | 0.42 | −10.70 | 0.06 |
| O | VAL 1 | 307 | | −7.979 | | −5.12 | | −9.41 | |
| O | VAL 2 | 676 | | −6.991 | | −6.05 | | −10.34 | |
| A31 | ACC | 31 | 2 | −7.485 | 0.70 | −5.59 | 0.66 | −9.88 | 0.66 |
| O | TRP 1 | 308 | | −8.949 | | −3.74 | | −4.78 | |
| A32 | ACC | 32 | 1 | −8.949 | | −3.74 | | −4.78 | |
| Donors | | | | | | | | | |
| OG | SER 1 | 90 | | 0.366 | | −6.74 | | −6.97 | |
| OG | SER 2 | 457 | | 0.899 | | −6.45 | | −7.20 | |
| D2 | DON | 2 | 2 | 0.633 | 0.38 | −6.60 | 0.21 | −7.09 | 0.16 |
| NZ | LYS 1 | 110 | | −6.847 | | −2.42 | | 5.92 | |
| NH1 | ARG 2 | 298 | | −6.526 | | −1.32 | | 5.54 | |
| D4 | DON | 4 | 2 | −6.687 | 0.23 | −1.87 | 0.78 | 5.73 | 0.27 |
| N | THR 1 | 166 | | −1.805 | | −1.18 | | −0.10 | |
| N | THR 2 | 535 | | −2.152 | | −0.42 | | −0.03 | |
| D8 | DON | 8 | 2 | −1.978 | 0.25 | −0.80 | 0.53 | −0.07 | 0.05 |
| OG1 | THR 1 | 166 | | −2.694 | | −4.00 | | −0.53 | |
| OG1 | THR 2 | 535 | | −3.041 | | −3.09 | | −0.50 | |
| D9 | DON | 9 | 2 | −2.867 | 0.25 | −3.55 | 0.64 | −0.51 | 0.02 |
| OG | SER 1 | 228 | | −7.649 | | 8.79 | | 3.55 | |
| OG | SER 2 | 596 | | −7.509 | | 8.45 | | 3.83 | |
| D15 | DON | 15 | 2 | −7.579 | 0.10 | 8.62 | 0.24 | 3.69 | 0.19 |
| NH1 | ARG 1 | 229 | | −10.66 | | 5.64 | | 7.00 | |
| NH2 | ARG 2 | 597 | | −10.81 | | 4.65 | | 6.71 | |
| D18 | DON | 18 | 2 | −10.73 | 0.10 | 5.15 | 0.70 | 6.85 | 0.21 |
| NZ | LYS 1 | 238 | | −12 | | 9.53 | | 4.09 | |
| NZ | LYS 2 | 602 | | −12.78 | | 8.36 | | 4.75 | |
| D21 | DON | 21 | 2 | −12.39 | 0.55 | 8.95 | 0.83 | 4.42 | 0.46 |
| OH | TYR 1 | 240 | | −12.45 | | 9.30 | | 1.16 | |
| OH | TYR 2 | 604 | | −12.61 | | 8.47 | | 0.29 | |
| D23 | DON | 23 | 2 | −12.53 | 0.11 | 8.89 | 0.59 | 0.72 | 0.62 |
| NE2 | GLN 1 | 242 | | −9.587 | | 10.24 | | −5.36 | |
| NE2 | GLN 2 | 606 | | −10.58 | | 9.70 | | −5.85 | |
| D26 | DON | 26 | 2 | −10.08 | 0.70 | 9.97 | 0.39 | −5.61 | 0.35 |

Coordinates for the conformer and pharmacophore models and data used in their construction is presented in Tables 3-10 above. Part A of each Table lists subset of structures used in constructing the model including molecule numbers for cross-referencing between parts A-C, the PDB accession number, the name of the polypeptide, and the RMSD from the pharmacocluster average. Part B of each Table lists the average coordinates for heteroatoms and waters of the pharmacophore model and includes the atom name (cross referenced to part D), designation of interaction (“ACC,” acceptor; “DON,” donor; and “WAT,” water), total number of atoms included in the calculation of the average, and X, Y, Z coordinates with respective standard deviations (σ). Part C of each Table lists the coordinates of the conformer model using the atom designations of FIG. 2 and X, Y, Z coordinates with respective standard deviations (σ). Part D of each Table lists the coordinates for interacting molecules used to determine the pharmacophore model including the atom name, residue molecule # (which identifies the residue type and molecule number cross-referenced to Part A), residue number from the PDB structure, total number of atoms summed for the average coordinates, and X, Y, Z coordinates with respective standard deviations (σ). The bolded entries in part D correspond to the average values reported in part B. Atom names are identified according to IUPAC recommendations as described for example in Markley et al., Pure and Appl. Chem. 70:117-142 (1998).

Throughout this application various publications have been referenced. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific details are only illustrative of the invention. It is understood that modifications which do not substantially affect the activity of the various embodiments of this invention are also included within the definition of the invention provided herein. Therefore, it should be understood that various modifications can be made without departing from the spirit of the invention. Accordingly, the invention is limited only by the following claims.

What is claimed is:

1. A method for identifying a pharmacocluster, comprising:
(a) determining bound conformations of a ligand bound to different polypeptides; and
(b) clustering two or more bound conformations of said ligand having substantially the same bound conformation, thereby identifying a pharmacocluster.

2. The method of claim 1, wherein substantially the same bound conformation comprises a root mean square deviation of less than 1.1 Å.

3. The method of claim 1, wherein said ligand is selected from the group consisting of adenosine triphosphate, adenosine diphosphate, adenosine monophosphate thiamine (vitamin B₁), riboflavin (vitamin B₂), pyridoximine (vitamin B₆), cobalamin (vitamin B₁₂), pyrophosphate, flavin adenine dinucleotide (FAD); flavin mononucleotide (FMN), pyridoxal phosphate, coenzyme A, ascorbate (vitamin C), niacin, biotin, heme, porphyrin, folate, tetrahydrofolate, guanosine triphosphate, cytidine triphosphate, thymidine triphosphate, uridine triphosphate, retinol (vitamin A), calciferol (vitamin D₂), ubiquinone, ubiquitin, α-tocopherol (vitamin E), farnesyl, geranylgeranyl, pterin, pteridine or S-adenosyl methionine (SAM).

4. The method of claim 1, wherein said ligand comprises a nicotinamide adenine dinucleotide-related molecule.

5. The method of claim 4, wherein said nicotinamide adenine dinucleotide-related molecule is selected from the group consisting of oxidized nicotinamide adenine dinucleotide, reduced nicotinamide adenine dinucleotide, oxidized nicotinamide adenine dinucleotide phosphate, reduced nicotinamide adenine dinucleotide phosphate, and a mimetic thereof.

6. A method for identifying a member of a pharmacocluster, comprising:
(a) determining a bound conformation of a ligand bound to a polypeptide; and
(b) determining a pharmacocluster having substantially the same bound conformation as said bound conformation, thereby identifying said bound conformation of said ligand as a member of said pharmacocluster.

7. The method of claim 6, wherein substantially the same bound conformation comprises a root mean square deviation of less than 1.1 Å.

8. The method of claim 6, wherein said ligand is selected from the group consisting of adenosine triphosphate, adenosine diphosphate, adenosine monophosphate thiamine (vitamin B₁), riboflavin (vitamin B₂), pyridoximine (vitamin B₆), cobalamin (vitamin B₁₂), pyrophosphate, flavin adenine dinucleotide (FAD), flavin mononucleotide (FMN), pyridoxal phosphate, coenzyme A, ascorbate (vitamin C), niacin, biotin, heme, porphyrin, folate, tetrahydrofolate, guanosine triphosphate, cytidine triphosphate, thymidine triphosphate, uridine triphosphate, retinol (vitamin A), calciferol (vitamin D₂), ubiquinone, ubiquitin, α-tocopherol (vitamin E), farnesyl, geranylgeranyl, pterin, pteridine or S-adenosyl methionine (SAM).

9. The method of claim 6, wherein said ligand comprises a nicotinamide adenine dinucleotide-related molecule.

10. The method of claim 9, wherein said nicotinamide adenine dinucleotide-related molecule is selected from the group consisting of oxidized nicotinamide adenine dinucleotide, reduced nicotinamide adenine dinucleotide, oxidized nicotinamide adenine dinucleotide phosphate, reduced nicotinamide adenine dinucleotide phosphate, and a mimetic thereof.

11. A method for identifying a conformation-dependent property of a ligand, comprising:
(a) determining bound conformations of a ligand bound to different polypeptides;
(b) identifying two or more bound conformations of said ligand having substantially the same bound conformation; and
(c) identifying a conformation-dependent property of said bound conformations of said ligand having substantially the same bound conformation, said conformation-dependent property being correlated with said bound conformation of said ligand.

12. The method of claim 11, wherein said conformation-dependent property comprises a spectroscopic signal.

13. The method of claim 11, wherein said conformation-dependent property comprises an NMR signal.

14. The method of claim 13, wherein said NMR signal is selected from the group consisting of chemical shift, J coupling, dipolar coupling, cross-correlation, nuclear spin relaxation, transferred nuclear Overhauser effect, and any combination thereof.

15. The method of claim 11, wherein substantially the same bound conformation comprises a root mean square deviation of less than 1.1 Å.

16. The method of claim 11, wherein said ligand is selected from the group consisting of adenosine triphosphate, adenosine diphosphate, adenosine monophosphate thiamine (vitamin B₁), riboflavin (vitamin B₂), pyridoximine (vitamin B₆), cobalamin (vitamin B₁₂), pyrophosphate, flavin adenine dinucleotide (FAD), flavin mononucleotide (FMN), pyridoxal phosphate, coenzyme A, ascorbate (vitamin C), niacin, biotin, heme, porphyrin, folate, tetrahydrofolate, guanosine triphosphate, cytidine triphosphate, thymidine 10 triphosphate, uridine triphosphate, retinol (vitamin A), calciferol (vitamin D₂), ubiquinone, ubiquitin, α-tocopherol (vitamin E), farnesyl, geranylgeranyl, pterin, pteridine or S-adenosyl methionine (SAM).

17. The method of claim 11, wherein said ligand comprises a nicotinamide adenine dinucleotide-related molecule.

18. The method of claim 17, wherein said nicotinamide adenine dinucleotide-related molecule is selected from the group consisting of oxidized nicotinamide adenine dinucleotide, reduced nicotinamide adenine dinucleotide, oxidized nicotinamide adenine dinucleotide phosphate, reduced nicotinamide adenine dinucleotide phosphate, and a mimetic thereof.

19. A method for identifying polypeptide pharmacofamilies, comprising:
(a) determining bound conformations of a ligand bound to different polypeptides of a polypeptide family; and
(b) identifying two or more bound conformations of said ligand having substantially different bound conformations, thereby identifying at least two polypeptide pharmacofamilies exhibiting binding specificity for said two or more substantially different bound conformations of said ligand.

20. The method of claim 19, wherein said polypeptide pharmacofamily is selected from the group consisting of pharmacofamily 1, pharmacofamily 2, pharmacofamily 3, pharmacofamily 4, pharmacofamily 5, pharmacofamily 6, pharmacofamily 7, and pharmacofamily 8.

21. The method of claim 19, wherein said ligand is selected from the group consisting of adenosine triphosphate, adenosine diphosphate, adenosine monophosphate thiamine (vitamin B₁), riboflavin (vitamin B₂), pyridoximine (vitamin B₆), cobalamin (vitamin B₁₂), pyrophosphate, flavin adenine dinucleotide (FAD), flavin mononucleotide (FMN), pyridoxal phosphate, coenzyme A, ascorbate (vitamin C), niacin, biotin, heme, porphyrin, folate, tetrahydrofolate, guanosine triphosphate, cytidine triphosphate, thymidine triphosphate, uridine triphosphate, retinol (vitamin A), calciferol (vitamin D₂), ubiquinone, ubiquitin, α-tocopherol (vitamin E), farnesyl, geranylgeranyl, pterin, pteridine or S-adenosyl methionine (SAM).

22. The method of claim 19, wherein said ligand comprises a nicotinamide adenine dinucleotide-related molecule.

23. The method of claim 22, wherein said nicotinamide adenine dinucleotide-related molecule is selected from the group consisting of oxidized nicotinamide adenine dinucleotide, reduced nicotinamide adenine dinucleotide, oxidized nicotinamide adenine dinucleotide phosphate, reduced nicotinamide adenine dinucleotide phosphate, and a mimetic thereof.

24. A method for identifying a member of a polypeptide pharmacofamily, comprising:
(a) determining a conformation-dependent property of a ligand bound to a polypeptide; and
(b) determining a pharmacocluster having substantially the same conformation-dependent property as said conformation-dependent property determined for said bound ligand, wherein a polypeptide pharmacofamily binds said ligand in a conformation of said pharmacocluster, thereby identifying said polypeptide as a member of said polypeptide pharmacofamily.

25. The method of claim 24, wherein said conformation-dependent property comprises a spectroscopic signal.

26. The method of claim 24, wherein said conformation-dependent property comprises an NMR signal.

27. The method of claim 26, wherein said NMR signal is selected from the group consisting of chemical shift, J coupling, dipolar coupling, cross-correlation, nuclear spin relaxation, transferred nuclear Overhauser effect, and any combination thereof.

28. The method of claim 24, wherein said ligand is selected from the group consisting of adenosine triphosphate, adenosine diphosphate, adenosine monophosphate thiamine (vitamin B₁), riboflavin (vitamin B₂), pyridoximine (vitamin B₆), cobalamin (vitamin B₁₂), pyrophosphate, flavin adenine dinucleotide (FAD), flavin mononucleotide (FMN), pyridoxal phosphate, coenzyme A, ascorbate (vitamin C), niacin, biotin, heme, porphyrin, folate, tetrahydrofolate, guanosine triphosphate, cytidine triphosphate, thymidine triphosphate, uridine triphosphate, retinol (vitamin A), calciferol (vitamin D₂), ubiquinone, ubiquitin, α-tocopherol (vitamin E), farnesyl, geranylgeranyl, pterin, pteridine or S-adenosyl methionine (SAM).

29. The method of claim 24, wherein said ligand is a nicotinamide adenine dinucleotide-related molecule.

30. The method of claim 29, wherein said nicotinamide adenine dinucleotide-related molecule is selected from the group consisting of oxidized nicotinamide adenine dinucleotide, reduced nicotinamide adenine dinucleotide, oxidized nicotinamide adenine dinucleotide phosphate, reduced nicotinamide adenine dinucleotide phosphate, and a mimetic thereof.

31. The method of claim 24, wherein said ligand is a adenosine phosphate-related molecule.

32. The method of claim 31, wherein said adenosine phosphate-related molecule is selected from the group consisting of adenosine triphosphate, adenosine diphosphate, adenosine monophosphate, and a mimetic thereof.

33. A method of modeling the three dimensional structure of a polypeptide, comprising the method of claim 24 followed by the step of:
(c) modeling the three dimensional structure of said polypeptide according to a structural model of said second member of said polypeptide pharmacofamily.

34. A method for constructing a ligand conformer model, comprising determining an average structure of the bound conformations of a ligand in a pharmacocluster.

35. The method of claim 34, wherein said ligand comprises a nicotinamide adenine dinucleotide-related molecule.

36. The method of claim 35, wherein said nicotinamide adenine dinucleotide-related molecule is selected from the group consisting of oxidized nicotinamide adenine dinucleotide, reduced nicotinamide adenine dinucleotide, oxidized nicotinamide adenine dinucleotide phosphate, reduced nicotinamide adenine dinucleotide phosphate, and a mimetic thereof.

37. A method for constructing a pharmacaphore model, comprising constructing a model that contains one or more selected conformation-dependent properties of one or more pharmacoclusters.

38. A method for identifying a binding compound for one or more members of a polypeptide pharmacofamily, comprising identifying a compound having a selected conformation-dependent property of a pharmacocluster.

39. A pharmacocluster selected from the group consisting of pharmacocluster 1, pharmacocluster 2, pharmacocluster 3, pharmacocluster 4, pharmacocluster 5, pharmacocluster 6, pharmacocluster 7, and pharmacocluster 8.

40. A polypeptide pharmacofamily, comprising polypeptides that bind to substantially the same bound conformation of a nicotinamide adenine dinucleotide-related molecule selected from pharmacofamily 1, pharmacofamily 2, pharmacofamily 3, pharmacofamily 4, pharmacofamily 5, pharmacofamily 6, pharmacofamily 7, and pharmacofamily 8.

41. A polypeptide pharmacofamily, comprising polypeptides that bind to a nicotinamide adenine dinucleotide-related molecule having a bound conformation selected from pharmacocluster 1, pharmacocluster 2, pharmacocluster 3, pharmacocluster 4, pharmacocluster 5, pharmacocluster 6, pharmacocluster 7, and pharmacocluster 8.

42. A ligand conformer model, comprising a ligand conformer model, selected from the group consisting of conformer model 1 having coordinates listed in Table 3C, conformer model 2 having coordinates listed in Table 4C, conformer model 3 having coordinates listed in Table 5C, conformer model 4 having coordinates listed in Table 6C, conformer model 5 having coordinates listed in Table 7C, conformer model 6 having coordinates listed in Table 8C, conformer model 7 having coordinates listed in Table 9C, and conformer model 8 having coordinates listed in Table 10C.

43. A moiety, comprising coordinates, selected from the group consisting of coordinates listed in Table 3C, coordinates listed in Table 4C, coordinates listed in Table 5C, coordinates listed in Table 6C, coordinates listed in Table 7C, coordinates listed in Table 8C, coordinates listed in Table 9C, and coordinates listed in Table 10C.

44. A pharmacophore model, comprising a pharmacophore model selected from the group consisting of pharmacophore model 1 having coordinates listed in Tables 3B and 3C, pharmacophore model 2 having coordinates listed in Tables 4B and 4C, pharmacophore model 3 having coordinates listed in Tables 5B and SC, pharmacophore model 4 having coordinates listed in Tables 6B and 6C, pharmacophore model 5 having coordinates listed in Tables 7B and 7C, pharmacophore model 6 having coordinates listed in Tables 8B and 8C, pharmacophore model 7 having coordinates listed in Tables 9B and 9C, and pharmacophore model 8 having coordinates listed in Tables 10B and 10C.
